(19) United States
(12) Patent Application Publication
(10) Pub. No.: US 2016/0178520 A1
(43) Pub. Date: Jun. 23, 2016

(54) Multiplexed Single Molecule Analyzer

(71) Applicant: Singulex, Inc., Alameda, CA (US)

(72) Inventors: Richard Livingston, Webster Groves, MO (US); Michele Gilbert, Albany, CA (US); Jacqueline Felberg, Alameda, CA (US); Jeffrey Bishop, Dublin, CA (US)

(21) Appl. No.: 14/973,306

(22) Filed: Dec. 17, 2015

Related U.S. Application Data
(60) Provisional application No. 62/093,315, filed on Dec. 17, 2014.

(51) Int. Cl. G01N 21/64; G01N 33/536; G01N 33/68; G01N 33/53

(52) U.S. Cl. CPC G01N 21/6428; G01N 33/5302; G01N 33/536; G01N 33/6845; G01N 2021/6441; G01N 2201/0612

(57) ABSTRACT

Analyzers and analyzer systems that include an analyzer for determining multiple label species, methods of using the analyzer and analyzer systems to analyze samples, are disclosed herein. The analyzer includes one or more sources of electromagnetic radiation to provide electromagnetic radiation at wavelengths within the excitation bands of one or more fluorophore species to an interrogation space that is translated through the sample to detect the presence or absence of molecules of different target analytes. The analyzer may also include one or more detectors configured to detect electromagnetic radiation emitted from the one or more fluorophore species. The analyzer for determining multiple target molecule species provided herein is useful for diagnostics because the concentration of multiple species of target molecules may be determined in a single sample and with a single system.

### CROSS-REFERENCE TO RELATED APPLICATION

This application claims the benefit of U.S. Provisional Patent Application No. 62/093,315, filed Dec. 17, 2014, which is incorporate herein by reference in its entirety.

### BACKGROUND

Unless otherwise indicated herein, the materials described in this section are not prior art to the claims in this application and are not admitted to be prior art by inclusion in this section.

All publications and patent applications mentioned in this specification are herein incorporated by reference to the same extent as if each individual publication or patent application was specifically and individually indicated to be incorporated by reference. Advances in biomedical research, medical diagnosis, prognosis, monitoring and treatment selection, bioterrorism detection, and other fields involving the analysis of multiple samples of low volume and concentration of analytes have led to development of sample analysis systems capable of sensitively detecting particles in a sample at ever-decreasing concentrations. U.S. Pat. Nos. 4,793,705, 5,209,834, and 8,264,684, which are incorporated herein by reference, describe previous systems that achieved extremely sensitive detection. The disclosure provides further development in this field.

### SUMMARY

Embodiments of the present disclosure provide an analyzer including: (a) an electromagnetic radiation source for providing electromagnetic radiation to a sample container for a sample, wherein the electromagnetic radiation source provides electromagnetic radiation at wavelengths within at least an excitation band of a first label corresponding to a first target molecule and within an excitation band of a second label corresponding to a second target molecule; (b) a system for directing the electromagnetic radiation from the electromagnetic radiation source to an interrogation space in the sample; (c) a translating system for translating the interrogation space through at least a portion of the sample, thereby forming a moveable interrogation space; and (d) a detector system comprising a first detector for detecting electromagnetic radiation emitted in the interrogation space by the first label if the first label is present in the interrogation space and a second detector for detecting electromagnetic radiation emitted in the interrogation space by the second label if the second label is present in the interrogation space.

Some embodiments of the present disclosure provide an analyzer including: (a) an electromagnetic radiation source for providing electromagnetic radiation to a sample container for a sample, wherein the electromagnetic radiation source provides electromagnetic radiation at wavelengths within at least an excitation band of a first fluorescent moiety and within an excitation band of a second fluorescent moiety; (b) a system for directing the electromagnetic radiation from the electromagnetic radiation source to an interrogation space in the sample; (c) a translating system for translating the interrogation space through at least a portion of the sample, thereby forming a moveable interrogation space; (d) a detector system comprising a first detector for detecting electromagnetic radiation emitted in the interrogation space by the first fluorescent moiety, a second detector for detecting electromagnetic radiation emitted in the interrogation space by the second fluorescent moiety during a plurality of bin times; and (e) a processor configured to: (i) determine a first threshold photon value corresponding to a background signal in the interrogation space at an emission wavelength of the first label, (ii) determine a second threshold photon value corresponding to a background signal in the interrogation space at an emission wavelength of the second label, (iii) determine a first analyte by determining the first fluorescent moiety corresponding to the first analyte in the interrogation space by identifying each bin of a first plurality of bins having a photon value for the first moiety greater than the first threshold value; (iv) determine a second analyte by determining the second fluorescent moiety corresponding to the second analyte in the interrogation space by identifying each bin of a second plurality of bins having a photon value for the second moiety greater than the second threshold value; and (v) determine a third analyte by determining a combination of the first fluorescent moiety and the second fluorescent moiety corresponding to the third analyte in the interrogation space by identifying each bin of a third plurality of bins having a photon value for each of the first moiety and the second moiety greater than the first and the second threshold values.

Further embodiments of the present disclosure provide an analyzer including: (a) a first electromagnetic radiation source for providing electromagnetic radiation at an excitation wavelength to a sample container for a sample and a second electromagnetic radiation source for providing electromagnetic radiation at an excitation wavelength to the sample container; (b) a system for directing the electromagnetic radiation from the first electromagnetic radiation source and the second electromagnetic radiation source to at least one interrogation space in the sample; (c) a translating system for translating the interrogation space through at least a portion of the sample, thereby forming a moveable interrogation space; and (d) a detection system for detecting electromagnetic radiation, wherein the detection system is configured to detect electromagnetic radiation from the interrogation space emitted by: (i) a first label corresponding to the first target molecule if the first label is present in the at least one interrogation space, wherein the first label has an excitation wavelength overlapping the excitation wavelength of the first electromagnetic radiation source; and (ii) the second label corresponding to a single molecule of the second target molecule if the second label is present in the at least one interrogation space, wherein the second label has an excitation wavelength overlapping the excitation wavelength of the second electromagnetic radiation source.

Embodiments of the present disclosure also provide a method for determining multiple target molecules including: (a) directing electromagnetic radiation from an electromagnetic radiation source to an interrogation space in a sample at a first wavelength within at least an excitation band of a first label corresponding to a first target molecule and a second wavelength within at least an excitation band of a second label corresponding to a second target molecule; (b) detecting the first label in the interrogation space located at a first position in the sample; (c) detecting the second label in the interrogation space at the first position; (d) translating the interrogation space through the sample to a subsequent position in the sample; (e) detecting the first label in the interrogation space located at the subsequent position in the sample; (f) detecting second label in the interrogation space located at the subsequent position in the sample; and (g) repeating steps (d), (e) and (f) as required to detect the first label and the second label in more than one position of the sample, thereby determining the first target molecule and the second target molecule.

Still further embodiments of the present disclosure provide a method for determining a target molecule including: (a) directing electromagnetic radiation from a first electromagnetic radiation source at a first wavelength to a first interrogation space in a sample and directing electromagnetic radiation from a second electromagnetic radiation source at a second wavelength to a second interrogation space in the sample, wherein the first and second interrogation spaces are within a focus of a single objective of a detector; (b) detecting with a first detector the first label corresponding to a first target molecule in the first interrogation space at the first position in the sample; wherein the first label has an excitation wavelength within the first wavelength, wherein electromagnetic radiation emitted in the first interrogation space is directed to the first detector; (c) detecting with a second detector a second label corresponding to second target molecule in the second interrogation space at the first position in the sample, wherein the second label has an excitation wavelength within the second wavelength, wherein electromagnetic radiation emitted in the second interrogation space is directed to the second detector; (d) translating the interrogation space through the sample to a subsequent position in the sample; (e) detecting with the first detector the first label in the first interrogation space at the subsequent position in the sample; (f) detecting with the second detector the second label in the second interrogation space at the subsequence position in the sample; and (g) repeating steps (d), (e) and (f) as required to determine the first target molecule and the second target molecule.

### BRIEF DESCRIPTION OF THE DRAWINGS

The novel features of the disclosure are set forth with particularity in the appended claims. A better understanding of the features and advantages of the disclosure will be obtained by reference to the following detailed description that sets forth illustrative embodiments, in which the principles of the disclosure are utilized, and the accompanying drawings of which:

FIG. 1A illustrates an embodiment of a scanning single molecule analyzer as viewed from the top.

FIG. 1B illustrates the scanning single molecule analyzer of FIG. 1A as viewed from the side.

FIG. 2 depicts a graph showing the diffusion time for a 155 KDa molecular weight molecule as a function of the diffusion radius of the molecule.

FIG. 3 shows example detection event data generated using an embodiment of a scanning single molecule analyzer.

FIG. 4 shows example standard curves generated with a scanning single molecule analyzer by detecting a sample over a range of known concentrations.

FIG. 5A shows an example schematic diagram of a multiplexed single molecule analyzer system.

FIG. 5B shows an example system having multiple detectors.

FIG. 6A shows an example schematic diagram of a multiplexed single molecule analyzer system.

FIG. 6B shows an example system having multiple electromagnetic radiation sources.

FIG. 7A shows an example schematic diagram of a multiplexed single molecule analyzer system.

FIG. 7B shows an example system having multiple electromagnetic radiation sources and multiple detectors.

FIG. 8 illustrates separated detection spots within a single sample well and separated detection spots.

FIG. 9A is a plot of raw detection event counts of green fluorophores excited with a 635 nm laser.

FIG. 9B is a plot of raw detection event counts of green fluorophores excited with a 405 nm laser.

FIG. 9C is a plot of raw detection event counts of red fluorophores excited with a 520 nm laser.

FIG. 9D is a plot of raw detection event counts of red fluorophores excited with a 405 nm laser.

FIG. 9E is a plot of raw detection event counts of blue fluorophores excited with a 635 nm laser.

FIG. 10A is a plot illustrating the impact of 635 nm laser irradiation on photobleaching of fluorophore BV421.

FIG. 10B is a plot illustrating the impact of 635 nm laser irradiation on photobleaching of fluorophore FP532A.

FIG. 10C is a plot illustrating the impact of 635 nm laser irradiation on photobleaching of fluorophore ATTO532.

FIG. 10D is a plot illustrating the impact of 405 nm laser irradiation on photobleaching of fluorophore Alexa 647.

FIG. 10E is a plot illustrating the impact of 520 nm laser irradiation on photobleaching of fluorophore Alexa 647.

FIG. 11A is a plot of the excitation and emission spectra for fluorophores ALEXA FLUOR® 405 and Cascade Blue, excitation filter, and emission filter.

FIG. 11B is a plot of the excitation and emission spectra for fluorophore ALEXA FLUOR® 532, excitation filter, and emission filter.

FIG. 11C is a plot of the excitation and emission spectra for fluorophore ALEXA FLUOR® 647, excitation filter, and emission filter.

FIG. 11D is a plot of the excitation and emission spectra for fluorophore ALEXA FLUOR® 790, excitation filter, and emission filter.

FIGS. 12A and 12B show a flow chart of an example method

FIGS. 13A and 13B show a flow chart of an example method.

FIG. 14A is a plot of a reference limit calculation for healthy human plasma samples tested for example analytes.

FIG. 14B is a plot of a reference limit calculation for healthy human plasma samples tested for example analytes.

FIG. 14C is a plot of a reference limit calculation for healthy human plasma samples tested for example analytes.

### DETAILED DESCRIPTION

While embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Overview

The disclosure provides systems and methods for highly sensitive detection and quantitation of one or more target molecules, such as markers for biological states. Such systems, which may include instruments, kits, and compositions, may be referred to as “single molecule detectors,” “single particle detectors,” “single molecule analyzers,” “single particle analyzers,” “single molecule readers,” or “single particle readers.” Compositions and methods for diagnosis, prognosis, and/or determination of treatment based on such highly sensitive detection and quantization are also described.

In one aspect, the disclosure provides systems and methods that can perform a “singleplex” assay of a sample to detect and analyze a single type of target molecule in the sample. In other aspects, the disclosure provides systems and methods that can perform a “multiplex” assay of a sample to detect and analyze multiple (e.g., two, three or more) different types of target molecules in the sample. Using the multiplexed systems and methods described herein may provide for more rapid detection and analysis of multiple target molecules, using reduced sample volume, and reduced reagent volume than may be required to perform a similar analysis of those target molecules via singleplex assays. Among other scenarios, this may be beneficial when conducting analyses relating to disease states and biochemical pathways represented by multiple target molecules (e.g., biomarkers).

In some multiplex assay examples, a multiplexed analyzer system includes one or more electromagnetic radiation sources that provide radiation to a sample located within a sample container. The sample can include multiple, different target molecules labeled with one or more types of labels. The multiplexed analyzer system also includes a system for directing the electromagnetic radiation from the electromagnetic radiation source(s) to one or more interrogation space(s) in the sample. The multiplexed analyzer system further includes a translating system for translating the interrogation space(s) through at least a portion of the sample, thereby forming moveable interrogation space(s). As the interrogation space(s) translates over a label, at least one of the electromagnetic radiation source(s) may cause the label to emit a detectable amount of energy. The multiplexed analyzer system includes one or more detectors operably coupled to the interrogation space(s) such that the detector(s) detect energy emitted from the labels in the interrogation space(s) if the corresponding target molecules are present is the sample or in a sample that was processed to provide a processing sample that contains one label corresponding to each target molecule in a sample. For instance, a processing sample being analyzed contains a single labeled antibody for each molecule of a protein that binds the antibody in the original sample. The labeled antibody may or may not be bound to the protein in the processing sample. In some implementations, the multiplexed analyzer system can further include a processor that can analyze the detected energy to detect the presence and/or determine a concentration of the each type of target molecule in the sample.

To detect and analyze multiple, different types of target molecules in a sample, the multiplexed analyzer system can distinguish one type of target molecule from the others. This can be achieved, in part, by labeling the different target molecules with different labels, which have excitation wavelength bands and/or emission wavelength bands that differ from one another. In some implementations, the different labels have excitation wavelength bands and/or emission wavelength bands with relatively little overlap or no overlap. In other implementations, there may be some overlap among the excitation wavelength bands and/or the emission wavelength bands of the labels.

In the description below, example singleplex aspects are described first and then example multiplex aspects are described.

Example Scanning Single Molecule Analyzer

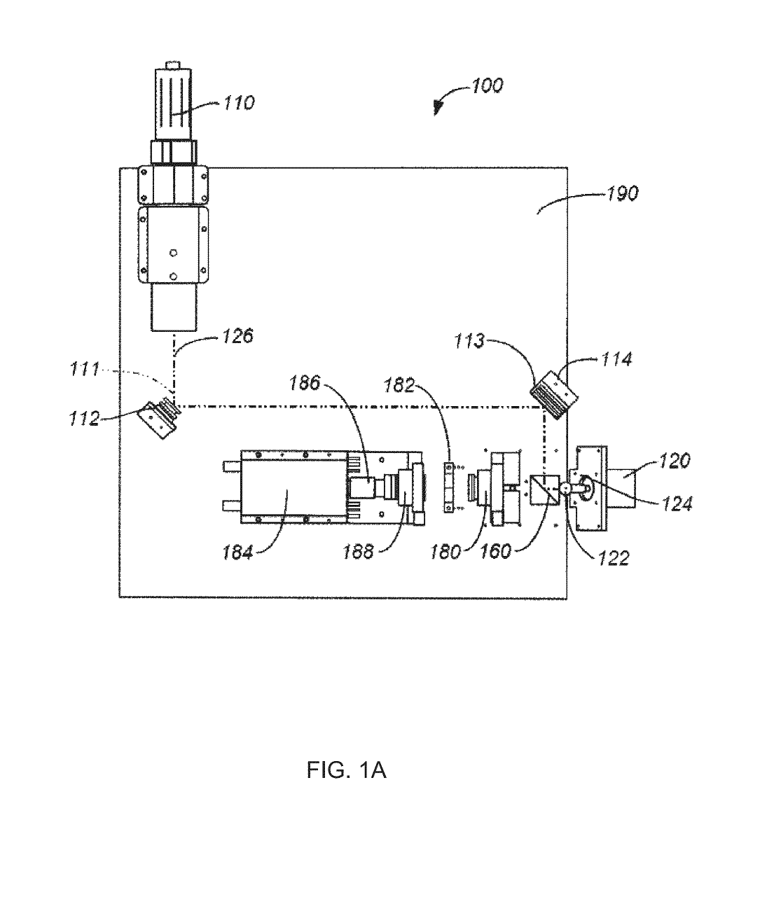

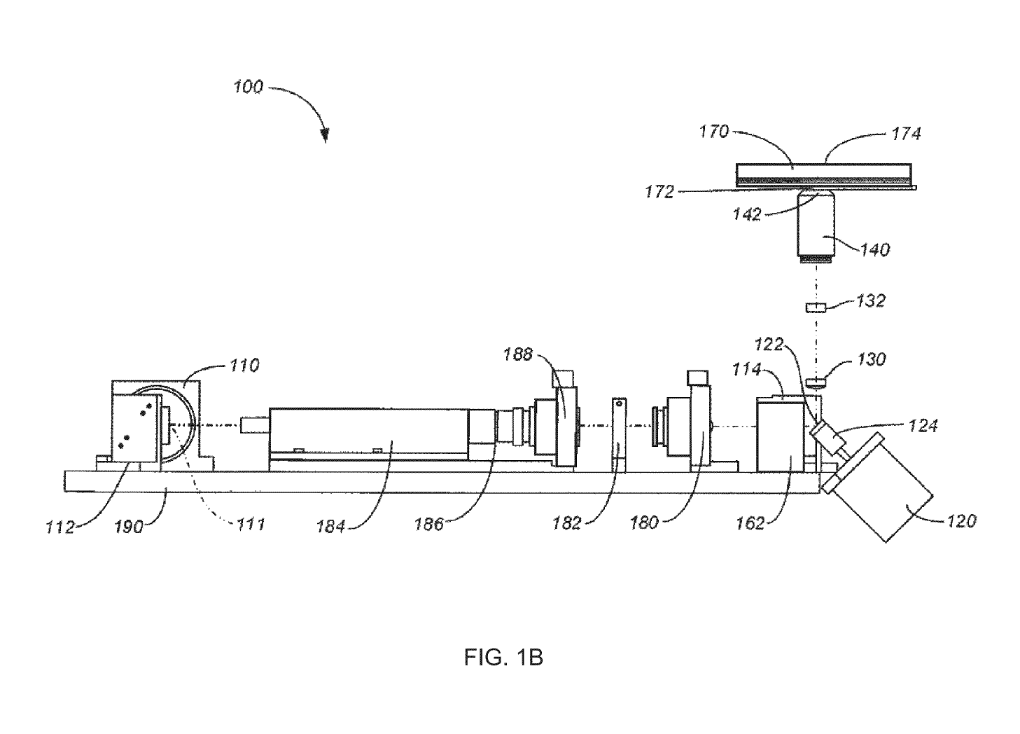

As shown in FIGS. 1A and 1B, described herein is one embodiment of a scanning analyzer system 100. The analyzer system 100 includes electromagnetic radiation source 110, a first alignment mirror 112, a second alignment mirror 114, a dichroic mirror 160, a rotating scan mirror 122 mounted to the shaft 124 of a scan motor 120. As shown in FIG. 1B, the rotating scan mirror 122 deflects the electromagnetic radiation source through a first scan lens 130, through a second scan lens 132, and through a microscope objective lens 140, to a sample plate 170. The fluorescence associated with the single molecules contained on or in the sample plate 170 is detected using a tube lens 180, an aperture 182, a detector filter 188, a detector lens 186, and a detector 184. The signal is then processed by a processor (not shown) operatively coupled to the detector 184. In some embodiments, the entire scanning analyzer system 100 is mounted to a baseboard 190.

In operation, the electromagnetic radiation source 110 is aligned so that its output 126, e.g., a beam, is reflected off the front surface 111 of a first alignment mirror 112 to the front surface 113 of a second alignment mirror 114 to the dichroic mirror 160 mounted to a dichroic mirror mount 162. The dichroic mirror 160 then reflects the electromagnetic radiation 126 to the front surface of a scan mirror 122 located at the tip of the shaft 124 of the scan motor 120. The electromagnetic radiation 126 then passes through a first scan lens 130 and a second scan lens 132 to the microscope objective lens 140. The objective lens 140 focuses the beam 126 through the base 174 of the sample plate 170 and directs the beam 126 to an interrogation space located on the opposite side of the sample plate 170 from which the beam 126 entered. Passing the electromagnetic radiation beam 126 through a first scan lens 130 and a second scan lens 132 ensures all light to the objective lens 140 is coupled efficiently. The beam 126 excites the label attached to the single molecule of interest contained on or in the sample plate 170. The label emits radiation that is collected by the objective 140. The electromagnetic radiation is then passed back through the scan lenses 130, 132 which then ensure coupling efficiency of the radiation from the objective 140. The detected radiation is reflected off of the front surface of the scan mirror 122 to the dichroic mirror 160. Because the fluorescent light detected is different than the color of the electromagnetic radiation source 110, the fluorescent light passing the dichroic mirror 160 passes through a tube lens 180, an aperture 182, a detector filter 188 and detector lens 186 to a detector 184. The detector filter 188 minimizes aberrant noise signals due to light scatter or ambient light while maximizing the signal emitted by the excited fluorescent moiety bound to the particle. A processor processes the light signal from the particle according to the methods described herein.

In one embodiment, the microscope objective 140 has a numerical aperture. As used herein, “high numerical aperture lens” includes a lens with a numerical aperture of equal to or greater than 0.6. The numerical aperture is a measure of the number of highly diffracted image-forming light rays captured by the objective. A higher numerical aperture allows increasingly oblique rays to enter the objective lens and thereby produce a more highly resolved image. The brightness of an image also increases with higher numerical aperture. High numerical aperture lenses are commercially available from a variety of vendors, and any one lens having a numerical aperture of equal to or greater than approximately 0.6 can be used in the analyzer system. In some examples, the lens may have a numerical aperture falling within the range of 0.6 to about 1.3, in particular, 0.6 to about 1.0, 0.7 to about 1.2, 0.7 to about 1.0, 0.7 to about 0.9, 0.8 to about 1.3, 0.8 to about 1.2, or 0.8 to about 1.0. In some embodiments, the lens has a numerical aperture of at least about 0.6, for example, at least about 0.7, at least about 0.8, at least about 0.9, or at least about 1.0. In some embodiments, the aperture of the microscope objective lens 140 is approximately 1.25.

The high numerical aperture (NA) microscope objective, used when performing single molecule detection through the walls or the base of the sample plate 170, has short working distances. The working distance is the distance from the front of the lens to the object in focus. The objective in some embodiments can be within 350 microns of the object. In some embodiments, where a microscope objective lens 140 with NA of 0.8 is used, an Olympus 40×/0.8 NA water immersion objective (Olympus America, Inc., USA) can be used. This objective has a 3.3 mm working distance. In some embodiments, an Olympus 60×/0.9 NA water immersion objective with a 2 mm working distance can be used. Because the later lens is a water immersion lens, the space 142 between the objective and the sample can be filled with water. This can be accomplished using a water bubbler (not shown) or some other suitable plumbing for depositing water between the objective and the base of the sample plate.

The electromagnetic radiation source is set so that the wavelength of the laser is sufficient to excite the fluorescent label attached to the particle. In some embodiments, the electromagnetic radiation source 110 is a laser that emits light in the visible spectrum. In some embodiments, the laser is a continuous wave laser with a wavelength of 639 nm, 532 nm, 488 nm, 422 nm, or 405 nm. Any continuous wave laser with a wavelength suitable for exciting a fluorescent moiety as used in the methods and compositions of the disclosure can be used without departing from the scope of the disclosure.

As the interrogation space in the single molecule analyzer system 100 passes over the labeled single molecule, the beam 126 of the electromagnetic radiation source directed into the interrogation space causes the label to enter an excited state. When the particle relaxes from its excited state, a detectable burst of light is emitted. In the length of time it takes for the interrogation space to pass over the particle, the excitation-emission cycle is repeated many times by each particle. This allows the analyzer system 100 to detect tens to thousands of photons for each particle as the interrogation space passes over the particle. Photons emitted by the fluorescent particles are registered by the detector 184 with a time delay indicative of the time for the interrogation space to pass over the labeled particle. The photon intensity is recorded by the detector 184 and the sampling time is divided into bins, wherein the bins are uniform, arbitrary time segments with freely selectable time channel widths. The number of signals contained in each bin is evaluated. One or more of several statistical analytical methods are used to determine when a particle is present. As will be discussed further below, these methods include determining the baseline noise of the analyzer system and determining signal strength for the fluorescent label at a statistical level above baseline noise to mitigate false positive signals from the detector.

Electromagnetic Radiation Source

Some embodiments of the analyzer system use a chemiluminescent label. These embodiments may not require an electromagnetic radiation source for particle detection. In other embodiments, the extrinsic label or intrinsic characteristic of the particle is light-interacting, such as a fluorescent label or light-scattering label. In such an embodiment, a source of EM radiation is used to illuminate the label and/or the particle. EM radiation sources for excitation of fluorescent labels are preferred.

In some embodiments, the analyzer system consists of an electromagnetic radiation source 110. Any number of radiation sources can be used in a scanning analyzer system 100 without departing from the scope of the disclosure. For example, the electromagnetic radiation source 110 can be a continuous wave laser producing wavelengths of between 200 nm and 1000 nm. Continuous wave lasers provide continuous illumination without accessory electronic or mechanical devices, such as shutters, to interrupt their illumination. Such electromagnetic radiation sources have the advantage of being small, durable and relatively inexpensive. In addition, they generally have the capacity to generate larger fluorescent signals than other light sources. Specific examples of suitable continuous wave electromagnetic radiation sources include, but are not limited to: lasers of the argon, krypton, helium-neon, helium-cadmium types, as well as, diode lasers (red to infrared regions), each with the possibility of frequency doubling. In an embodiment where a continuous wave laser is used, an electromagnetic radiation source of less than 3 mW, for example 2 mW and 1 mW, may have sufficient energy to excite a fluorescent label depending on the label selected. A beam of such energy output can be between 2 to 5 μm in diameter. When exposed at 3 mW, a labeled particle can be exposed to the laser beam for about 1 msec, equal to or less than about 500 μsec, equal to or less than about 100 μsec, equal to or less than about 50 μsec, or equal to or less than about 10 μsec.

Light-emitting diodes (LEDs) are another low-cost, highly reliable illumination source. Advances in ultra-bright LEDs and dyes with high absorption cross-section and quantum yield have made LEDs applicable for single molecule detection. Such LED light can be used for particle detection alone or in combination with other light sources such as mercury arc lamps, elemental arc lamps, halogen lamps, arc discharges, plasma discharges, and any combination of these.

The electromagnetic radiation source can also comprise a pulse wave laser. In such an embodiment, the pulse size, size, focus spot, and total energy emitted by the laser may be sufficient to excite the fluorescent label. In some embodiments, a laser pulse of less than 1 nanosecond can be used. A pulse of this duration can be preferable in some pulsed laser applications. In other embodiments, a laser pulse of 1, 2, 3, 4 or 5 nanoseconds can be used. In still other embodiments, a pulse of between 2 to 5 nanoseconds can be used. In other embodiments, a pulse of longer duration can be used.

The optimal laser intensity depends on the photo bleaching characteristics of the single dyes and the length of time required to traverse the interrogation space (including the speed of the particle, the distance between interrogation spaces if more than one is used and the size of the interrogation space(s)). To obtain a maximal signal, the sample can be illuminated at the highest intensity that will not photo bleach a high percentage of the dyes. The preferred intensity is such that no more that 5% of the dyes are bleached by the time the particle has traversed the interrogation space.

The power of the laser is set depending on the type of dye molecules and the length of time the dye molecules are stimulated. The power can also depend on the speed that the interrogation space passes through the sample. Laser power is defined as the rate at which energy is delivered by the beam and is measured in units of Joules/second, or Watts. To provide a constant amount of energy to the interrogation space as the particle passes through, the less time the laser can illuminate the particle as the power output of the laser is increased. In some embodiments, the combination of laser power and illumination time is such that the total energy received by the interrogation space during the time of illumination is more than about 0.1, 0.5, 1, 2, 3, 4, 5, 6, 7, 8, 9, 10, 12, 15, 20, 25, 30, 35, 40, 45, 50, 60, 70, 80, 90, 100, or 110 microJoule. In some embodiments, the combination of laser power and illumination time is such that the total energy received by the interrogation space during the time of illumination is less than about 0.5, 1, 2, 3, 4, 5, 6, 7, 8, 9, 10, 12, 15, 20, 25, 30, 35, 40, 45, 50, 60, 70, 80, 90, 100, or 110 microJoule. In some embodiments, the combination of laser power and illumination time is such that the total energy received by the interrogation space during the time of illumination is between about 0.1 and 100 microJoule, for example, between about 1 and 100 microJoule, between about 1 and 50 microJoule, between about 2 and 50 microJoule, between about 3 and 60 microJoule, between about 3 and 50 microJoule, between about 3 and 40 microJoule, or between about 3 and 30 microJoule. In some embodiments, the combination of laser power and illumination time is such that the total energy received by the interrogation space during the time of illumination is about 1 microJoule, about 3 microJoule, about 5 microJoule, about 10 microJoule, about 15 microJoule, about 20 microJoule, about 30 microJoule, about 40 microJoule, about 50 microJoule, about 60 microJoule, about 70 microJoule, about 80 microJoule, about 90 microJoule, or about 100 microJoule.

In some embodiments, the laser power output is set to at least about 1 mW, 2 mW, 3 mW, 4 mW, 5 mW, 6 mW, 7 mW, 8 mW, 9 mW, 10 mW, 13 mW, 15 mW, 20 mW, 25 mW, 30 mW, 40 mW, 50 mW, 60 mW, 70 mW, 80 mW, 90 mW, 100 mW, or more than 100 mW. In some embodiments, the laser power output is set to at least about 1 mW, at least about 3 mW, at least about 5 mW, at least about 10 mW, at least about 15 mW, at least about 20 mW, at least about 30 mW, at least about 40 mW, at least about 50 mW, at least about 60 mW, or at least about 90 mW.

The time that the laser illuminates the interrogation space can be set to no less than about 1, 2, 3, 4, 5, 10, 15, 20, 30, 40, 50, 60, 70, 80, 90, 100, 150, 200, 250, 300, 350, 400, 450, 500, 600, 700, 800, 900, 1000, 1500 or 2000 microseconds. The time that the laser illuminates the interrogation space can be set to no more than about 2, 3, 4, 5, 10, 15, 20, 30, 40, 50, 60, 70, 80, 90, 100, 150, 200, 250, 300, 350, 400, 450, 500, 600, 700, 800, 900, 1000, 1500, or 2000 microseconds. The time that the laser illuminates the interrogation space can be set between about 1 and 1000 microseconds. For example, the time that the laser illuminates the interrogation space can be set between about 5 and 500 microseconds, between about 5 and 100 microseconds, between about 10 and 100 microseconds, between about 10 and 50 microseconds, between about 10 and 20 microseconds, between about 5 and 50 microseconds, or between about 1 and 100 microseconds. In some embodiments, the time that the laser illuminates the interrogation space is about 1 microsecond, about 5 microseconds, about 10 microseconds, about 25 microseconds, about 50 microseconds, about 100 microseconds, about 250 microseconds, about 500 microseconds, or about 1000 microseconds.

In some embodiments, the laser illuminates the interrogation space for 1 millisecond, 250 microseconds, 100 microseconds, 50 microseconds, 25 microseconds or 10 microseconds with a laser that provides a power output of 1 mW, 2 mW, 3 mW, 4 mW, 5 mW, or more than 5 mW. In some embodiments, a label is illuminated with a laser that provides a power output of 3 mW and illuminates the label for about 1000 microseconds. In other embodiments, a label is illuminated for less than 1000 milliseconds with a laser providing a power output of not more than about 20 mW. In other embodiments, the label is illuminated with a laser power output of 20 mW for less than or equal to about 250 microseconds. In some embodiments, the label is illuminated with a laser power output of about 5 mW for less than or equal to about 1000 microseconds.

Optical Scanning System

The scanning analyzer system described herein is, in some embodiments, different than traditional single molecule analyzers previously described elsewhere. In flow cytometry and other methods of fluorescence spectroscopy, a sample flows through an interrogation space. In contrast, the interrogation space in one embodiment of the analyzer provided herein is moved relative to the sample. This can be done by fixing the sample container relative to the instrument and moving the electromagnetic radiation beam. Alternatively, the electromagnetic radiation beam can be fixed and the sample plate moved relative to the beam. In some embodiments, a combination of both can be used. In an embodiment wherein the sample plate is translated to create the moveable interrogation space, the limiting factor is the ability to move the plate smoothly enough so that the sample located on the sample plate is not jarred and the interrogation space is in the desired location.

In one embodiment, the electromagnetic radiation source 110 is focused onto a sample plate 170 of the analyzer system 100. The beam 126 from the continuous wave electromagnetic radiation source 110 is optically focused through the base of the sample plate to a specified depth plane within the sample contained on or in the sample plate 170. Optical scanning of the sample can be accomplished using mirrors or lenses. In some embodiments, a mirror 122 is mounted on the end of a scan motor shaft 124 of the scan motor 120 but is tilted at a slight angle relative to the shaft 124. In some embodiments, as the mirror 122 turns, it can deflect the electromagnetic radiation beam 126 thereby creating a small circle. By placing the mirror 122 between the objective 140 and the dichroic mirror 160, the spot at the focus of the objective can move around the sample. In some embodiments, the sample is scanned in a circular pattern. In such an embodiment, a scan circle with a diameter of between about 500 μm and about 750 μm can be formed. In some embodiments, a scan circle with a diameter of between about 550 μm and 700 μm can be formed. In some embodiments, a scan circle with a diameter of between about 600 μm and 650 μm can be formed. In some embodiments a scan circle with a diameter of about 630 μm can be formed. In some embodiments, when a scan circle with a diameter of 630 μm is used, the scan circle can be traversed at about 8 revolutions per second (or about 500 RPM), equivalent to pumping the sample through a flow source at a rate of about 5 μl/min.

In some embodiments, the scan speed of the interrogation space is more than 100 RPM, is more than 300 RPM, is more than 500 RPM, is more than 700 RPM, or is more than 900 RPM. In some embodiments, the scan speed of the interrogation space is less than 1000 RPM, is less than 800 RPM, is less than 600 RPM, is less than 400 RPM, of is less than 200 RPM. In some embodiments, the scan speed of the interrogation space is between about 100 RPM and about 1000 RPM, between about 200 RPM and about 900 RPM, between about 300 RPM and about 800 RPM, between about 400 RPM and about 700 RPM, between about 450 RPM and about 600 RPM, or between about 450 RPM and about 550 RPM. With the development of improved electronics and optics, scanning in the z-axis may be required in addition to scanning in a two-dimensional pattern to avoid duplicate scanning of the same molecule. In some of the embodiments previously mentioned, the optical scanning pattern allows the scanning of a substantially different volume each time a portion of the sample is scanned.

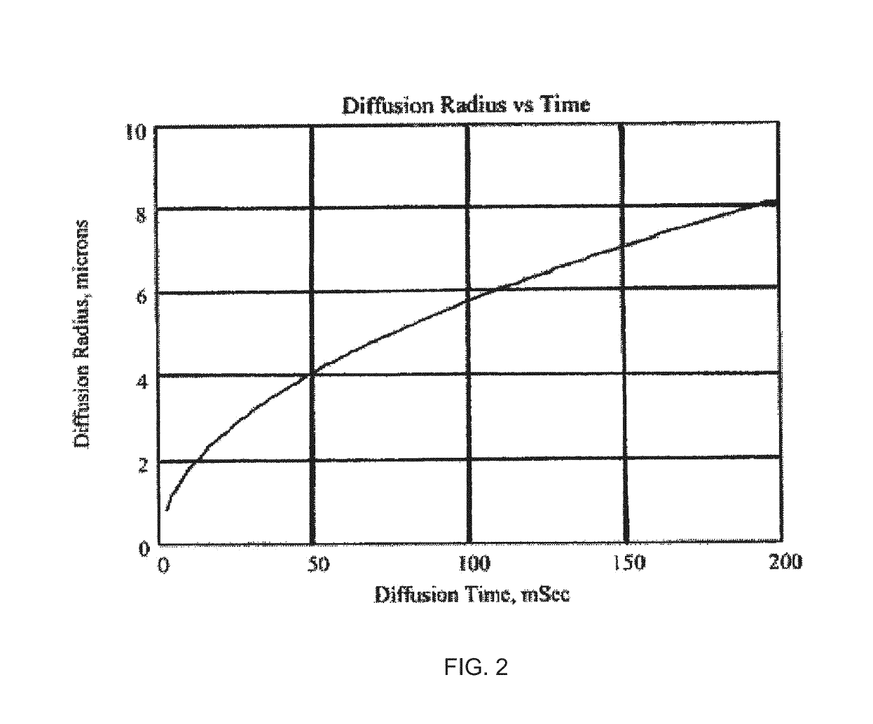

The sample is scanned by an electromagnetic radiation source that interrogates a portion of the sample. A single molecule of interest may or may not be present in the interrogation space. In some embodiments, a portion of the sample is scanned a first time and then subsequently scanned a second time. In some embodiments the same portion of sample is scanned multiple times. In some embodiments, the sample is scanned such that the detection spot returns to a portion of sample a second time after sufficient time has passed so that the molecules detected in the first pass have drifted or diffused out of the portion, and other molecules have drifted or diffused into the portion. When the same portion of sample is scanned at least one or more times, the scanning speed can be slow enough to allow molecules to diffuse into, and out of, the space being interrogated. In some embodiments, the interrogation space is translated through a same portion of sample a first time and a second time at a sufficiently slow speed as to allow a molecule of interest that is detected the first time the interrogation space is translated through the portion of sample to substantially diffuse out of the portion of sample after the first time the portion of sample is interrogated by the interrogation space, and to further allow a subsequent molecule of interest, if present, to substantially diffuse into the portion of sample the second time the portion of sample is interrogated by the interrogation space. FIG. 2 shows a graph of the diffusion time versus corresponding diffusion radius for molecules with a 155 KDa molecular weight. As used herein, “diffusion radius” refers to the standard deviation of the distance from the starting point that the molecule will most likely diffuse in the time indicated on the X-axis.

In some embodiments an alternative scan pattern is used. In some embodiments, the scan pattern can approximate an arc. In some embodiments, the scan pattern comprises at least one 90 degree angle. In some embodiments, the scan pattern comprises at least one angle less than 90 degrees. In some embodiments, the scan pattern comprises at least one angle that is more than 90 degrees. In some embodiments, the scan pattern is substantially sinusoidal. In some embodiments, the optical scanning can be done with one mirror as previously described. In an alternative embodiment, the optical scanning can be done with at least two mirrors. Multiple mirrors allow scanning in a straight line, as well as allowing the system to scan back and forth, so that a serpentine pattern is created. Alternatively, a multiple mirror optical scanning configuration allows for scanning in a raster pattern.

In an alternative embodiment, optical scanning can be done using an optical wedge. A wedge scanner provides a circular scan pattern and shortens the optical path because scan lenses are not required. An optical wedge approximates a prism with a very small angle. The optical wedge can be mounted to the shaft of the electromagnetic radiation source. The optical wedge rotates to create an optical scan pattern. In an alternative embodiment, the scan mirror can be mounted using an electro-mechanical mount. In such an embodiment, the electro-mechanical mount would have two voice coils. One voice coil would cause displacement of the mirror in a vertical direction. The other voice coil would cause displacement of the mirror in a horizontal direction. Using this embodiment, any scan pattern desired can be created.

The scanning particle analyzer can scan the sample located in the sample plate in a two-dimensional orientation, e.g., following the x-y plane of the sample. In some embodiments, the sample can be scanned in a three-dimensional orientation consisting of scanning in an x-y plane and z direction. In some embodiments, the sample can be scanned along the x-y and z directions simultaneously. For example, the sample can be scanned in a helical pattern. In some embodiments, the sample can be scanned in the z direction only.

In some embodiments, a scan lens (130 as shown in FIGS. 1A & 1B) can re-direct the scanning optical path to the pupil of the objective. The scan lens focuses the image of the optical axis on the scan mirror to the exit pupil of the objective. The scan lens ensures that the scanning beam remains centered on the objective, despite the distance between the scan mirror and the microscope objective, thus improving the image and light collection efficiency of the scanning beam.

Interrogation Space

An interrogation space can be thought of as an effective volume of sample in which a single molecule of interest can be detected when present. Although there are various ways to calculate the interrogation space of the sample, the simplest method for determining the effective volume (V) of the interrogation space is to calculate the effective cross section of the detection volume. Because the detection volume is typically swept through the sample by translating the detection volume through the stationary sample, the volume is typically the result of the cross sectional area of the detection volume being swept through some distance during the time of measurement. If the sample concentration (C) is known and the number of molecules detected (N) during a period of time is known, then the sample volume consists of the number of molecules detected divided by the concentration of the sample, or V=N/C (where the sample concentration has units of molecules per unit volume).

For example, in some embodiments of the system described herein, all photons detected are counted and added up in 1 msec segments (photon counting bins). If a molecule of interest is present in the 1 msec segment, the count of photons detected is typically significantly higher than background. Therefore, the distance the detection volume has moved with respect to the sample is the appropriate distance to use to calculate the volume sampled in a single segment, i.e., the interrogation space. In this example, if the sample is analyzed for 60 seconds, then effectively 60,000 segments are scanned. If the effective volume is divided by the number of segments, the resulting volume is in essence the volume of a single segment, i.e., the interrogation space. Mathematically, the volume of the single segment, i.e., the interrogation space volume (Vs), equals the number of molecules detected (N) divided by the concentration of the sample multiplied by the number of segment bins (C·n—where n represents the number of segment bins during the time the N number of molecules were counted). For exemplary purposes only, consider that a known standard of one femtomolar concentration is run through 60,000 segments, and 20 molecules of the standard are detected. Accordingly, the interrogation space volume, Vs, equals N/(C·n) or 20/(602.214.6E4), or 553.513 μm³. Thus, in this example, the interrogation space volume, which is the effective volume for one sample corresponding to one photon counting bin, is 553.513 μm³.

In addition, from the interrogation volume described previously, the cross sectional area of the sample segment can be approximated using a capillary flow system with similar optics to the disclosure described herein. The cross section area (A) is approximated by dividing the interrogation volume (Vs) by the distance (t) the detection segment moves. The distance (t) the detection segment moves is given by 2·r·s/x, where t a function of the flow rate (r), the segment bin time (s), and the cross section of the capillary (x). For exemplary purposes only, consider a bin time (s) of 1 msec, a flow rate (r) of 0.08 μL/sec, and a capillary cross sectional area (x) of 10,000 μm². Accordingly, the distance the interrogation space moves (t) is given by 2·r·s/x, or (2.0.08 μL/sec·1 msec)/(10,000 μm²), or 16.0 μm. The effective cross sectional area (A) of the detector spot can further be calculated as Vs/t, or (553.513 μm³)/(16.7 μm), or 33 μm². Note that both the value of the interrogation volume, Vs, and the cross sectional area of the interrogation volume depend on the binning time.

The lower limit on the size of the interrogation space is bounded by the wavelengths of excitation energy currently available. The upper limit of the interrogation space size is determined by the desired signal-to-noise ratios—the larger the interrogation space, the greater the noise from, e.g., Raman scattering. In some embodiments, the volume of the interrogation space is more than about 1 μm³, more than about 2 μm³, more than about 3 μm³, more than about 4 μm³, more than about 5 μm³, more than about 10 μm³, more than about 15 μm³, more than about 30 μm³, more than about 50 μm³, more than about 75 μm³, more than about 100 μm³, more than about 150 μm³, more than about 200 μm³, more than about 250 μm³, more than about 300 μm³, more than about 400 μm³, more than about 500 μm³, more than about 550 μm³, more than about 600 μm³, more than about 750 μm³, more than about 1000 μm³, more than about 2000 μm³, more than about 4000 μm³, more than about 6000 μm³, more than about 8000 μm³, more than about 10000 μm³, more than about 12000 μm³, more than about 13000 μm³, more than about 14000 μm³, more than about 15000 μm³, more than about 20000 μm³, more than about 30000 μm³, more than about 40000 μm³, or more than about 50000 μm³. In some embodiments, the interrogation space is of a volume less than about 50000 μm³, less than about 40000 μm³, less than about 30000 μm³, less than about 20000 μm³, less than about 15000 μm³, less than about 14000 μm³, less than about 13000 μm³, less than about 12000 μm³, less than about 11000 μm³, less than about 9500 μm³, less than about 8000 μm³, less than about 6500 μm³, less than about 6000 μm³, less than about 5000 μm³, less than about 4000 μm³, less than about 3000 μm³, less than about 2500 μm³, less than about 2000 μm³, less than about 1500 μm³, less than about 1000 μm³, less than about 800 μm³, less than about 600 μm³, less than about 400 μm³, less than about 200 μm³, less than about 100 μm³, less than about 75 μm³, less than about 50 μm³, less than about 25 μm³, less than about 20 μm³, less than about 15 μm³, less than about 14 μm³, less than about 13 μm³, less than about 12 μm³, less than about 11 μm³, less than about 10 μm³, less than about 5 μm³, less than about 4 μm³, less than about 3 μm³, less than about 2 μm³, or less than about 1 μm³. In some embodiments, the volume of the interrogation space is between about 1 μm³ and about 10000 μm³. In some embodiments, the interrogation space is between about 1 μm³ and about 1000 μm³. In some embodiments, the interrogation space is between about 1 μm³ and about 100 μm³. In some embodiments, the interrogation space is between about 1 μm³ and about 50 μm³. In some embodiments, the interrogation space is between about 1 μm³ and about 10 μm³. In some embodiments, the interrogation space is between about 2 μm³ and about 10 μm³. In some embodiments, the interrogation space is between about 3 μm³ and about 7 μm³.

Sample Plate

Some embodiments of the disclosure described herein use a sample plate 170 to hold the sample being detected for a single molecule of interest. The sample plate in some embodiments is a microtiter plate. The microtiter plate consists of a base 172 and a top surface 174. The top surface 174 of the microtiter plate in some embodiments consists of at least one well for containing a sample of interest. In some embodiments, the microtiter plate consists of a plurality of wells to contain a plurality of samples. The system described herein is sensitive enough so that only a small sample size is needed. In some embodiments the sample size can be less than approximately 100, 10, 1, 0.11, or 0.001 μl. The microtiter plate in some embodiments can be one constructed using microfabrication techniques. In some embodiments, the top surface of the plate can be smooth. The sample can be sized so that the sample is self-contained by the surface tension of the sample itself. In such an embodiment, the sample forms a droplet on the surface of the plate. In some embodiments, the sample can then be scanned for a molecule of interest.

The sample is scanned through the sample plate material, e.g., through the walls of the microwells. In some embodiments, the sample is scanned through the base of the sample plate. In some embodiments, the base of the sample plate is made of a material that is transparent to light. In some embodiments, the base of the sample plate is made of a material that is transparent to electromagnetic radiation. The sample plate is transparent to an excitation wavelength of interest. Using a transparent material allows the wavelength of the excitation beam to pass through the sample plate and excite the molecule of interest or the fluorescent label conjugated to the molecule of interest. The transparency of the plate further allows the detector to detect the emissions from the excited molecules of interest. In some embodiments, the base material is substantially transparent to light of wavelengths for all the wavelength associated with each of the electromagnetic radiation sources and each of the emission spectra of the labels used in multiplex single molecule analysis,

The thickness of the sample plate is also considered. The sample is scanned by an electromagnetic radiation source that passes through a portion of the material of the plate. The thickness of the plate allows an image to be formed on a first side of the portion of the plate that is scanned by a high numerical aperture lens that is positioned on a second side of the portion of the plate that is scanned. Such an embodiment facilitates the formation of an image within the sample and not within the base. The image formed corresponds to the interrogation space of the system. The image should be formed at the depth of the single molecule of interest. As previously mentioned, the thickness of the plate depends on the working distance and depth of field of the lens that is used. Commercial plates available are typically 650 microns thick.

The plate can be made out of any suitable material that allows the excitation energy to pass through the plate. In some embodiments, the plate is made of polycarbonate. In some embodiments, the plate is made of polyethylene. In some embodiments, a commercially available plate can be used, such as a NUNC™ brand plate. Any plate made of a suitable material and of a suitable thickness can be used. In preferred embodiments, the plate is made out of a material with low fluorescence, thereby reducing background fluorescence. Background fluorescence resulting from the plate material can be further avoided by minimizing the thickness of the plate.

In some embodiments, the sample consists of a small volume of fluid that can contain a particular type of molecule. In such an embodiment, the single molecule of interest, if present, can be detected and counted in a location anywhere in the fluid volume. In some embodiments, scanning the sample comprises scanning a smaller concentrated sample. In such an embodiment, the optical scanning can occur at the surface of the sample plate, for example, if the highest concentration of molecules is located at the surface of the sample plate. This can occur if the single molecules are adsorbed to the surface of the plate or if they are bound to antibodies or other binding molecules adhered to the surface of the plate. When antibodies are used to capture a single molecule of interest, the antibodies can be applied to the surface of the sample plate, e.g., to the bottom of a microwell(s). The single molecule of interest then binds to the antibodies located within the microwell. In some embodiments, an elution step is done to remove the bound single molecule of interest. The presence or absence of the unbound molecules can then be detected in a smaller sample volume. In some embodiments wherein the elution step is done, the single molecules may or may not be attached to paramagnetic beads. If no beads are used, the elution buffer can be added to the sample well and the presence or absence of the single molecule of interest can be detected. In some embodiments, a paramagnetic bead is used as a capture bead to capture the single molecule of interest.

In some embodiments of the scanning single molecule analyzer described herein, the electromagnetic (EM) radiation source is directed to the sample interrogation space without passing through the material of the sample plate. Image formation occurs in the sample on the same side as the beam directed to the sample. In such an embodiment, a water immersion lens can be used but is not required to image the sample through the air-liquid interface. In zero carryover systems wherein the objective does not come in contact with the sample, sample carryover between samples does not occur.

In some embodiments, the sample container is associated with a microfluidic cell or chip that includes appropriate sample processing reagents and binding surfaces on the chip. In some aspects, some or all of the sample processing occurs on the chip, which may be accompanied by apparatus to mobilize the sample on reagents throughout the chip (e.g., electromagnetic, pneumatic, and/or centrifugal). A sample container, well, chamber or surface that is transparent to electromagnetic radiation as described above for the plate allows for the analysis of the processed sample as described herein.

Detectors

In one embodiment, light emitted by a fluorescent label after exposure to electromagnetic radiation is detected. The emitted light can be, e.g., ultra-violet, visible or infrared. Referring to FIGS. 1A & 1B, the detector 184 (or other embodiments) can capture the amplitude and duration of photon bursts from a fluorescent moiety, and convert the amplitude and duration of the photon bursts to electrical signals. Detection devices such as CCD cameras, video input module cameras, and Streak cameras can be used to produce images with contiguous signals. Other embodiments use devices such as a bolometer, a photodiode, a photodiode array, avalanche photodiodes, and photomultipliers which produce sequential signals. Any combination of the aforementioned detectors can be used.

Several distinct characteristics of the emitted electromagnetic radiation between an interrogation space and its corresponding detector 184, can be detected including: emission wavelength, emission intensity, burst size, burst duration, and fluorescence polarization. In some embodiments, the detector 184 is a photodiode used in reverse bias. Such a photodiode set usually has an extremely high resistance. This resistance is reduced when light of an appropriate frequency shines on the P/N junction. Hence, a reverse biased diode can be used as a detector by monitoring the current running through it. Circuits based on this effect are more sensitive to light than circuits based on zero bias.

The photodiode can be provided as an avalanche photodiode. These photodiodes can be operated with much higher reverse bias than conventional photodiodes, thus allowing each photo-generated carrier to be multiplied by avalanche breakdown. This results in internal gain within the photodiode, thereby increasing the effective responsiveness and sensitivity of the device. The choice of photodiode is determined by the energy or emission wavelength emitted by the fluorescently labeled particle. In some embodiments, the detector is an avalanche photodiode detector that detects energy between 300 nm and 1700 nm. In another embodiment, silicon avalanche photodiodes can be used to detect wavelengths between 300 nm and 1100 nm. In another embodiment, the photodiode is an indium gallium arsenide photodiode that detects energy in the range of 800-2600 nm. In another embodiment, indium gallium arsenic photodiodes can be used to detect wavelengths between 900 nm and 1700 nm. In some embodiments, the photodiode is a silicon photodiode that detects energy in the range of 190-1100 nm. In another embodiment, the photodiode is a germanium photodiode that detects energy in the range of 800-1700 nm. In yet other embodiments, the photodiode is a lead sulfide photodiode that detects energy in the range of between less than 1000 nm to 3500 nm. In some embodiments, the avalanche photodiode is a single-photon detector designed to detect energy in the 400 nm to 1100 nm wavelength range. Single photon detectors are commercially available (for example Perkin Elmer and Hamamatsu).

Once a particle is labeled to render it detectable (or if the particle possesses an intrinsic characteristic rendering it detectable), any suitable detection mechanism known in the art can be used without departing from the scope of the disclosure, for example a CCD camera, a video input module camera, a Streak camera, a bolometer, a photodiode, a photodiode array, avalanche photodiodes, and photomultipliers producing sequential signals, and combinations thereof. Different characteristics of the electromagnetic radiation can be detected including: emission wavelength, emission intensity, burst size, burst duration, fluorescence polarization, and any combination thereof.

Molecules for Single Molecule Detection

The instruments, kits and methods of the disclosure can be used for the sensitive detection and determination of concentration of a number of different types of single molecules, such as markers of biological states. “Detection of a single molecule,” as that term is used herein, refers to both direct and indirect detection. For example, a single molecule can be labeled with a fluorescent label, and the molecule-label complex detected in the instruments described herein. Alternatively, a single molecule can be labeled with a fluorescent label, then the fluorescent label is detached from the single molecule, and the label detected in the instruments described herein. The term detection of a single molecule encompasses both forms of detection.

Examples of molecules or “analytes” that can be detected using the analyzer and related methods of the disclosure include: biopolymers such as proteins, nucleic acids, carbohydrates, and small molecules, both organic and inorganic. In particular, the instruments, kits, and methods described herein are useful in the detection of single molecules of proteins and small molecules in biological samples, and the determination of concentration of such molecules in the sample.

The terms “protein,” “polypeptide,” “peptide,” and “oligopeptide,” are used interchangeably herein and include any composition that includes two or more amino acids joined together by a peptide bond. It will be appreciated that polypeptides can contain amino acids other than the 20 amino acids commonly referred to as the 20 naturally occurring amino acids. Also, polypeptides can include one or more amino acids, including the terminal amino acids, which are modified by any means known in the art (whether naturally or non-naturally). Examples of polypeptide modifications include e.g., by glycosylation, or other-post-translational modification. Modifications which can be present in polypeptides of the disclosure, include, but are not limited to: acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The molecules detected by the present systems and methods can be free or can be part of a complex, e.g., an antibody-antigen complex, or more generally a protein-protein complex, e.g., complexes of troponin or complexes of prostate specific antigen (PSA).

In some embodiments, the disclosure provides compositions and methods for the sensitive detection of biological markers, and for the use of such markers in diagnosis, prognosis, and/or determination of methods of treatment.

Markers can be, for example, any composition and/or molecule or a complex of compositions and/or molecules that is associated with a biological state of an organism (e.g., a condition such as a disease or a non-disease state). A marker can be, for example, a small molecule, a polypeptide, a nucleic acid, such as DNA and RNA, a lipid, such as a phospholipid or a micelle, a cellular component such as a mitochondrion or chloroplast, etc. Markers contemplated by the disclosure can be previously known or unknown. For example, in some embodiments, the methods herein can identify novel polypeptides that can be used as markers for a biological state of interest or condition of interest, while in other embodiments, known polypeptides are identified as markers for a biological state of interest or condition. Using the systems of the disclosure it is possible that one can observe those markers, e.g., polypeptides with high potential use in determining the biological state of an organism, but that are only present at low concentrations, such as those “leaked” from diseased tissue. Other high potentially useful markers or polypeptides can be those that are related to the disease, for instance, those that are generated in the tumor-host environment. Any suitable marker that provides information regarding a biological state can be used in the methods and compositions of the disclosure. A “marker,” as that term is used herein, encompasses any molecule that can be detected in a sample from an organism and whose detection or quantitation provides information about the biological state of the organism.

Biological states include but are not limited to phenotypic states; conditions affecting an organism; states of development; age; health; pathology; disease detection, process, or staging; infection; toxicity; or response to chemical, environmental, or drug factors (such as drug response phenotyping, drug toxicity phenotyping, or drug effectiveness phenotyping).

The term “organism” as used herein refers to any living being comprised of a least one cell. An organism can be as simple as a one cell organism or as complex as a mammal. An organism of the disclosure is preferably a mammal. Such mammal can be, for example, a human or an animal such as a primate (e.g., a monkey, chimpanzee, etc.), a domesticated animal (e.g., a dog, cat, horse, etc.), farm animal (e.g., goat, sheep, pig, cattle, etc.), or laboratory animal (e.g., mouse, rat, etc.). Preferably, an organism is a human.

Labels

In some embodiments, the disclosure provides methods and compositions that include labels for the highly sensitive detection and quantitation of molecules, e.g., of markers.

Many strategies can be used for labeling target molecules to enable their detection or discrimination in a mixture of particles. The labels can be attached by any known means, including methods that utilize non-specific or specific interactions of label and target molecule. Labels can provide a detectable signal or affect the mobility of the particle in an electric field. Labeling can be accomplished directly or through binding partners.

In some embodiments, the label comprises a binding partner to the molecule of interest, where the binding partner is attached to a fluorescent moiety. The compositions and methods of the disclosure can use highly fluorescent moieties. Moieties suitable for the compositions and methods of the disclosure are described in more detail below. Fluorescent molecules may be attached to binding partners by any known means such as direct conjugation or indirectly (e.g., biotin/streptavidin).

In some embodiments, the disclosure provides a label for detecting a biological molecule comprising a binding partner for the biological molecule that is attached to a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the moiety comprises a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. In some embodiments, the moiety comprises about 2 to 4 fluorescent entities. In some embodiments, the biological molecule is a protein or a small molecule. In some embodiments, the biological molecule is a protein. The fluorescent entities can be fluorescent dye molecules. In some embodiments, the fluorescent dye molecules comprise at least one substituted indolium ring system in which the substituent on the 3-carbon of the indolium ring contains a chemically reactive group or a conjugated substance. In some embodiments, the dye molecules are ALEXA FLUOR® molecules selected from the group consisting of ALEXA FLUOR® 488, ALEXA FLUOR® 532, ALEXA FLUOR® 647, ALEXA FLUOR® 680 or ALEXA FLUOR® 700. In some embodiments, the dye molecules are ALEXA FLUOR® molecules selected from the group consisting of ALEXA FLUOR® 488, ALEXA FLUOR® 532, ALEXA FLUOR® 680 and ALEXA FLUOR® 700. Fluorescent dyes may also include Brilliant Violet™ molecules (BD Biosciences) such as Brilliant Violet 421™, Brilliant Violet 510™, Brilliant Violet 570™,|Brilliant Violet 605™, |Brilliant Violet 650™, Brilliant Violet 711™, and Brilliant Violet 785™, and ATTO™ dyes (ATTO TECH GmbH) such as ATTO™ 532. In some embodiments, the dye molecules are ALEXA FLUOR® 647 dye molecules.

In some embodiments, the binding partner comprises an antibody. In some embodiments, the antibody is a monoclonal antibody. In other embodiments, the antibody is a polyclonal antibody.

The antibody can be specific to any suitable marker. In some embodiments, the antibody is specific to a marker that is selected from the group consisting of cytokines, growth factors, oncology markers, markers of inflammation, endocrine markers, autoimmune markers, thyroid markers, cardiovascular markers, markers of diabetes, markers of infectious disease, neurological markers, respiratory markers, gastrointestinal markers, musculoskeletal markers, dermatological disorders, and metabolic markers.

Binding Partners

Any suitable binding partner with the requisite specificity for the form of molecule, e.g., a marker, to be detected can be used. If the molecule, e.g., a marker, has several different forms, various specificities of binding partners are possible. Suitable binding partners are known in the art and include antibodies, aptamers, lectins, and receptors. A useful and versatile type of binding partner is an antibody.

In some embodiments, the binding partner is an antibody specific for a molecule to be detected. The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. It will be appreciated that the choice of epitope or region of the molecule to which the antibody is raised will determine its specificity, e.g., for various forms of the molecule, if present, or for total (e.g., all, or substantially all, of the molecule).

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments that mimic antibodies can be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.). The antibody may be a monoclonal or a polyclonal antibody.

Capture binding partners and detection binding partner pairs, e.g., capture and detection antibody pairs, can be used in embodiments of the disclosure. Thus, in some embodiments, a heterogeneous assay protocol is used in which, typically, two binding partners, e.g., two antibodies, are used. One binding partner is a capture partner, usually immobilized on a solid support, and the other binding partner is a detection binding partner, typically with a detectable label attached. Such antibody pairs are available from the sources described above, e.g., BiosPacific, Emeryville, Calif. Antibody pairs can also be designed and prepared by methods well-known in the art. Compositions of the disclosure include antibody pairs wherein one member of the antibody pair is a label as described herein, and the other member is a capture antibody.

In some embodiments it is useful to use an antibody that cross-reacts with a variety of species, either as a capture antibody, a detection antibody, or both. Such embodiments include the measurement of drug toxicity by determining, e.g., release of cardiac troponin into the blood as a marker of cardiac damage. A cross-reacting antibody allows studies of toxicity to be done in one species, e.g. a non-human species, and direct transfer of the results to studies or clinical observations of another species, e.g., humans, using the same antibody or antibody pair in the reagents of the assays, thus decreasing variability between assays. Thus, in some embodiments, one or more of the antibodies for use as a binding partner to the marker of the molecule of interest, e.g., cardiac troponin, such as cardiac troponin I, can be a cross-reacting antibody. In some embodiments, the antibody cross-reacts with the marker, e.g. cardiac troponin, from at least two species selected from the group consisting of human, monkey, dog, and mouse. In some embodiments, the antibody cross-reacts with the marker, e.g., cardiac troponin, from the entire group consisting of human, monkey, dog, and mouse.

Fluorescent Moieties

In some embodiments of labels used in the disclosure, the binding partner, e.g., an antibody, is attached to a fluorescent moiety. The fluorescence of the moiety can be sufficient to allow detection in a single molecule detector, such as the single molecule detectors described herein.

A “fluorescent moiety,” as that term is used herein, includes one or more fluorescent entities whose total fluorescence is such that the moiety can be detected in the single molecule detectors described herein. Thus, a fluorescent moiety can comprise a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity can be attached to the binding partner separately or the entities can be attached together, as long as the entities as a group provide sufficient fluorescence to be detected.

Typically, the fluorescence of the moiety involves a combination of quantum efficiency and lack of photobleaching sufficient that the moiety is detectable above background levels in a single molecule detector, with the consistency necessary for the desired limit of detection, accuracy, and precision of the assay. For example, in some embodiments, the fluorescence of the fluorescent moiety is such that it allows detection and/or quantitation of a molecule, e.g., a marker, at a limit of detection of less than about 10, 5, 4, 3, 2, 1, 0.1, 0.01, 0.001, 0.00001, or 0.000001 pg/ml and with a coefficient of variation of less than about 20, 15, 14, 13, 12, 11, 10, 9, 8, 7, 6, 5, 4, 3, 2, 1% or less, e.g., about 10% or less, in the instruments described herein. In some embodiments, the fluorescence of the fluorescent moiety is such that it allows detection and/or quantitation of a molecule, e.g., a marker, at a limit of detection of less than about 5, 1, 0.5, 0.1, 0.05, 0.01, 0.005, 0.001 pg/ml and with a coefficient of variation of less than about 10%, in the instruments described herein.

“Limit of detection,” as that term is used herein, includes the lowest concentration at which one can identify a sample as containing a molecule of the substance of interest, e.g., the first non-zero value. It can be defined by the variability of zeros and the slope of the standard curve. For example, the limit of detection of an assay can be determined by running a standard curve, determining the standard curve zero value, and adding two standard deviations to that value. A concentration of the substance of interest that produces a signal equal to this value is the “lower limit of detection” concentration.

Furthermore, the moiety has properties that are consistent with its use in the assay of choice. In some embodiments, the assay is an immunoassay, where the fluorescent moiety is attached to an antibody; the moiety must not aggregate with other antibodies or proteins, or must not undergo any more aggregation than is consistent with the required accuracy and precision of the assay. In some embodiments, fluorescent moieties that are preferred are fluorescent moieties, e.g., dye molecules that have a combination of: 1) high absorption coefficient; 2) high quantum yield; 3) high photostability (low photobleaching); and 4) compatibility with labeling the molecule of interest (e.g., protein) so that it can be analyzed using the analyzers and systems of the disclosure (e.g., does not cause precipitation of the protein of interest, or precipitation of a protein to which the moiety has been attached).

Fluorescent moieties, e.g., a single fluorescent dye molecule or a plurality of fluorescent dye molecules, which are useful in some embodiments of the disclosure, can be defined in terms of their photon emission characteristics when stimulated by EM radiation. For example, in some embodiments, the disclosure utilizes a fluorescent moiety, e.g., a moiety comprising a single fluorescent dye molecule or a plurality of fluorescent dye molecules, that is capable of emitting an average of at least about 10, 20, 30, 40, 50, 75, 100, 125, 150, 175, 200, 225, 250, 275, 300, 350, 400, 500, 600, 700, 800, 900, or 1000 photons when simulated by a laser emitting light at the excitation wavelength of the moiety, where the laser is focused on a spot of not less than about 5 microns in diameter that contains the moiety, and where the total energy directed at the spot by the laser is no more than about 3 microJoules. It will be appreciated that the total energy can be achieved by many different combinations of power output of the laser and length of time of exposure of the dye moiety. E.g., a laser of a power output of 1 mW can be used for 3 ms, 3 mW for 1 ms, 6 mW for 0.5 ms, 12 mW for 0.25 ms, and so on.

In some embodiments, the fluorescent moiety comprises an average of at least about 1, 2, 3, 4, 5, 6, 7, 8, 9, or 10 fluorescent entities, e.g., fluorescent molecules. In some embodiments, the fluorescent moiety comprises an average of no more than about 2, 3, 4, 5, 6, 7, 8, 9, 10, or 11 fluorescent entities, e.g., fluorescent molecules. In some embodiments, the fluorescent moiety comprises an average of about 1 to 11 fluorescent entities. By “average” it is meant that, in a given sample that is representative of a group of labels of the disclosure, where the sample contains a plurality of the binding partner-fluorescent moiety units, the molar ratio of the particular fluorescent entity to the binding partner, as determined by standard analytical methods, corresponds to the number or range of numbers specified. For example, in embodiments wherein the label comprises a binding partner that is an antibody and a fluorescent moiety that comprises a plurality of fluorescent dye molecules of a specific absorbance, a spectrophotometric assay can be used in which a solution of the label is diluted to an appropriate level and the absorbance at 280 nm is taken to determine the molarity of the protein (antibody) and an absorbance at, e.g., 650 nm (for ALEXA FLUOR® 647), is taken to determine the molarity of the fluorescent dye molecule. The ratio of the latter molarity to the former represents the average number of fluorescent entities (dye molecules) in the fluorescent moiety attached to each antibody.

Dyes

In some embodiments, the disclosure uses fluorescent moieties that comprise fluorescent dye molecules. The dye should emit sufficient photons when stimulated by an excitation source such that it is useful in the measurement of analytes as described herein.

A non-inclusive list of useful fluorescent entities for use in the fluorescent moieties of the disclosure is given in Table 1.

TABLE 1
FLUORESCENT ENTITIES

| Dye | E Ex (nm) | E (M)−1 | Em (nm) | MMw |
|---|---|---|---|---|
| Bimane | 380 | 5,700 | 458 | 282.31 |
| Dapoxyl | 373 | 22,000 | 551 | 362.83 |
| Dimethylamino coumarin-4- | 375 | 22,000 | 470 | 344.32 |
| acetic acid | | | | |
| Marina blue | 365 | 19,000 | 460 | 367.26 |
| 8-Anilino naphthalene-1-sulfonic | 372 | | 480 | |
| acid | | | | |
| Cascade blue | 376 | 23,000 | 420 | 607.42 |
| ALEXA FLUOR ® 405 | 402 | 35,000 | 421 | 1028.26 |
| Cascade blue | 400 | 29,000 | 420 | 607.42 |
| Cascade yellow | 402 | 24,000 | 545 | 563.54 |
| BD Horizon Brilliant ™BV510 | 405 | | 510 | |
| BD Horizon Brilliant ™BV421 | 407 | 2,500,000 | 421 | 70,000 |
| Pacific blue | 410 | 46,000 | 455 | 339.21 |
| PyMPO | 415 | 26,000 | 570 | 582.41 |
| ALEXA FLUOR ® 430 | 433 | 15,000 | 539 | 701.75 |
| ATTO-425 | 438 | | 486 | |
| NBD | 465 | 22,000 | 535 | 391.34 |
| ALEXA FLUOR ® 488 | 495 | 73,000 | 519 | 643.41 |
| Fluorescein | 494 | 79,000 | 518 | 376.32 |
| Oregon Green 488 | 496 | 76,000 | 524 | 509.38 |
| Atto 495 | 495 | | 522 | |
| Cy2 | 489 | 150,000 | 506 | 713.78 |
| DY-480-XL | 500 | 40,000 | 630 | 514.60 |
| DY-485-XL | 485 | 20,000 | 560 | 502.59 |
| DY-490-XL | 486 | 27,000 | 532 | 536.58 |
| BD Horizon Brilliant ™BB515 | 490 | | 515 | |
| DY-500-XL | 505 | 90,000 | 555 | 596.68 |
| DY-520-XL | 520 | 40,000 | 664 | 514.60 |
| ALEXA FLUOR ® 532 | 531 | 81,000 | 554 | 723.77 |
| BODIPY 530/550 | 534 | 77,000 | 554 | 513.31 |
| 6-HEX | 535 | 98,000 | 556 | 680.07 |
| 6-JOE | 522 | 75,000 | 550 | 602.34 |
| Rhodamine 6G | 525 | 108,000 | 555 | 555.59 |
| Atto-520 | 520 | | 542 | |
| Cy3B | 558 | 130,000 | 572 | 658.00 |
| ALEXA FLUOR ® 610 | 612 | 138,000 | 628 | |
| ALEXA FLUOR ® 633 | 632 | 159,000 | 647 | ca. 1200 |
| ALEXA FLUOR ® 647 | 650 | 250,000 | 668 | ca. 1250 |
| BODIPY 630/650 | 625 | 101,000 | 640 | 660.50 |
| Cy5 | 649 | 250,000 | 670 | 791.99 |
| ALEXA FLUOR ® 660 | 663 | 110,000 | 690 | |
| ALEXA FLUOR ® 680 | 679 | 184,000 | 702 | |
| ALEXA FLUOR ® 700 | 702 | 192,000 | 723 | |
| ALEXA FLUOR ® 750 | 749 | 240,000 | 782 | |
| B-phycoerythrin | 546, 565 | 2,410,000 | 575 | 240,000 |
| R-phycoerythrin | 480, 546, 565 | 1,960,000 | 578 | 240,000 |
| Allophycocyanin | 650 | 700,000 | 660 | 700,000 |
| PBXL-1 | 545 | | 666 | |
| PBXL-3 | 614 | | 662 | |

Atto-tec dyes

| Name | Ex (nm) | Em (nm) | QY | □ (ns) |
|---|---|---|---|---|
| ATTO 425 | 436 | 486 | 0.9 | 3.5 |
| ATTO 495 | 495 | 522 | 0.45 | 2.4 |
| ATTO 520 | 520 | 542 | 0.9 | 3.6 |
| ATTO 532 | 532 | 553 | 0.9 | 3.8 |
| ATTO 560 | 561 | 585 | 0.92 | 3.4 |
| ATTO 590 | 598 | 634 | 0.8 | 3.7 |
| ATTO 610 | 605 | 630 | 0.7 | 3.3 |
| ATTO 655 | 665 | 690 | 0.3 | 1.9 |
| ATTO 680 | 680 | 702 | 0.3 | 1.8 |

Dyomics Fluors

| | | Molar absorbance* | | Molecular weight |
|---|---|---|---|---|
| label | Ex (nm) | [1 · mol−1 · cm−1] | Em (nm) | #[g · mol−1] |
| DY-495/5 | 495 | 70,000 | 520 | 489.47 |
| DY-495/6 | 495 | 70,000 | 520 | 489.47 |
| DY-495X/5 | 495 | 70,000 | 520 | 525.95 |
| DY-495X/6 | 495 | 70,000 | 520 | 525.95 |
| DY-505/5 | 505 | 85,000 | 530 | 485.49 |
| DY-505/6 | 505 | 85,000 | 530 | 485.49 |
| DY-505X/5 | 505 | 85,000 | 530 | 523.97 |
| DY-505X/6 | 505 | 85,000 | 530 | 523.97 |
| DY-550 | 553 | 122,000 | 578 | 667.76 |
| DY-555 | 555 | 100.000 | 580 | 636.18 |
| DY-610 | 609 | 81.000 | 629 | 667.75 |
| DY-615 | 621 | 200.000 | 641 | 578.73 |
| DY-630 | 636 | 200.000 | 657 | 634.84 |
| DY-631 | 637 | 185.000 | 658 | 736.88 |
| DY-633 | 637 | 180.000 | 657 | 751.92 |
| DY-635 | 647 | 175.000 | 671 | 658.86 |
| DY-636 | 645 | 190.000 | 671 | 760.91 |
| DY-650 | 653 | 170.000 | 674 | 686.92 |
| DY-651 | 653 | 160.000 | 678 | 888.96 |
| DYQ-660 | 660 | 117,000 | — | 668.86 |
| DYQ-661 | 661 | 116,000 | — | 770.90 |
| DY-675 | 674 | 110.000 | 699 | 706.91 |
| DY-676 | 674 | 145.000 | 699 | 807.95 |
| DY-680 | 690 | 125.000 | 709 | 634.84 |
| DY-681 | 691 | 125.000 | 708 | 736.88 |
| DY-700 | 702 | 96.000 | 723 | 668.86 |
| DY-701 | 706 | 115.000 | 731 | 770.90 |
| DY-730 | 734 | 185.000 | 750 | 660.88 |
| DY-731 | 736 | 225.000 | 759 | 762.92 |
| DY-750 | 747 | 240.000 | 776 | 712.96 |
| DY-751 | 751 | 220.000 | 779 | 814.99 |
| DY-776 | 771 | 147.000 | 801 | 834.98 |
| DY-780-OH | 770 | 70.000 | 810 | 757.34 |
| DY-780-P | 770 | 70.000 | 810 | 957.55 |
| DY-781 | 783 | 98.000 | 800 | 762.92 |
| DY-782 | 782 | 102.000 | 800 | 660.88 |
| EVOblue-10 | 651 | 101.440 | 664 | 389.88 |
| EVOblue-30 | 652 | 102.000 | 672 | 447.51 |

Quantum Dots: Qdot 525, QD 565, QD 585, QD 605, QD 655, QD 705, QD 800

Suitable dyes for use in the disclosure include modified carbocyanine dyes. On such modification comprises modification of an indolium ring of the carbocyanine dye to permit a reactive group or conjugated substance at the number three position. The modification of the indolium ring provides dye conjugates that are uniformly and substantially more fluorescent on proteins, nucleic acids and other biopolymers, than conjugates labeled with structurally similar carbocyanine dyes bound through the nitrogen atom at the number one position. In addition to having more intense fluorescence emission than structurally similar dyes at virtually identical wavelengths, and decreased artifacts in their absorption spectra upon conjugation to biopolymers, the modified carbocyanine dyes have greater photostability and higher absorbance (extinction coefficients) at the wavelengths of peak absorbance than the structurally similar dyes. Thus, the modified carbocyanine dyes result in greater sensitivity in assays using the modified dyes and their conjugates. Preferred modified dyes include compounds that have at least one substituted indolium ring system in which the substituent on the 3-carbon of the indolium ring contains a chemically reactive group or a conjugated substance. Other dye compounds include compounds that incorporate an azabenzazolium ring moiety and at least one sulfonate moiety. The modified carbocyanine dyes that can be used to detect individual molecules in various embodiments of the disclosure are described in U.S. Pat. No. 6,977,305, which is herein incorporated by reference in its entirety. Thus, in some embodiments the labels of the disclosure utilize a fluorescent dye that includes a substituted indolium ring system in which the substituent on the 3-carbon of the indolium ring contains a chemically reactive group or a conjugated substance group.

Currently available organic fluors can be improved by rendering them less hydrophobic by adding hydrophilic groups such as polyethylene. Alternatively, currently sulfonated organic fluors such as the ALEXA FLUOR® 647 dye can be rendered less acidic by making them zwitterionic. Particles such as antibodies that are labeled with the modified fluors are less likely to bind non-specifically to surfaces and proteins in immunoassays, and thus enable assays that have greater sensitivity and lower backgrounds. Methods for modifying and improving the properties of fluorescent dyes for the purpose of increasing the sensitivity of a system that detects single molecules are known in the art. Preferably, the modification improves the Stokes shift while maintaining a high quantum yield.

Quantum Dots

In some embodiments, the fluorescent label moiety that is used to detect a molecule in a sample using the analyzer systems of the disclosure is a quantum dot. Quantum dots (QDs), also known as semiconductor nanocrystals or artificial atoms, are semiconductor crystals that contain anywhere between 100 to 1,000 electrons and range from 2-10 nm. Some QDs can be between 10-20 nm in diameter. QDs have high quantum yields, which makes them particularly useful for optical applications. QDs are fluorophores that fluoresce by forming excitons, which are similar to the excited state of traditional fluorophores, but have much longer lifetimes of up to 200 nanoseconds. This property provides QDs with low photobleaching. The energy level of QDs can be controlled by changing the size and shape of the QD, and the depth of the QDs' potential. One optical features of small excitonic QDs is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the QD. Larger QDs have more energy levels which are more closely spaced, thus allowing the QD to absorb photons containing less energy, i.e., those closer to the red end of the spectrum. Because the emission frequency of a dot is dependent on the bandgap, it is possible to control the output wavelength of a dot with extreme precision. In some embodiments the protein that is detected with the single molecule analyzer system is labeled with a QD. In some embodiments, the single molecule analyzer is used to detect a protein labeled with one QD and using a filter to allow for the detection of different proteins at different wavelengths.

QDs have broad excitation and narrow emission properties which, when used with color filtering, require only a single electromagnetic source to resolve individual signals during multiplex analysis of multiple targets in a single sample. Thus, in some embodiments, the analyzer system comprises one continuous wave laser and particles that are each labeled with one QD. Colloidally prepared QDs are free floating and can be attached to a variety of molecules via metal coordinating functional groups. These groups include but are not limited to thiol, amine, nitrile, phosphine, phosphine oxide, phosphonic acid, carboxylic acids or other ligands. By bonding appropriate molecules to the surface, the quantum dots can be dispersed or dissolved in nearly any solvent or incorporated into a variety of inorganic and organic films. Quantum dots (QDs) can be coupled to streptavidin directly through a maleimide ester coupling reaction or to antibodies through a meleimide-thiol coupling reaction. This yields a material with a biomolecule covalently attached on the surface, which produces conjugates with high specific activity. In some embodiments, the protein that is detected with the single molecule analyzer is labeled with one quantum dot. In some embodiments, the quantum dot is between 10 and 20 nm in diameter. In other embodiments, the quantum dot is between 2 and 10 nm in diameter. In other embodiments, the quantum dot is about 2 nm, 3 nm, 4 nm, 5 nm, 6 nm, 7 nm, 8 nm, 9 nm, 10 nm, 11 nm, 12 nm, 13 nm, 14 nm, 15 v, 16 nm, 17 nm, 18 nm, 19 nm or 20 nm in diameter. Useful Quantum Dots comprise QD 605, QD 610, QD 655, and QD 705. A preferred Quantum Dot is QD 605.

Polymeric Fluorophores

In some embodiments, the fluorescent label moiety is a polymeric fluorophore. Polymeric fluorophores are designed to have greater absorption of excitation light and brighter emission fluorescence than convention fluorophores. Polymeric fluorophores work as molecular antennae and gather higher levels of excitation energy. This energy can either be emitted by the polymer itself as fluorescence or can be transferred to a covalently linked tandem fluorescent dye through a fluorescence resonance energy transfer (FRET) process.

Polymeric fluorophores can be designed such that they have a polymeric backbone that has instrinsic absorption and fluorescence at a specific wavelength such as BD Horizon™ BV421 that is excited at 407 nm and maximally fluoresces at 421 nm and BD Horizon™ BB515 that is excited at 490 nm and maximally fluoresces at 515 nm. Tandem dyes can also be created where acceptor dyes are covalently linked to the polymeric backbone to allow multiple emission spectra besides the intrinsic polymeric one. This allows for a family of fluorophores that can all be excited at the same wavelength but emit at different wavelengths. In some embodiments the protein that is detected with the single molecule analyzer system is labeled with a polymeric fluorophore. In some embodiments, the protein that is detected with the single molecule analyzer system is labeled with biotin and a streptavidin molecule covalently bound to a polymeric fluorophore is added which binds to the biotin. In some embodiments, the single molecule analyzer is used to detect a protein labeled with a polymeric fluorophore.

Some polymeric fluorophores have been designed to have a narrow excitation and multiple potential emission properties that depend on covalently linked fluorophores. These fluorophores, when used with color filtering, require only a single electromagnetic excitation source to resolve individual signals during multiplex analysis of multiple targets in a single sample. Thus, in some embodiments, the analyzer system comprises one continuous wave laser and particles that are each labeled with a different polymeric fluorophore, which may be detected by multiple detectors. Polymeric fluorophores can be coupled to antibodies directly or to antibodies indirectly through a coupling reaction (e.g., biotin/streptavin). This yields a material with a biomolecule covalently attached on the surface, which produces conjugates with high specific activity. In some embodiments, the protein that is detected with the single molecule analyzer is labeled with one polymeric fluorophore. Useful polymeric fluorophores comprise BV421, BV510, and BB515.

Binding Partner-Fluorescent Moiety Compositions

The labels of the disclosure generally contain a binding partner, e.g., an antibody, bound to a fluorescent moiety to provide the requisite fluorescence for detection and quantitation in the instruments described herein. Any suitable combination of binding partner and fluorescent moiety for detection in the single molecule detectors described herein can be used as a label in the disclosure. In some embodiments, the disclosure provides a label for a marker of a biological state, where the label includes an antibody to the marker and a fluorescent moiety. The marker can be any of the markers described above. The antibody can be any antibody as described above. A fluorescent moiety can be attached such that the label is capable of emitting an average of at least about 50, 75, 100, 125, 150, 175, 200, 225, 250, 275, 300, 350, 400, 500, 600, 700, 800, 900, or 1000 photons when simulated by a laser emitting light at the excitation wavelength of the moiety, where the laser is focused on a spot of not less than about 5 microns in diameter that contains the label, and wherein the total energy directed at the spot by the laser is no more than about 3 microJoules.

Attachment of the fluorescent moiety, or fluorescent entities that make up the fluorescent moiety, to the binding partner, e.g., an antibody, can be by any suitable means; such methods are well-known in the art and exemplary methods are given in the Examples. In some embodiments, after attachment of the fluorescent moiety to the binding partner to form a label for use in the methods of the disclosure, and prior to the use of the label for labeling the marker of interest, it is useful to perform a filtration step. E.g., an antibody-dye label can be filtered prior to use, e.g., through a 0.2 micron filter, or any suitable filter for removing aggregates. Other reagents for use in the assays of the disclosure can also be filtered, e.g., through a 0.2 micron filter, or any suitable filter. Without being bound by theory, it is thought that such filtration removes a portion of the aggregates of the, e.g., antibody-dye labels. Such aggregates can bind as a unit to the protein of interest, but, upon release in elution buffer, the aggregates are likely to disaggregate. Therefore false positives can result when several labels are detected from an aggregate that has bound to only a single protein molecule of interest. Regardless of theory, filtration has been found to reduce false positives in the subsequent assay and to improve accuracy and precision.

It will be appreciated that immunoassays often employ a sandwich format in which binding partner pairs, e.g. antibodies, to the same molecule, e.g., a marker, are used. The disclosure also encompasses binding partner pairs, e.g., antibodies, wherein both antibodies are specific to the same molecule, e.g., the same marker, and wherein at least one member of the pair is a label as described herein. Thus, for any label that includes a binding-partner and a fluorescent moiety, the disclosure also encompasses a pair of binding partners wherein the first binding partner, e.g., an antibody, is part of the label, and the second binding partner, e.g., an antibody, is, typically, unlabeled and serves as a capture binding partner. In addition, binding partner pairs are frequently used in FRET assays. FRET assays useful in the disclosure are disclosed in U.S. patent application publication No. US20060078998, and the disclosure also encompasses binding partner pairs, each of which includes a FRET label.

Highly Sensitive Analysis of Molecules

In one aspect, the disclosure provides a method for determining the presence or absence of a single molecule, e.g., a molecule of a marker, in a sample, by: i) labeling the molecule if present, with a label; and ii) detecting the presence or absence of the label, wherein the detection of the presence of the label indicates the presence of the single molecule in the sample. In some embodiments, the method is capable of detecting the molecule at a limit of detection of less than about 100, 80, 60, 50, 40, 30, 20, 15, 12, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001 femtomolar. Detection limits can be determined by use of an appropriate standard, e.g., National Institute of Standards and Technology reference standard material.

The methods also provide methods of determining a concentration of a molecule, e.g., a marker indicative of a biological state, in a sample by detecting single molecules of the molecule in the sample. The “detecting” of a single molecule includes detecting the molecule directly or indirectly. In the case of indirect detection, labels that correspond to single molecules, e.g., labels attached to the single molecules, can be detected.

In some embodiments, the disclosure provides a method for determining the presence or absence of a single molecule of a protein in a biological sample, comprising labeling the molecule with a label and detecting the presence or absence of the label in a single molecule detector, wherein the label comprises a fluorescent moiety that is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. The single molecule detector may, in some embodiments, comprise not more than one interrogation space. The limit of detection of the single molecule in the sample can be less than about 10, 1, 0.1, 0.01, or 0.001 femtomolar. In some embodiments, the limit of detection is less than about 1 femtomolar. The detecting can comprise detecting electromagnetic radiation emitted by the fluorescent moiety. The method can further comprise exposing the fluorescent moiety to electromagnetic radiation, e.g., electromagnetic radiation provided by a laser, such as a laser with a power output of about 2, 3, 4, 5, 6, 7, 8, 9, 10, 11, 12, 13, 14, 15, 16, 17, 18, 19, or 20 mW. In some embodiments, the laser stimulus provides light to the interrogation space for between about 10 to 1000 microseconds, or about 1000, 250, 100, 50, 25 or 10 microseconds. In some embodiments, the label further comprises a binding partner specific for binding the molecule, such as an antibody.

In some embodiments, detecting the presence or absence of the label comprises: (i) directing electromagnetic radiation from an electromagnetic radiation source to an interrogation space; (ii) providing electromagnetic radiation that is sufficient to stimulate the label, such as a fluorescent moiety, to emit photons if the label is present in the interrogation space; (iii) translating the interrogation space through the sample thereby moving the interrogation space to detect the presence or absence of other single molecules; and (iv) detecting photons emitted during the exposure of step (ii). The method can further comprise determining a background photon level in the interrogation space, wherein the background level represents the average photon emission of the interrogation space when it is subjected to electromagnetic radiation in the same manner as in step (ii), but without label in the interrogation space. The method can further comprise comparing the amount of photons detected in step (iv) to a threshold photon level, wherein the threshold photon level is a function of the background photon level, wherein an amount of photons detected in step (iv) greater that the threshold level indicates the presence of the label, and an amount of photons detected in step (iv) equal to or less than the threshold level indicates the absence of the label.

Sample

The sample can be any suitable sample. Typically, the sample is a biological sample, e.g., a biological fluid. Such fluids include, without limitation, bronchoalveolar lavage fluid (BAL), blood, serum, plasma, urine, nasal swab, cerebrospinal fluid, pleural fluid, synovial fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues including biopsies of normal, malignant, and suspect tissues or any other constituents of the body which can contain the target particle of interest. Other similar specimens such as cell or tissue culture or culture broth are also of interest.

In some embodiments, the sample is a blood sample. In some embodiments the sample is a plasma sample. In some embodiments the sample is a serum sample. In some embodiments, the sample is a urine sample. In some embodiments, the sample is a nasal swab.

Sample Preparation

In general, any method of sample preparation can be used that produces a label corresponding to a molecule of interest, e.g., a marker of a biological state to be measured, where the label is detectable in the instruments described herein. As is known in the art, sample preparation in which a label is added to one or more molecules can be performed in a homogeneous or heterogeneous format. In some embodiments, the sample preparation is formed in a homogenous format. In analyzer systems employing a homogenous format, unbound label is not removed from the sample. See, e.g., US 20060078998. In some embodiments, the particle or particles of interest are labeled by addition of labeled antibody or antibodies that bind to the particle or particles of interest.

In some embodiments, a heterogeneous assay format is used, wherein, typically, a step is employed for removing unbound label. Such assay formats are well-known in the art. One particularly useful assay format is a sandwich assay, e.g., a sandwich immunoassay. In this format, the molecule of interest, e.g., a marker of a biological state, is captured, e.g., on a solid support, using a capture binding partner. Unwanted molecules and other substances can then optionally be washed away, followed by binding of a label comprising a detection binding partner and a detectable label, e.g., a fluorescent moiety. Further washes remove unbound label, then the detectable label is released, usually though not necessarily still attached to the detection binding partner. In alternative embodiments, sample and label are added to the capture binding partner without a wash in between, e.g., at the same time. Other variations will be apparent to one of skill in the art.

In some embodiments, the method for detecting the molecule of interest, e.g., a marker of a biological state, uses a sandwich assay with antibodies, e.g., monoclonal antibodies, as capture binding partners. The method comprises binding molecules in a sample to a capture antibody that is immobilized on a binding surface, and binding the label comprising a detection antibody to the molecule to form a “sandwich” complex. The label comprises the detection antibody and a fluorescent moiety, as described herein, which is detected, e.g., using the single molecule analyzers of the disclosure. Both the capture and detection antibodies specifically bind the molecule. Many examples of sandwich immunoassays are known, and some are described in U.S. Pat. No. 4,168,146 to Grubb et al. and U.S. Pat. No. 4,366,241 to Tom et al., both of which are incorporated herein by reference. Further examples specific to specific markers are described in the Examples.

The capture binding partner can be attached to a solid support, e.g., a microtiter plate or paramagnetic beads. In some embodiments, the disclosure provides a binding partner for a molecule of interest, e.g., a marker of a biological state, attached to a paramagnetic bead. Any suitable binding partner that is specific for the molecule that it is wished to capture can be used. The binding partner can be an antibody, e.g., a monoclonal antibody. Production and sources of antibodies are described elsewhere herein. It will be appreciated that antibodies identified herein as useful as a capture antibody can also be useful as detection antibodies, and vice versa.

The attachment of the binding partner, e.g., an antibody, to the solid support can be covalent or noncovalent. In some embodiments, the attachment is noncovalent. An example of a noncovalent attachment well-known in the art is that between biotin-avidin and streptavidin. Thus, in some embodiments, a solid support, e.g., a microtiter plate or a paramagnetic bead, is attached to the capture binding partner, e.g., an antibody, through noncovalent attachment, e.g., biotin-avidin/streptavidin interactions. In some embodiments, the attachment is covalent. Thus, in some embodiments, a solid support, e.g., a microtiter plate or a paramagnetic bead, is attached to the capture binding partner, e.g., an antibody, through covalent attachment.

The capture antibody can be covalently attached in an orientation that optimizes the capture of the molecule of interest. For example, in some embodiments, a binding partner, e.g., an antibody, is attached in a orientated manner to a solid support, e.g., a microtiter plate or a paramagnetic microparticle.

An exemplary protocol for oriented attachment of an antibody to a solid support is as follows. IgG is dissolved in 0.1 M sodium acetate buffer, pH 5.5 to a final concentration of 1 mg/ml. An equal volume of ice cold 20 mM sodium periodate in 0.1 M sodium acetate, pH 5.5 is added. The IgG is allowed to oxidize for ½ hour on ice. Excess periodate reagent is quenched by the addition of 0.15 volume of 1 M glycerol. Low molecular weight byproducts of the oxidation reaction are removed by ultrafiltration. The oxidized IgG fraction is diluted to a suitable concentration (typically 0.5 mg/ml IgG) and reacted with hydrazide-activated multiwell plates for at least two hours at room temperature. Unbound IgG is removed by washing the multiwell plate with borate buffered saline or another suitable buffer. The plate can be dried for storage if desired. A similar protocol can be followed to attach antibodies to microbeads if the material of the microbead is suitable for such attachment.

In some embodiments, the solid support is a microtiter plate. In some embodiments, the solid support is a paramagnetic bead. An exemplary paramagnetic bead is Streptavidin C1 (Dynal, 650.01-03). Other suitable beads will be apparent to those of skill in the art. Methods for attachment of antibodies to paramagnetic beads are well-known in the art. One example is given in Example 2.

The molecule of interest is contacted with the capture binding partner, e.g., capture antibody immobilized on a solid support. Some sample preparation can be used, e.g., preparation of serum from blood samples or concentration procedures before the sample is contacted with the capture antibody. Protocols for binding of proteins in immunoassays are well-known in the art.

The time allowed for binding will vary depending on the conditions; it will be apparent that shorter binding times are desirable in some settings, especially in a clinical setting. The use of, e.g., paramagnetic beads can reduce the time required for binding. In some embodiments, the time allowed for binding of the molecule of interest to the capture binding partner, e.g., an antibody, is less that about 12, 10, 8, 6, 4, 3, 2, or 1 hours, or less than about 60, 50, 40, 30, 25, 20, 15, 10, or 5 minutes.

In some embodiments, following the binding of particles of the molecule of interest to the capture binding partner, e.g., a capture antibody, particles that bound nonspecifically, as well as other unwanted substances in the sample, are washed away leaving substantially only specifically bound particles of the molecule of interest. In other embodiments, no wash is used between additions of sample and label, which can reduce sample preparation time. Thus, in some embodiments, the time allowed for both binding of the molecule of interest to the capture binding partner, e.g., an antibody, and binding of the label to the molecule of interest, is less that about 12, 10, 8, 6, 4, 3, 2, or 1 hours, or less than about 60, 50, 40, 30, 25, 20, 15, 10, or 5 minutes.

Some immunoassay diagnostic reagents, including the capture and signal antibodies used to measure the molecule of interest, can be derived from animal sera. Endogenous human heterophilic antibodies, or human anti-animal antibodies, which have the ability to bind to immunoglobulins of other species, are present in the serum or plasma of more than 10% of patients. These circulating heterophilic antibodies can interfere with immunoassay measurements. In sandwich immunoassays, these heterophilic antibodies can either bridge the capture and detection (diagnostic) antibodies, thereby producing a false-positive signal, or they can block the binding of the diagnostic antibodies, thereby producing a false-negative signal. In competitive immunoassays, the heterophilic antibodies can bind to the analytic antibody and inhibit its binding to the molecule of interest. They can also either block or augment the separation of the antibody-molecule of interest complex from free molecule of interest, especially when antispecies antibodies are used in the separation systems. Therefore, the impact of these heterophilic antibody interferences is difficult to predict and it can be advantageous to block the binding of heterophilic antibodies. In some embodiments of the disclosure, the immunoassay includes the step of depleting the sample of heterophilic antibodies using one or more heterophilic antibody blockers. Embodiments of the methods of the disclosure contemplate preparing the sample prior to analysis with the single molecule detector. The appropriateness of the method of pretreatment can be determined. Biochemicals to minimize immunoassay interference caused by heterophilic antibodies are commercially available. In some embodiments the heterophilic antibody can be immunoextracted from the sample using methods known in the art.

Label is added either with or following the addition of sample and washing. Protocols for binding antibodies and other immunolabels to proteins and other molecules are well-known in the art. If the label binding step is separate from that of capture binding, the time allowed for label binding can be important, e.g., in clinical applications or other time sensitive settings. In some embodiments, the time allowed for binding of the molecule of interest to the label, e.g., an antibody-dye, is less than about 12, 10, 8, 6, 4, 3, 2, or 1 hours, or less than about 60, 50, 40, 30, 25, 20, 15, 10, or 5 minutes. Excess label is removed by washing.

In some embodiments, the label is not eluted from the protein of interest. In other embodiments, the label is eluted from the protein of interest. Preferred elution buffers are effective in releasing the label without generating significant background. It is useful if the elution buffer is bacteriostatic. Elution buffers used in the disclosure can comprise a chaotrope, a buffer, an albumin to coat the surface of the microtiter plate, and a surfactant selected so as to produce a relatively low background. The chaotrope can comprise urea, a guanidinium compound, or other useful chaotropes. The buffer can comprise borate buffered saline, or other useful buffers. The protein carrier can comprise, e.g., an albumin, such as human, bovine, or fish albumin, an IgG, or other useful carriers. The surfactant can comprise an ionic or nonionic detergent including Tween 20, Triton X-100, sodium dodecyl sulfate (SDS), and others.

In another embodiment, the solid phase binding assay can be a competitive binding assay. One such method is as follows. First, a capture antibody immobilized on a binding surface is competitively bound by i) a molecule of interest, e.g., marker of a biological state, in a sample, and ii) a labeled analog of the molecule comprising a detectable label (the detection reagent). Second, the amount of the label using a single molecule analyzer is measured. Another such method is as follows. First, an antibody having a detectable label (the detection reagent) is competitively bound to i) a molecule of interest, e.g., marker of a biological state in a sample, and ii) an analog of the molecule that is immobilized on a binding surface (the capture reagent). Second, the amount of the label using a single molecule analyzer is measured. An “analog of a molecule” refers, herein, to a species that competes with a molecule for binding to a capture antibody. Examples of competitive immunoassays are disclosed in U.S. Pat. No. 4,235,601 to Deutsch et al., U.S. Pat. No. 4,442,204 to Liotta, and U.S. Pat. No. 5,208,535 to Buechler et al., all of which are incorporated herein by reference.

Detection of Molecule of Interest and Determination of Concentration

Following elution, the presence or absence of the label in the sample is detected using a single molecule detector. A sample can contain no label, a single label, or a plurality of labels. The number of labels corresponds to or is proportional to (if dilutions or fractions of samples are used) the number of molecules of the molecule of interest, e.g., a marker of a biological state captured during the capture step.

Any suitable single molecule detector capable of detecting the label used with the molecule of interest can be used, including scanning analyzer system 100. Suitable single molecule detectors are described herein. Typically the detector is part of a system that includes an automatic sampler for sampling prepared samples, and, optionally, a recovery system to recover samples.

In some embodiments, the sample is analyzed in a single molecule analyzer that uses a laser to illuminate an interrogation space containing a sample, a detector to detect radiation emitted from the interrogation space, and a scan motor and mirror attached to the motor to translate the interrogation space through the sample. In some embodiments, the single molecule analyzer further comprises a microscope objective lens that collects light emitted from the sample as the interrogation space is translated through the sample, e.g., a high numerical aperture microscope objective. In some embodiments, the laser and detector are configured in a confocal arrangement. In some embodiments, the laser is a continuous wave laser. In some embodiments, the detector is an avalanche photodiode detector. In some embodiments, the interrogation space is translated through the sample using a mirror attached to the scan motor. In some embodiments, the interrogation space is translated through the sample using multiple mirrors or a prism attached to the scan motor. In some embodiments, the disclosure provides an analyzer system that includes a sampling system capable of automatically sampling a plurality of samples with zero carryover between subsequently measured samples.

In some embodiments, the single molecule detector used in the methods of the disclosure uses a sample plate, a continuous wave laser directed toward a sample plate in which the sample is contained, a high numerical aperture microscope objective lens that collects light emitted from the sample as interrogation space is translated through the sample, wherein the lens has a numerical aperture of at least about 0.8, an avalanche photodiode detector to detect radiation emitted from the interrogation space, and a scan motor with a moveable mirror to translate the interrogation space through the sample wherein the interrogation space is between about 1 μm³ and about 10000 μm³.

In some embodiments, the single molecule detector is capable of determining a concentration for a molecule of interest in a sample wherein the sample can range in concentration over a range of at least about 100-fold, 1000-fold, 10,000-fold, 100,000-fold, 300,000-fold, 1,000,000-fold, 10,000,000-fold, or 30,000,000-fold. In some embodiments, the methods of the disclosure use a single molecule detector capable detecting a difference of less than about 50%, 40%, 30%, 20%, 15%, or 10% in concentration of an analyte between a first sample and a second sample contained in a sample plate, wherein the volume of the first sample and the second sample introduced into the analyzer is less than about 100, 90, 80, 70, 60, 50, 40, 30, 20, 15, 10, 5, 4, 3, 2, or 1 and wherein the analyte is present at a concentration of less than about 100, 90, 80, 70, 60, 50, 40, 30, 20, 15, 10, 5, 4, 3, 2, or 1 femtomolar.

A feature that contributes to the extremely high sensitivity of the instruments and methods of the disclosure is the method of detecting and counting molecules. Briefly, the sample contained in the sample plate is effectively divided into a series of detection events, by translating an interrogation space through the sample plate wherein EM radiation from a laser for a predetermined period of time is directed to the wavelength, and photons emitted during that time are detected. In some embodiments where labels are used, the wavelength of the EM radiation may be chosen as an appropriate excitation wavelength for the fluorescent moiety used in the label. Each predetermined period of time is a “bin.” If the total number of photons detected in a given bin exceeds a predetermined threshold level, a detection event (“DE”) is registered for that bin, i.e., a label has been detected. A detection event can also be thought of as each “flash” of light that is brighter than the threshold. If the total number of photons is not at the predetermined threshold level, no detection event is registered.

In some embodiments, the processing sample concentration is dilute enough that, for a large percentage of detection events, the detection event represents only one label passing through the window, which corresponds to a single molecule of interest in the original sample. Accordingly, few detection events represent more than one label in a single bin. However, as the concentration goes up, the probability that two molecules will transit the detector at the same time (in the same counting bin) becomes significant. In this case, one flash of light represents two (or more) molecules. In some embodiments, further refinements are applied to allow greater concentrations of label in the processing sample to be detected accurately, i.e., concentrations at which the probability of two or more labels being detected as a single detection event is no longer insignificant. To detect single molecules at greater concentrations, the number of photons detected over a threshold level is counted. In other words, the brightness of each flash is measured. The sum of the photon counts is called event photons (“EP”).

Although other bin times can be used without departing from the scope of the disclosure, in some embodiments the bin times are selected in the range of about 1 microsecond to about 5 ms. In some embodiments, the bin time is more than about 1, 2, 3, 4, 5, 6, 7, 8, 9, 10, 15, 20, 30, 40, 50, 60, 70, 80, 90, 100, 200, 250, 300, 400, 500, 600, 700, 750, 800, 900, 1000, 2000, 3000, 4000, or 5000 microseconds. In some embodiments, the bin time is less than about 2, 3, 4, 5, 6, 7, 8, 9, 10, 15, 20, 30, 40, 50, 60, 70, 80, 90, 100, 200, 250, 300, 400, 500, 600, 700, 750, 800, 900, 1000, 2000, 3000, 4000, or 5000 microseconds. In some embodiments, the bin time is about 1 to 1000 microseconds. In some embodiments, the bin time is about 1 to 750 microseconds. In some embodiments, the bin time is about 1 to 500 microseconds. In some embodiments, the bin time is about 1 to 250 microseconds. In some embodiments, the bin time is about 1 to 100 microseconds. In some embodiments, the bin time is about 1 to 50 microseconds. In some embodiments, the bin time is about 1 to 40 microseconds. In some embodiments, the bin time is about 1 to 30 microseconds. In some embodiments, the bin time is about 1 to 25 microseconds. In some embodiments, the bin time is about 1 to 20 microseconds. In some embodiments, the bin time is about 1 to 10 microseconds. In some embodiments, the bin time is about 1 to 7.5 microseconds. In some embodiments, the bin time is about 1 to 5 microseconds. In some embodiments, the bin time is about 5 to 500 microseconds. In some embodiments, the bin time is about 5 to 250 microseconds. In some embodiments, the bin time is about 5 to 100 microseconds. In some embodiments, the bin time is about 5 to 50 microseconds. In some embodiments, the bin time is about 5 to 20 microseconds. In some embodiments, the bin time is about 5 to 10 microseconds. In some embodiments, the bin time is about 10 to 500 microseconds. In some embodiments, the bin time is about 10 to 250 microseconds. In some embodiments, the bin time is about 10 to 100 microseconds. In some embodiments, the bin time is about 10 to 50 microseconds. In some embodiments, the bin time is about 10 to 30 microseconds. In some embodiments, the bin time is about 10 to 20 microseconds. In some embodiments, the bin time is about 1 microsecond. In some embodiments, the bin time is about 2 microseconds. In some embodiments, the bin time is about 3 microseconds. In some embodiments, the bin time is about 4 microseconds. In some embodiments, the bin time is about 5 microseconds. In some embodiments, the bin time is about 6 microseconds. In some embodiments, the bin time is about 7 microseconds. In some embodiments, the bin time is about 8 microseconds. In some embodiments, the bin time is about 9 microseconds. In some embodiments, the bin time is about 10 microseconds. In some embodiments, the bin time is about 11 microseconds. In some embodiments, the bin time is about 12 microseconds. In some embodiments, the bin time is about 13 microseconds. In some embodiments, the bin time is about 14 microseconds. In some embodiments, the bin time is about 5 microseconds. In some embodiments, the bin time is about 15 microseconds. In some embodiments, the bin time is about 16 microseconds. In some embodiments, the bin time is about 17 microseconds. In some embodiments, the bin time is about 18 microseconds. In some embodiments, the bin time is about 19 microseconds. In some embodiments, the bin time is about 20 microseconds. In some embodiments, the bin time is about 25 microseconds. In some embodiments, the bin time is about 30 microseconds. In some embodiments, the bin time is about 40 microseconds. In some embodiments, the bin time is about 50 microseconds. In some embodiments, the bin time is about 100 microseconds. In some embodiments, the bin time is about 250 microseconds. In some embodiments, the bin time is about 500 microseconds. In some embodiments, the bin time is about 750 microseconds. In some embodiments, the bin time is about 1000 microseconds.

In some embodiments, determining the concentration of a particle-label complex in a sample comprises determining the background noise level. In some embodiments, the background noise level is determined from the mean noise level, or the root-mean-square noise. In other embodiments, a typical noise value or a statistical value is chosen. Often, the noise is expected to follow a Poisson distribution.

As the interrogation space is translated through the sample, the laser beam directed to the interrogation space generates a burst of photons when a label is encountered. The photons emitted by the label are discriminated from background light or background noise emission by considering only the bursts of photons with energy above a predetermined threshold energy level, thereby accounting for the amount of background noise present in the sample. Background noise typically comprises low frequency emission produced, e.g., by the intrinsic fluorescence of non-labeled particles that are present in the sample, the buffer or diluent used in preparing the sample for analysis, Raman scattering and electronic noise. In some embodiments, the value assigned to the background noise is calculated as the average background signal noise detected in a plurality of bins, which are measurements of photon signals that are detected in an interrogation space during a predetermined length of time. In some embodiments, background noise is calculated for each sample as a number specific to that sample.

Given the value for the background noise, a threshold energy level can be assigned. As discussed above, the threshold value is determined to discriminate true signals resulting from the fluorescence of a label from the background noise. A threshold value can be chosen such that the number of false positive signals from random noise is minimized while the number of true signals which are rejected is also minimized. Methods for choosing a threshold value include determining a fixed value above the noise level and calculating a threshold value based on the distribution of the noise signal. In one embodiment, the threshold is set at a fixed number of standard deviations above the background level. Assuming a Poisson distribution of the noise, using this method one can estimate the number of false positive signals over the time course of the experiment. In some embodiments, the threshold level is calculated as a value of four standard deviations (σ) above the background noise. For example, given an average background noise level of 200 photons, the analyzer system establishes a threshold level of 4√200 above the average background/noise level of 200 photons to be 256 photons. Thus, in some embodiments, determining the concentration of a label in a sample includes establishing the threshold level above which photon signals represent the presence of a label. Conversely, the absence of photon signals with an energy level greater than the threshold level indicate the absence of a label.

Many bin measurements are taken to determine the concentration of a sample, and the absence or presence of a label is ascertained for each bin measurement. Typically, 60,000 measurements or more can be made in 1 min. 60,000 measurements are made in 1 min when the bin size is 1 ms. For smaller bin sizes the number of measurements is correspondingly larger, e.g., 6,000,000 measurements per minute equates to a bin size of 10 microseconds. Because so many measurements are taken, no single measurement is crucial, thus providing for a high margin of error. Bins that are determined not to contain a label (“no” bins) are discounted and only the measurements made in the bins that are determined to contain label (“yes” bins) are accounted in determining the concentration of the label in the processing sample. Discounting measurements made in the “no” bins or bins that are devoid of label increases the signal to noise ratio and the accuracy of the measurements. Thus, in some embodiments, determining the concentration of a label in a sample comprises detecting the bin measurements that reflect the presence of a label.

The signal to noise ratio or the sensitivity of the analyzer system can be increased by minimizing the time that background noise is detected during a bin measurement in which a particle-label complex is detected. For example, consider a bin measurement lasting 1 millisecond during which one particle-label complex is detected as it passes across an interrogation space in 250 microseconds. Under these conditions, 750 microseconds of the 1 millisecond are spent detecting background noise emission. The signal to noise ratio can be improved by decreasing the bin time. In some embodiments, the bin time is 1 millisecond. In other embodiments, the bin time is 750 microseconds, 500 microseconds, 250 microseconds, 100 microseconds, 50 microseconds, 25 microseconds or 10 microseconds. Other bin times are as described herein.

Other factors that affect measurements are the brightness or dimness of the fluorescent moiety, size of the aperture image or lateral extent of the laser beam, the rate at which the interrogation space is translated through the sample, and the power of the laser. Various combinations of the relevant factors that allow for detection of label will be apparent to those of skill in the art. In some embodiments, the bin time is adjusted without changing the scan speed. It will be appreciated by those of skill in the art that as bin time decreases, laser power output directed at the interrogation space must increase to maintain a constant total energy applied to the interrogation space during the bin time. For example, if bin time is decreased from 1000 microseconds to 250 microseconds, as a first approximation, laser power output must be increased approximately four-fold. These settings allow for the detection of the same number of photons in a 250 microseconds as the number of photons counted during the 1000 microseconds given the previous settings, and allow for faster analysis of sample with lower backgrounds and greater sensitivity. In addition, the speed at which the interrogation space is translated through the sample can be adjusted in order to speed processing of sample. These numbers are merely exemplary, and the skilled practitioner can adjust the parameters as necessary to achieve the desired result.

In some embodiments, the interrogation space is smaller than the volume of sample when, for example, the interrogation space is defined by the size of the spot illuminated by the laser beam. In some embodiments, the interrogation space can be defined by adjusting the apertures 182 (FIGS. 1A & 1B) of the analyzer and reducing the illuminated volume that is imaged by the objective lens to the detector. In embodiments wherein the interrogation space is defined to be smaller than the cross-sectional area of the sample, the concentration of the label can be determined by interpolation of the signal emitted by the complex from a standard curve that is generated using one or more samples of known standard concentrations. In other embodiments, the concentration of the label can be determined by comparing the measured particles to an internal label standard. In embodiments wherein a diluted sample is analyzed, the dilution factor is accounted for when calculating the concentration of the molecule of interest in the starting sample.

To determine the concentration of labels in the processing sample, the total number of labels contained in the “yes” bins is determined relative to the sample volume represented by the total number of bins. Thus, in one embodiment, determining the concentration of a label in a processing sample comprises determining the total number of labels detected “yes” and relating the total number of detected labels to the total sample volume that was analyzed. The total sample volume that is analyzed is the sample volume through which the interrogation space is translated in a specified time interval. Alternatively, the concentration of the label complex in a sample is determined by interpolation of the signal emitted by the label in a number of bins from a standard curve that is generated by determining the signal emitted by labels in the same number of bins by standard samples containing known concentrations of the label.

In some embodiments, the number of individual labels detected in a bin is related to the relative concentration of the particle in the processing sample. At relatively low concentrations, e.g., at concentrations below about 10⁻¹⁶ M, the number of labels is proportional to the photon signal detected in a bin. Thus, at low concentrations of label the photon signal is provided as a digital signal. At relatively higher concentrations, for example at concentrations greater than about 10⁻¹⁶ M, the proportionality of photon signal to a label is lost as the likelihood of two or more labels crossing the interrogation space at about the same time and being counted as one becomes significant. Thus, in some embodiments, individual particles in a sample of a concentration greater than about 10⁻¹⁶ M are resolved by decreasing the length of time of the bin measurement.

In other embodiments, the total photon signal that is emitted by a plurality of particles that are present in any one bin is detected. These embodiments allow for single molecule detectors of the disclosure wherein the dynamic range is at least 3, 3.5, 4, 4.5, 5.5, 6, 6.5, 7, 7.5, 8, or more than 8 logs.

“Dynamic range,” as that term is used herein, refers to the range of sample concentrations that can be quantitated by the instrument without need for dilution or other treatment to alter the concentration of successive samples of differing concentrations, where concentrations are determined with accuracy appropriate for the intended use. For example, if a microtiter plate contains a sample of 1 femtomolar concentration for an analyte of interest in one well, a sample of 10,000 femtomolar concentration for an analyte of interest in another well, and a sample of 100 femtomolar concentration for the analyte in a third well, an instrument with a dynamic range of at least 4 logs and a lower limit of quantitation of 1 femtomolar can accurately quantitate the concentration of all the samples without further treatment to adjust concentration, e.g., dilution. Accuracy can be determined by standard methods, e.g., measuring a series of standards with concentrations spanning the dynamic range and constructing a standard curve. Standard measures of fit of the resulting standard curve can be used as a measure of accuracy, e.g., an r² greater than about 0.7, 0.75, 0.8, 0.85, 0.9, 0.91, 0.92, 0.93, 0.94, 0.95, 0.96, 0.97, 0.98, or 0.99.

Dynamic range can be increased by altering how data from the detector is analyzed, and perhaps using an attenuator between the detector and the interrogation space. At the low end of the range, the processing sample is sufficiently dilute that each detection event, i.e., each burst of photons above a threshold level in a bin (the “event photons”), likely represents only one label. Under these conditions, the data is analyzed to count detection events as single molecules so that each bin is analyzed as a simple “yes” or “no” for the presence of label, as described above. For a more concentrated processing sample, where the likelihood of two or more labels occupying a single bin becomes significant, the number of event photons in a significant number of bins is substantially greater than the number expected for a single label. For example, the number of event photons in a significant number of bins corresponds to two-fold, three-fold, or more than the number of event photons expected for a single label. For these samples, the instrument changes its method of data analysis to integrate the total number of event photons for the bins of the processing sample. This total is proportional to the total number of labels in all the bins. For an even more concentrated processing sample, where many labels are present in most bins, background noise becomes an insignificant portion of the total signal from each bin, and the instrument changes its method of data analysis to count total photons per bin (including background). An even further increase in dynamic range can be achieved by the use of an attenuator between the sample plate and the detector, when concentrations are such that the intensity of light reaching the detector would otherwise exceed the capacity of the detector for accurately counting photons, i.e., saturate the detector.

The instrument can include a data analysis system that receives input from the detector and determines the appropriate analysis method for the sample being run, and outputs values based on such analysis. The data analysis system can further output instructions to use or not use an attenuator, if an attenuator is included in the instrument. For instance, the data processing system includes a processor operatively connected to the detector, wherein the processor is configured to execute instructions stored on a non-transitory computer-readable medium, and wherein the instructions, when executed by the processor, cause the processor to operate in anyone of the following manners: determine a threshold photon value corresponding to a background signal in the interrogation space, determine the presence of a photon emitting moiety in the interrogation space in each of a plurality of bins by identifying bins having a photon value greater than the threshold value, and compare the number of bins having a photon value greater than the threshold value to a standard curve.

By utilizing such methods, the dynamic range of the instrument can be dramatically increased. In some embodiments, the instrument is capable of measuring concentrations of samples over a dynamic range of more than about 1000 (3 log), 10,000 (4 log), 100,000 (5 log), 350,000 (5.5 log), 1,000,000 (6 log), 3,500,000 (6.5 log), 10,000,000 (7 log), 35,000,000 (7.5 log), or 100,000,000 (8 log). In some embodiments, the instrument is capable of measuring the concentrations of samples over a dynamic range of from about 1 to 10 femtomolar to at least about 1000, 10,000, 100,000, 350,000, 1,000,000, 3,500,000, 10,000,000, or 35,000,000 femtomolar.

In some embodiments, an analyzer or analyzer system of the disclosure is capable of detecting an analyte, e.g., a biomarker, at a limit of detection of less than about 1 nanomolar, or 1 picomolar, or 1 femtomolar, or 1 attomolar, or 1 zeptomolar. In some embodiments, the analyzer or analyzer system is capable of detecting a change in concentration of the analyte, or of multiple analytes, e.g., a biomarker or biomarkers, from one sample to another sample of less than about 0.1%, 1%, 2%, 5%, 10%, 20%, 30%, 40%, 50%, 60%, or 80% when the biomarker is present at a concentration of less than about 1 nanomolar, or 1 picomolar, or 1 femtomolar, or 1 attomolar, or 1 zeptomolar, in the samples, and when the size of each of the sample is less than about 100, 50, 40, 30, 20, 10, 5, 2, 1, 0.1, 0.01, 0.001, or 0.0001 μl.

Methods of Use of Single Molecule Analyzer

Further provided herein is a method for detecting the presence or absence of a single molecule in a sample comprising: (a) directing electromagnetic radiation from an electromagnetic radiation source to an interrogation space in the sample; (b) detecting the presence or absence of a first single molecule in the interrogation space located at a first position in the sample; (c) translating the interrogation space through the sample to a subsequent position in the sample; (d) detecting the presence or absence of a subsequent single molecule in the subsequent position in the sample; and (e) repeating steps (c) and (d) as required to detect the presence or absence of a single molecule in more than one position of the sample

Further provided herein is a method for detecting the presence or absence of a single molecule wherein the interrogation space is translated in a non-linear path. In a further embodiment, the non-linear path comprises a substantially circular path. In another embodiment, the non-linear path comprises a helical pattern. The disclosure provides for a method of detecting the presence or absence of a single molecule in an interrogation space wherein the interrogation space is translated through the sample. In some embodiments, the method provides for the sample to remain substantially stationary relative to the instrumentation. In some embodiments, the method provides that the sample is translated with respect to the instrumentation. In some embodiments, both the sample and the electromagnetic radiation are translated with respect to one another. For instance, the sample container can be moved in a linear pattern to minimize movement of the container while the electromagnetic radiation is moved in a non-linear pattern or a linear pattern that bisects but does substantially overlap the linear pattern of movement of the sample. In an embodiment where the sample is translated with respect to the instrumentation, the sample can remain stationary within its container, e.g., a microwell. While single molecules can diffuse in and out of an interrogation space or a series of interrogations spaces, the medium in which the single molecules are present remains stationary. Therefore, this system allows for single molecule detection without the need for flowing fluid.

Multiplexed Single Molecule Analyzer

As noted above, the disclosure also provides for multiplexed systems and methods according to additional aspects. In general, a multiplexed single molecule analyzer system can include one or more electromagnetic radiation sources, a system for directing electromagnetic radiation from such source(s) to one or more interrogation spaces in a sample, a translating system, and one or more detectors. Additionally, for example, the multiplexed analyzer system can include a processor for processing emitted electromagnetic radiation (e.g., photons) detected by the detector(s). Thus, in some respects, the multiplexed systems and methods of the disclosure may be similar to the singleplex systems and methods described above.

In multiplexed systems and methods, however, the analyzer system can detect and analyze multiple, different types of target molecules in a single reaction well. To do so, the multiplexed analyzer system can distinguish one type of target molecule from the others. This can be achieved, in part, by labeling the different target molecules with different labels, which have (i) excitation wavelength bands that differ from one another and/or (ii) emission wavelength bands that differ from one another. In some implementations, the different labels have excitation wavelength bands and/or emission wavelength bands with relatively little overlap or no overlap. In additional or alternative implementations, there may be some overlap among the excitation wavelength bands and/or the emission wavelength bands of the labels.

In implementations in which the excitation wavelength bands of the labels differ from one another, the multiplexed analyzer system can include one or more electromagnetic radiation sources that can provide excitation radiation within the different excitation wavelength bands of those labels. The system can thus controllably provide electromagnetic radiation at different wavelengths to selectively excite particular label(s) to detect a particular one of the target molecules.

In one example, the system can include multiple electromagnetic radiation sources that each provides electromagnetic radiation at a different wavelength. In an additional or alternative example, a given one of the electromagnetic radiation source(s) can use optical filters to provide electromagnetic radiation at different wavelengths. For instance, the multiplexed analyzer system can be configured to selectively switch between multiple optical filters to thereby selectively provide electromagnetic radiation at different wavelengths to an interrogation space. Alternatively, for instance, the system can simultaneously provide electromagnetic radiation at different wavelengths from a given one of the electromagnetic radiation source(s) by splitting and separately filtering electromagnetic radiation provided by the source. Other examples may also be possible.

In implementations in which the emissions wavelength bands of the labels differ from one another, the multiplexed analyzer system can include one or more detectors that can detect emission radiation within the different emission wavelength bands of those labels. In one example, the multiplexed analyzer system can include multiple detectors that each detects emission radiation at a different wavelength. For instance, the multiplexed system can include an optical filter in the optical path of each detector to help ensure that the detector receives photons from only a corresponding label type. In an additional or alternative example, the multiplexed analyzer system can selectively change between different optical filters in the optical path of a given one of the detector(s) to allow that detector to detect emission radiation at different wavelengths. Other examples may also be possible.

More generally, to perform a multiplex assay on a sample in a single well, the multiplexed analyzer system can include one or more electromagnetic radiation sources and/or one or more detectors to excite and detect different types of label labeling target molecules in the sample. Although numerous examples are possible, the following examples are described below: (1) a system having a single electromagnetic radiation source and multiple detectors; (2) a system having multiple electromagnetic radiation sources and a single detector; and (3) a system having multiple electromagnetic radiation sources and multiple detectors.

By providing excitation radiation and detecting emission radiation at multiple wavelengths within a single reaction well, reagent costs, the required sample volume, and the time required to get results for multiple target molecules can be reduced.

Single Electromagnetic Radiation Source and Multiple Detectors

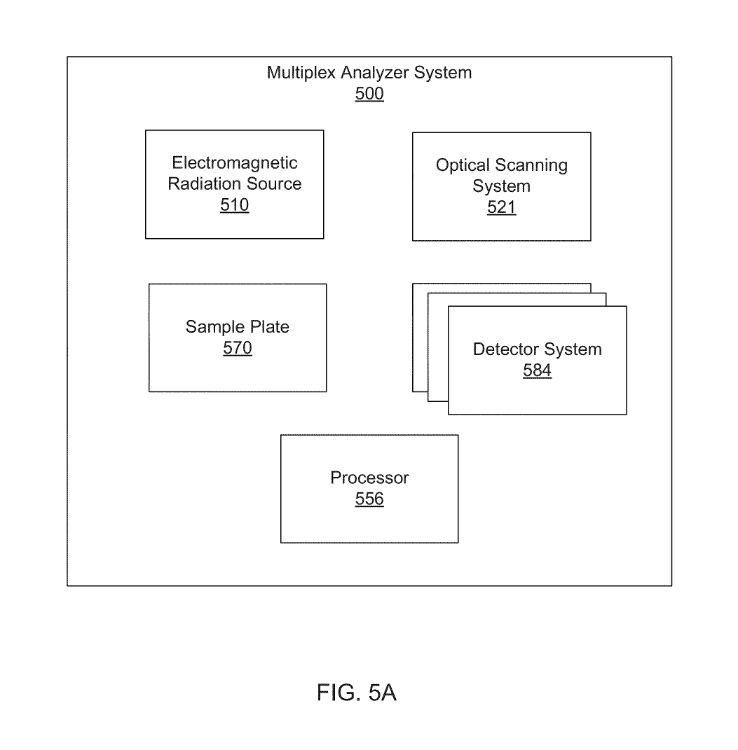

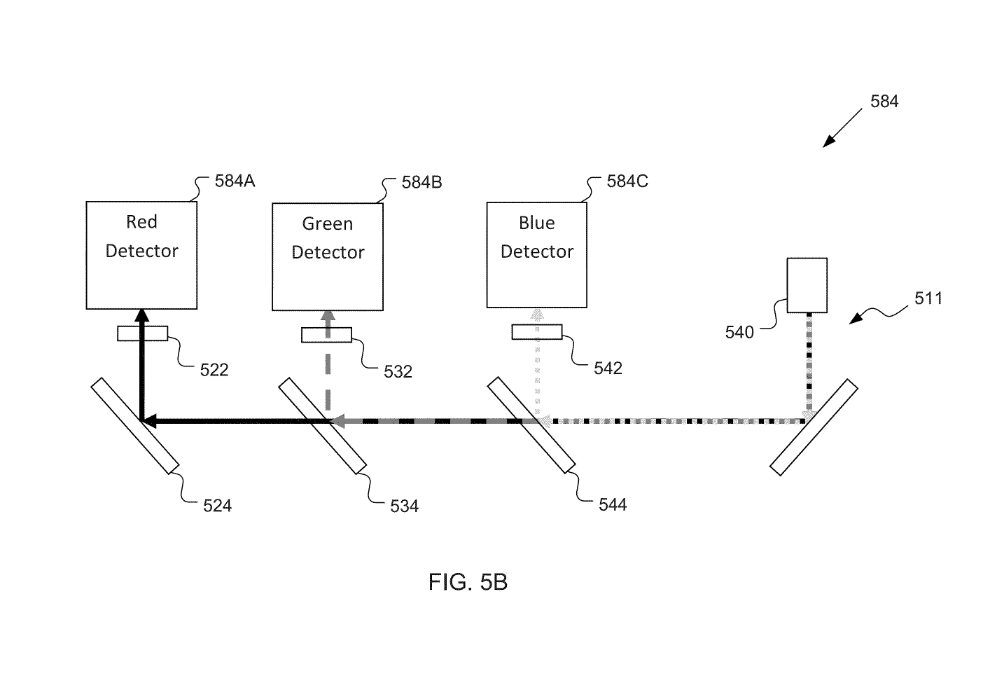

FIG. 5A shows a simplified schematic diagram of a multiplexed analyzer system 500 according to one example. As shown in FIG. 5A, the example system 500 includes a single electromagnetic radiation source 510 and a detector system 584, which includes multiple detectors. As also shown in FIG. 5A, the example system 500 further includes an optical scanning system 521, a sample plate 570, and a processor 556. These components of the system 500 can be the same as or similar to the corresponding components described above with respect to FIGS. 1A and 1B. The system 500 can additionally or alternatively include any other component described above with respect to FIGS. 1A and 1B (e.g., one or more alignment mirrors, dichroic mirrors, scan mirrors, objective lenses, etc.).

The multiplexed analyzer system 500 can thus direct electromagnetic radiation from the electromagnetic radiation source 510 to an interrogation space in the sample on the sample plate 570. Additionally, the multiplexed analyzer system 500 can translate the interrogation space through at least a portion of the sample, thereby forming a moveable interrogation space. For example, as described above, the optical scanning system 521 can include a scan motor and mirror coupled to a motor to translate the interrogation space through the sample. In some implementations, the interrogation space is translated through the sample using multiple mirrors or a prism coupled to the scan motor.

As noted above, to perform a multiplex assay, the multiplexed analyzer system 500 can utilize a different label for each of multiple target molecules. The single electromagnetic radiation source 510 can provide electromagnetic radiation to a sample at wavelengths within an excitation wavelength band of each label that is utilized for labeling target molecules. For example, in an implementation in which the system 500 employs at least a first label for labeling a first target molecule and a second label for labeling a second target molecule, the electromagnetic radiation source 510 can provide electromagnetic radiation in at least an excitation wavelength band of the first label and an excitation wavelength band of the second label. In additional or alternative examples, the labels can include two or more labels, three or more labels, four or more labels, five or more labels, six or more labels, seven or more labels, etc.

The detector system 584 includes multiple detectors that each detects electromagnetic radiation emitted from a respective label located in the interrogation space. For instance, in the implementation in which the system 500 employs at least the first label and the second label, the detector system 584 can include at least a first detector and a second detector configured to detect electromagnetic radiation emitted by the first label and the second label, respectively, if present in the interrogation space.

In some implementations, the multiplexed analyzer system 500 can utilize labels having excitation wavelength bands that do not overlap. In some of such implementations, the band of wavelengths provided by the source of electromagnetic radiation can be broad enough to encompass the excitation bands of all labels. Additionally or alternatively, the system 500 can include one or more filters, switches, or other optics to limit the wavelengths of electromagnetic radiation reaching the interrogation space such that only one of the label species is excited at a time in an alternating fashion. Such a configuration would also allow for the use of a single detector.

In additional or alternative implementations, the multiplexed analyzer system 500 can utilize labels in which an excitation wavelength band of one label overlaps with an excitation wavelength band of at least one other label. In some of such implementations, it may be beneficial to reduce or limit the amount of overlap of the label emission bands to prevent false positives due to a label of one species being detected by a non-corresponding detector.

The processor 556 can be operatively coupled to the detector system 584 for determining the target molecules in a manner similar to that described above in Section V(C). For example, the processor 556 can receive from each of the detectors photon count signals. The photon count signals can indicate a photon count value in each of a plurality of bins for a respective one of the labels detected by that detector in the interrogation space. The processor 556 can determine, based on the photon count signals, each bin in the plurality of bins having a photon value that is greater than a respective threshold value. For each bin having a photon value greater than the respective threshold, the processor 556 determines that a label, corresponding to one of the target molecules, was present in the interrogation space. The threshold value can relate to a background signal in the interrogation space at an emission wavelength of the corresponding label.

For instance, in the implementation in which the system 500 employs at least the first label and the second label, the processor 556 receives photon count signals (from the detectors) indicating a photon count value for the first label detected in the interrogation space in each bin of a first plurality of bins and a photon count value for the second label detected in the interrogation space in each bin of a second plurality of bins. The processor 556 is further configured to identify each bin of the first plurality of bins having a photon value for the first label greater than a first threshold value and identify each bin of the second plurality of bins having a photon value for the second label greater than a second threshold value. For each bin having a photon value greater than the respective threshold, the processor 556 determines that a label, corresponding to a single target molecule, was present in the interrogation space. The first and second threshold photon values correspond to a background signal in the interrogation space at an emission wavelength of the first label and at a background signal in the interrogation space at an emission wavelength of the second label, respectively.

Additionally, for example, the processor 556 can determine a concentration of each target molecule as a function of a sum of the number of bins having a photon value for the label corresponding to that target molecule that is greater than the respective threshold value for the detector detecting that label. For instance, in the implementation in which the system 500 includes at least a first label and a second label, the processor 556 can determine (i) the concentration of the first target molecule as a function of a sum of the number of bins having a photon value for the first label that is greater than a first threshold value, and (ii) the concentration of the second target molecule as a function of a sum of the number of bins having a photon value for the second label that is greater than a second threshold value. The first threshold level may be the same as the second threshold level.

As described above, the multiplexed analyzer system 500 can excite the different labels at the same time or in an alternating manner, and responsively detect the emission radiation from the labels at the same time or in an alternating manner. Where emissions from the labels are detected at the same time, the plurality of bins for each label can be the same bins (e.g., the first plurality of bins and the second the plurality of bins within which photon count values are determined may comprise the same bins). Where emissions from the labels are detected sequentially (i.e., in an alternating manner), the plurality of bins can be different for each label (e.g., the first plurality of bins may be different than the second plurality of bins). The bin time for each of the plurality of bins may be the same, for example, approximately 100 μs.

In additional or alternative aspects, in configurations where the multiplexed analyzer system 500 includes a single source of electromagnetic radiation 510 and multiple detectors 584, the processor 556 can multiplex the labels to discriminate between a greater number of target molecules than there are detectors 578. To do so, one or more target molecules can be associated with combinations of two or more labels.

For example, if the system 500 includes two separate detectors configured to detect the emission wavelengths of two different labels, A and B, then it is possible to detect three different combination of labels and, thus, determine three different target molecule species—A, B, and AB. In this example, the processor 556 determines threshold photon values corresponding to a background signal in the interrogation space at an emission wavelength of each of the first and second labels. The processor 556 determines the first label corresponding to the first target molecule in the interrogation space by identifying each bin of a first plurality of bins having a photon value for the first label greater than a first threshold value. Similarly, the processor 556 determines the second label corresponding to the second target molecule in the interrogation space by identifying each bin of a second plurality of bins having a photon value for the second label greater than a second threshold value. The processor 556 determines a combination of the first label and the second label corresponding to the third target molecule in the interrogation space by identifying each bin of a third plurality of bins having a photon value for each of the first label and the second label greater than the first and the second threshold values. Similar to that described above, the processor 556 can determine a concentration of the first target molecule and second target molecule as a function of a sum of the number of bins having a photon value for the first label and the second label that is greater than the respective threshold value. The processor 556 determines a concentration of the third target molecule as a function of a sum of the number of bins having a photon values for both of the first label and the second label that are greater than the first and the second threshold values.

In another example system 500, three detectors configured to detect electromagnetic radiation from three label species—A, B and C—may be used to detect up to seven different target molecule species—A, B, C, AB, AC, BC, ABC—with a single analyzer. In some cases, the ability of the processor 556 to distinguish between species associated with a set of labels and species associated with individual labels may depend on the density of labeled molecules in the sample container being low enough such that an A species and a B or C species will not be present in the interrogation space at the same time by coincidence. Alternatively, if the counts for each of the species are high, mathematical deconvolution may be used to determine photon counts for each of the species, even when multiple label species or combination label species are present in the interrogation space at the same time.

FIG. 5B illustrates one example of the detector system 584 (of FIG. 5A) having three detectors 584A-584C. In this embodiment, up to three different labels may be used alone, or as described above, in combination. Detector system 584, may include a transmission system 511 for directing electromagnetic radiation emitted from the interrogation space and received at an objective lens 540. In the illustrated example, the detector system 584 includes a red detector 584A with an associated emission filter 522 and detector dichroic filter 524; a green detector 584B with an associated emission filter 532 and detector dichroic filter 534; and a blue detector 584C with an associated emission filter 542 and detector dichroic filter 544. Each of the dichroic filters 524, 534, 544 can help to ensure that its respective detector 584A-584C receives photons from a corresponding label type.

In some implementations, the objective lens 540 can include an optical coating that allows for transmission of photons emitted from the labels to the detectors 584A-584C, but blocks transmission of at least a portion of radiation outside the label's emission wavelength bands. In one example, the optical coating can allow for transmission of photons having a wavelength in a range of approximately 400 nm to approximately 700 nm and, in another example, in a range of approximately 421 nm to approximately 647 nm. Additionally, the optical coating can allow for transmission of electromagnetic radiation from the electromagnetic energy source 510 to the interrogation space. For example the optical coating can allow for transmission of excitation radiation in a range from approximately 350 nm to approximately 700 nm and, in another example, in a range from approximately 405 nm to approximately 635 nm.

The example optical arrangement illustrated in FIG. 5B may be used in any analyzer system described herein utilizing multiple detectors. Using a single electromagnetic radiation source may beneficially reduce costs of the multiplexed analyzer system relative to other example systems including multiple electromagnetic radiation sources.

Multiple Electromagnetic Radiation Sources and a Single Detector

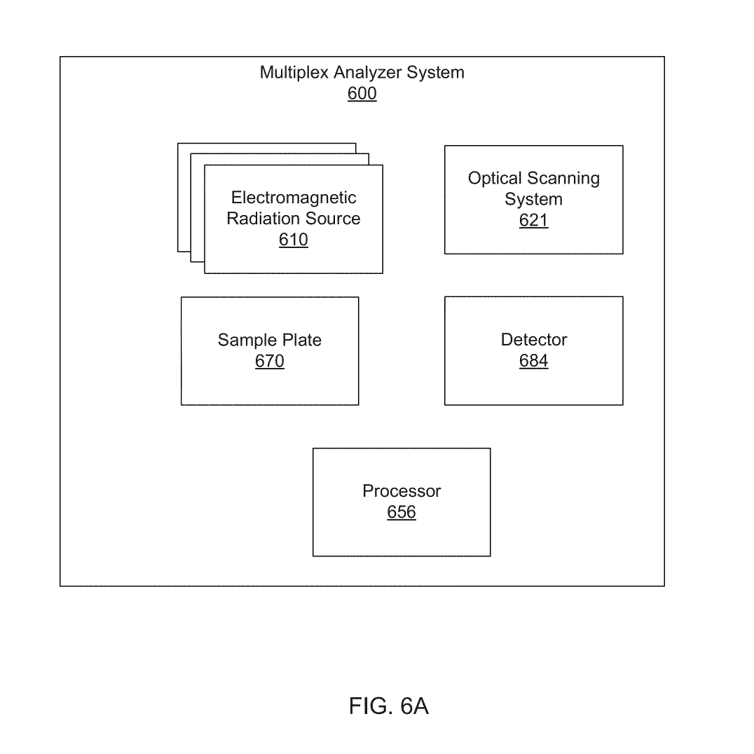

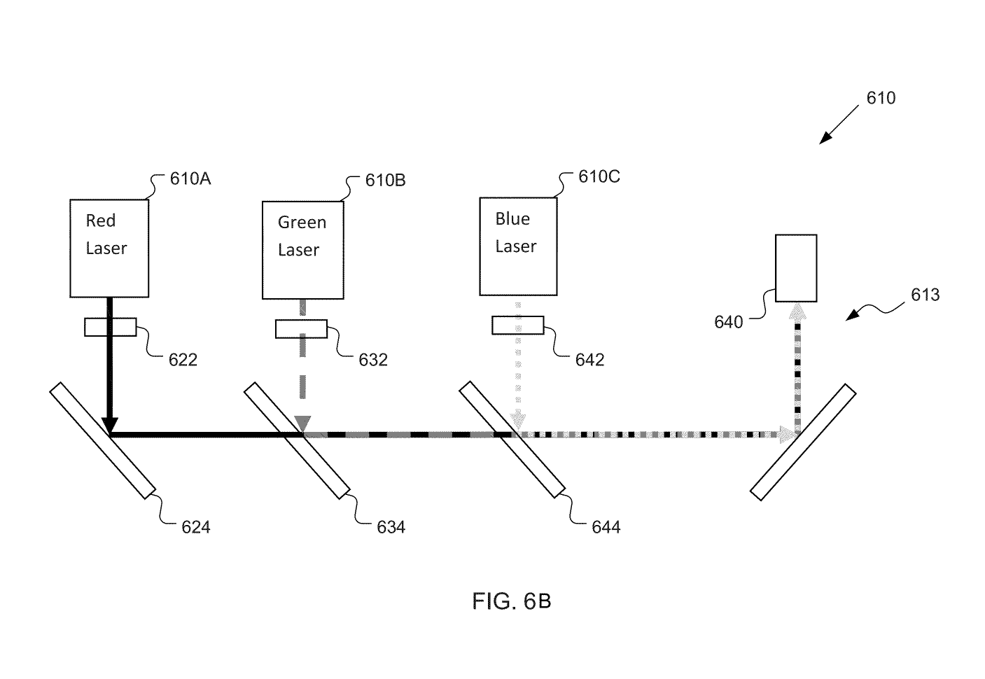

FIG. 6A shows a simplified schematic diagram of a multiplexed analyzer system 600 according to another example. As shown in FIG. 6A, the example system 600 includes an electromagnetic radiation source system 610, comprising multiple electromagnetic radiation sources, and a single detector 684. Also, as shown in FIG. 6A, the example system 600 further includes an optical scanning system 621, a sample plate 670, and a processor 656. These components of the system 600 can be the same as or similar to the corresponding components described above with respect to FIGS. 1A and 1B. The system 600 can additionally or alternatively include any other component described above with respect to FIGS. 1A and 1B (e.g., one or more alignment mirrors, dichroic mirrors, scan mirrors, objective lenses, etc.).

The multiplexed analyzer system 600 can direct electromagnetic radiation from the electromagnetic radiation sources 610 to one or more interrogation spaces in the sample on the sample plate 670. Additionally, the multiplexed analyzer system 600 can translate the interrogation space(s) through at least a portion of the sample, thereby forming moveable interrogation space(s). For instance, as described above, the optical scanning system 621 can include one or more scan motors and mirrors coupled to one or more motors to translate the interrogation space(s) through the sample. In some examples, the electromagnetic radiation emitted from each of the electromagnetic radiation sources 610 can be directed to a single interrogation space by focusing the emitted electromagnetic radiation on a single detection spot. By focusing the several sources 610 of electromagnetic radiation onto a single detection spot, emission radiation from the labels in the single interrogation space can be detected with a single detector and, optionally, the multiplexing of combinations of labels as described above can be achieved.

In one aspect, each electromagnetic radiation source can provide electromagnetic radiation to the interrogation space(s) at a wavelength within an excitation wavelength band of a respective one of the multiple labels used to label the target molecules of interest. For example, a first electromagnetic radiation source can provide electromagnetic radiation at a first excitation wavelength, which is within an excitation wavelength band of a first label, and a second electromagnetic radiation source can provide electromagnetic radiation at a second excitation wavelength, which is within an excitation wavelength band of a second label. The first label can be used to label a first target molecule and the second label can be used to label a second target molecule.

In some implementations, each electromagnetic radiation source can provide electromagnetic radiation at a wavelength that overlaps with the excitation wavelength band of the label to which that electromagnetic radiation source corresponds, but have relatively little or no overlap with excitation wavelength bands of any other label. For example, a first electromagnetic radiation source can provide electromagnetic radiation at a wavelength that overlaps with an excitation wavelength band of a first label but not an excitation wavelength band of a second label, and a second electromagnetic radiation source can provide electromagnetic radiation at a wavelength that overlaps with the excitation wavelength band of the second label but not the excitation wavelength band of the first label.

In additional or alternative implementations, the excitation wavelength bands of the labels have relatively little or no overlap with one another, and/or the electromagnetic radiation sources can provide electromagnetic radiation at respective wavelengths that have relatively little or no overlap with one another.

If a label in the interrogation space(s) is excited by its corresponding electromagnetic radiation source 610, the detector 684 detects electromagnetic radiation emitted by the label. In some examples, to facilitate determining which label emitted radiation from the interrogation space using the single detector 684, the multiplexed analyzer system 600 can cause the electromagnetic radiation sources 610 to provide electromagnetic radiation to the interrogation space(s) one at a time (e.g., in a sequential or alternating manner). A single detector system may therefore reduce or eliminate the potential for cross-talk between detection channels.

The processor 656 can be operatively coupled to the detector 684 for determining the target molecules in a manner similar to that described above. For example, the processor 656 can receive from the detector 684 photon count signals indicating a photon count value, in each of a plurality of bins, for a respective one of the labels detected by that detector in the interrogation space. The processor 656 can determine, based on the photon count signals, each bin in the plurality of bins having a photon value that is greater than a threshold value. For each bin having a photon value greater than the respective threshold, the processor 656 determines that a label, corresponding to one of the target molecules, was present in the interrogation space. The threshold value can relate to a background signal in the interrogation space at an emission wavelength of the corresponding label.

For instance, in an implementation in which the system 600 employs at least the first label labeling a first target molecule and the second label labeling a second target molecule, the processor 656 can detect the first target molecule when a first electromagnetic radiation source provides electromagnetic radiation to the interrogation space(s) and the processor 656 can detect the second target molecule when a second electromagnetic radiation source provides electromagnetic radiation to the interrogation space(s). In particular, the processor 656 can determine the presence of the first label corresponding to the first target molecule in the interrogation space(s) by identifying in each bin of a first plurality of bins having a photon value for the first label greater than a first threshold value. Similarly, the processor 656 can determine the presence of the second label corresponding to the second target molecule in the interrogation space(s) by identifying in each bin of a second plurality of bins having a photon value for the second label greater than a second threshold value. For each bin having a photon value greater than the respective threshold, the processor 656 determines that a label, corresponding to a single target molecule, was present in the interrogation space.

The first threshold photon value corresponds to a background signal in the one interrogation space(s) at an emission wavelength of the first label and the second threshold photon value corresponds to a background signal in the at least one interrogation space at an emission wavelength of the second label. In some examples, the first plurality of bins may be different than the second plurality of bins.

Where a third electromagnetic radiation source for providing electromagnetic radiation at a third excitation wavelength to at least one interrogation space is provided, the processor 656 can be configured to determine the third target molecule when the third electromagnetic radiation source is emitting. Similar to that described above, the presence of the third label corresponding to the third target molecule in the interrogation space(s) is determined in each bin of a third plurality of bin times by identifying bins having a photon value for the third label greater than a third threshold value. The third threshold photon value corresponds to a background signal in the at least one interrogation space at an emission wavelength of the third label. In this embodiment, each bin of the first, second and third plurality of bins may be different bins.

FIG. 6B illustrates one example of the electromagnetic radiation source system 610 (of FIG. 6A) having three electromagnetic radiation sources for providing electromagnetic radiation to the interrogation space. In the illustrated example the sources include a red laser 610A, a green laser 610B, and a blue laser 610C. Electromagnetic radiation source system 610 may include a transmission system 613 for directing electromagnetic radiation emitted from the sources to the interrogation space via an objective 640. The objective 640 may be the same as or similar to the objective 540 described above. The system 600 may further include cleanup filters 622, 632, 642 and dichroic mirrors 624, 634, 644 associated with each respective source 610A, 610B, 610C. The optical arrangement illustrated in FIG. 6 may be used in any analyzer system described herein utilizing multiple electromagnetic sources.

For this configuration, instrument cost may be reduced as only one detector is used.

Multiple Electromagnetic Radiation Sources and Multiple Detectors

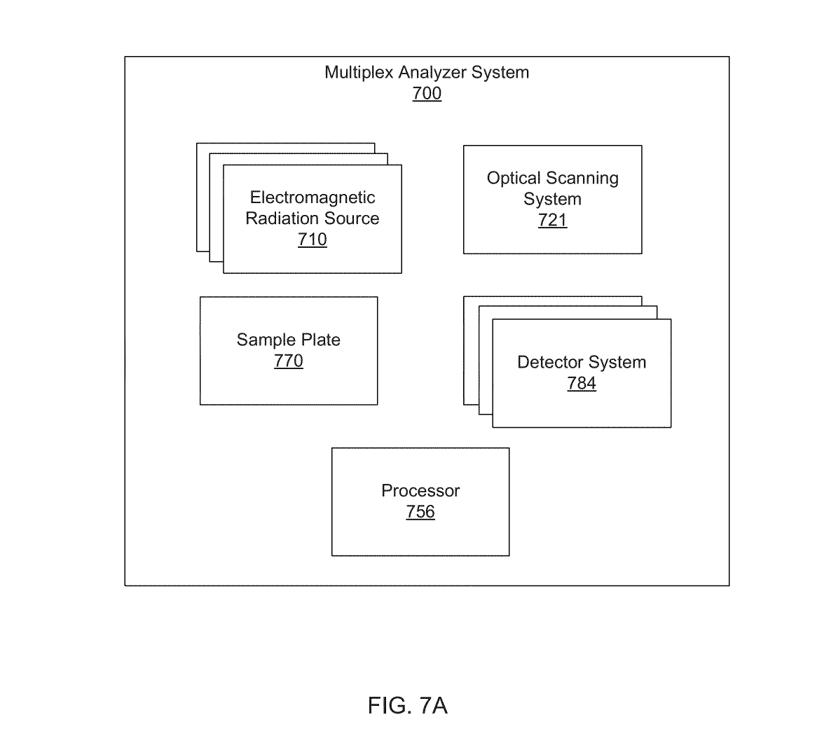

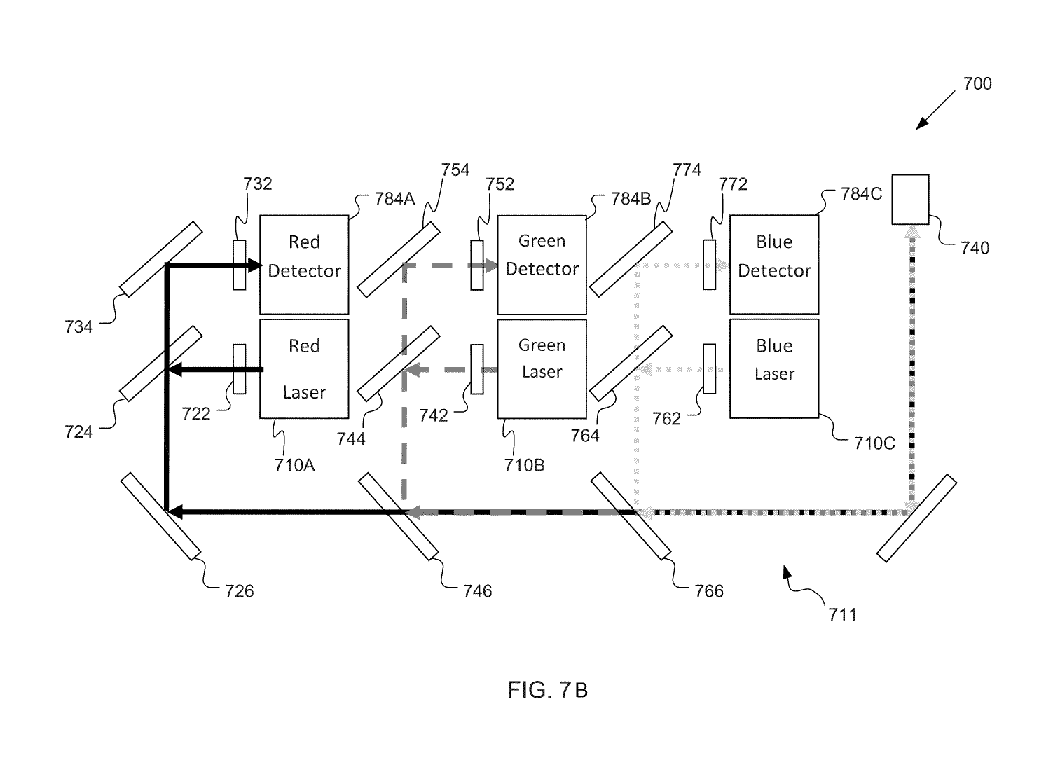

In another example, a multiplexed analyzer system can include a source of electromagnetic radiation comprising multiple sources, such as the system 600 shown in FIG. 6, and a detection system comprising multiple detectors, such as the system 500 shown in FIG. 5. FIG. 7A shows a simplified schematic diagram of a multiplexed analyzer system 700 according to such an example. As shown in FIG. 7A, the example system 700 includes an electromagnetic radiation source system, comprising multiple electromagnetic radiation sources 710, and a detector system, comprising multiple detectors 784. Also, as shown in FIG. 7A, the example system 700 further includes an optical scanning system 721, a sample plate 770, and a processor 756. These components of the system 700 can be the same as or similar to the corresponding components described above with respect to FIGS. 1A and 1B. The system 700 can additionally or alternatively include any of the other components described above with respect to FIGS. 1A and 1B (e.g., one or more alignment mirrors, dichroic mirrors, scan mirrors, objective lenses, etc.).

In one implementation, each of the multiple detectors 784 can be configured to detect electromagnetic radiation emitted by a different label species. In some examples, the detection system 700 includes two detectors 784, and in further examples, the detection system includes three detectors 784. In general, the system 700 can include two or more electromagnetic radiation sources 710 and two or more detectors 784.

The processor 756 can determine the target molecules and concentrations thereof based on photon count signals, photon count values, bins, and thresholds as described above.

FIG. 7B illustrates an example implementation of aspects of the system 700. In FIG. 7B, the system 700 includes three sources of electromagnetic radiation, for example, a red laser 710A, a green laser 710B and a blue laser 710C, and a detector system having three detectors, for example, a red detector 784A, a green detector 784B and a blue detector 784C. A system 711 may also be provided for directing electromagnetic radiation emitted from the sources 710A, 710B, 710C to an interrogation space and for directing electromagnetic radiation emitted from the interrogation space by one or more labels to the detectors 784A, 784B, 784C, via an objective 740. The system 711 may also include other optical elements including source cleanup filters 722, 742, 762 for each of the sources 710A-710C, respectively, and emission filters 732, 752, 772 for each of the detectors 784A-784C, respectively. A number of dichroic mirrors, for example, red dichroic mirrors 724, 726, 734, green dichroic mirrors 742, 746, 754, and blue dichroic mirrors 762, 766 and 774, can also be included.

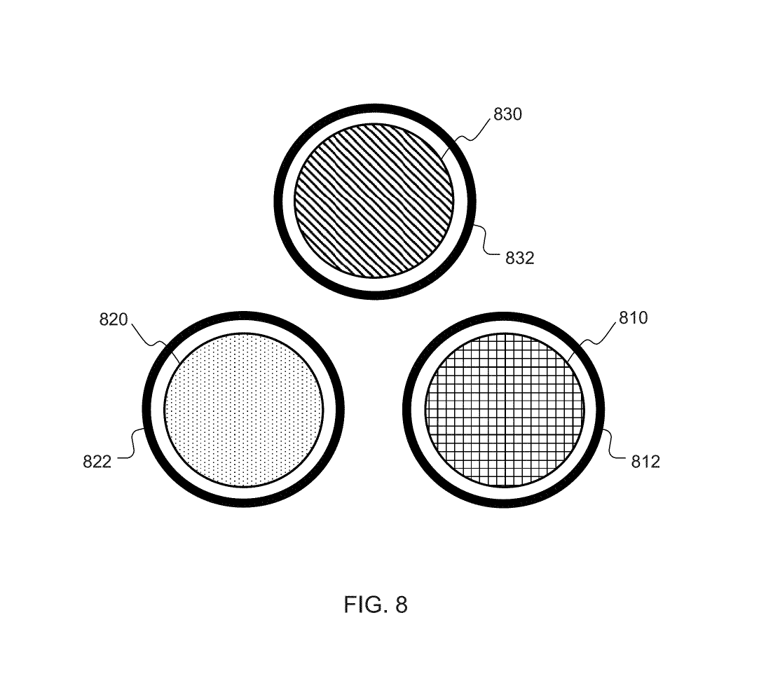

With the expense of multiple lasers and detectors, this configuration may allow for the detection of as many unique species as there are laser/detector pairs, similar to other configurations with multiple detectors. By utilizing multiple sources of electromagnetic radiation and multiple detectors, the electromagnetic radiation emitted from each of the sources may be directed to more than one interrogation space by focusing the electromagnetic radiation emitted from each source to a respective detection spot, as shown in FIG. 8.

Electromagnetic radiation emitted from each interrogation space is directed to a respective detector. For example, as shown in FIG. 8, the detection spots of each source, for example, red 820, blue 830 and green 840, may be separated and the emitted light from each spot may be directed to their respective detectors, for example, a red detector 822, blue detector 832, and green detector 842. While the interrogation spaces for each of the label species are be separated, they may remain within the view of a single objective. This separation of the detection spots can reduce cross-talk between the detection channels and may allow for the possibility of using more fluorescent labels simultaneously, even if there is some amount of overlap in the excitation and/or emission bands for the different labels.

Variations to the Multiplexed Analyzer System Examples

The multiplexed analyzer systems described and illustrated with respect to FIGS. 5A-7B provide some non-limiting examples of a multiplexed analyzer system according to aspects of the disclosure. Other examples are also possible. Additionally, the concepts described above with respect to these examples can be combined and modified in various ways according to additional or alternative aspects of the disclosure.

For instance, in one additional example, a multiplexed analyzer system can employ one or more filters, switches, or other optics in connection with an electromagnetic radiation source and/or a detector to facilitate providing and/or detecting electromagnetic radiation at more than one wavelength. This may allow for an electromagnetic radiation source and/or a detector to correspond with more than one label. In some of such examples, the excitation radiation can be provided in a sequential (and/or alternating manner) to facilitate excitation delivery and/or emission detection in a manner that can be distinguished by the multiplexed analyzer system. In one implementation of such examples, a multiplexed analyzer system can include a single electromagnetic radiation source that provides electromagnetic radiation at more than one wavelength (e.g., via filter(s), switch(es), etc.) and a single detector that detects electromagnetic radiation at more than one wavelength (e.g., via filter(s), switch(es), etc).

Additionally, although the illustrated examples may include one or three electromagnetic radiations sources and/or one or three detectors, the multiplexed analyzer systems can include one or more, two or more, three or more, four or more, five or more, etc. electromagnetic radiation sources and/or detectors in other examples.

Cross-Talk

One issue that may arise with multiplexed analyzer systems as described herein is cross-talk between labels. For example, if electromagnetic radiation emitted by label A is detected in the detector for label B, the processor may count a B type molecule where none exists. This may be mitigated or prevented by separating the electromagnetic radiation that is directed to label B from that directed to label A, either spatially or in wavelength. In one implementation, this can be achieved by using labels with narrow emission bands to reduce or prevent the possibility of overlap. However, as many labels have similar Stoke's shifts and emission bandwidths, cross-talk may be reduced or eliminated by selecting labels with separation in their emission wavelengths so that the tails of the emission band do not substantially overlap the other detector bands.

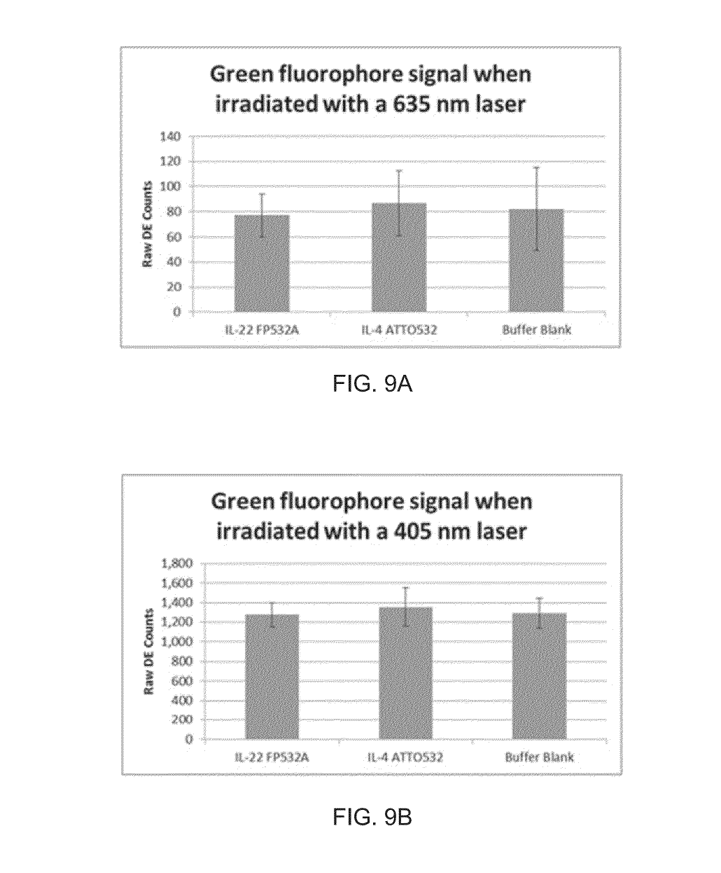

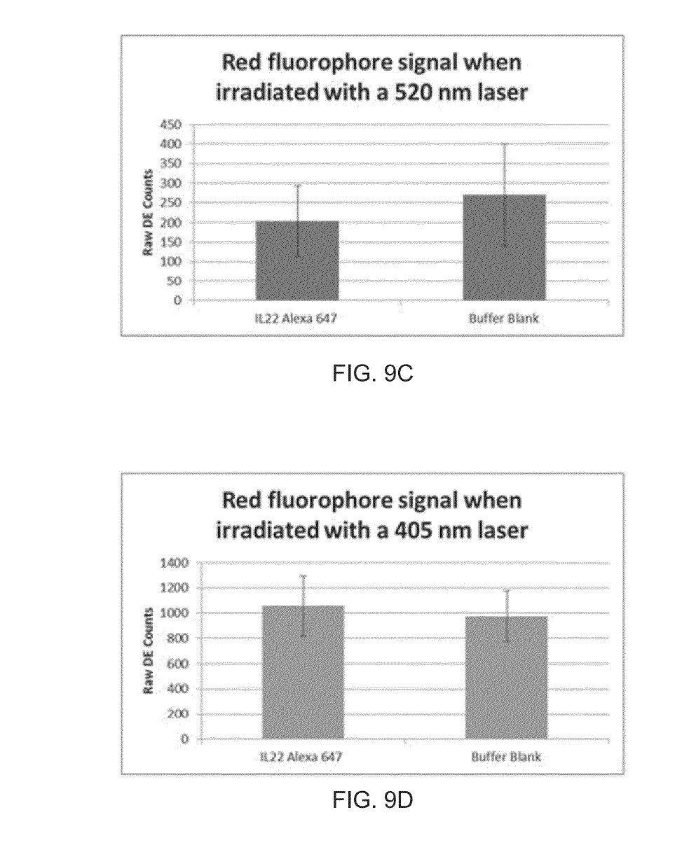

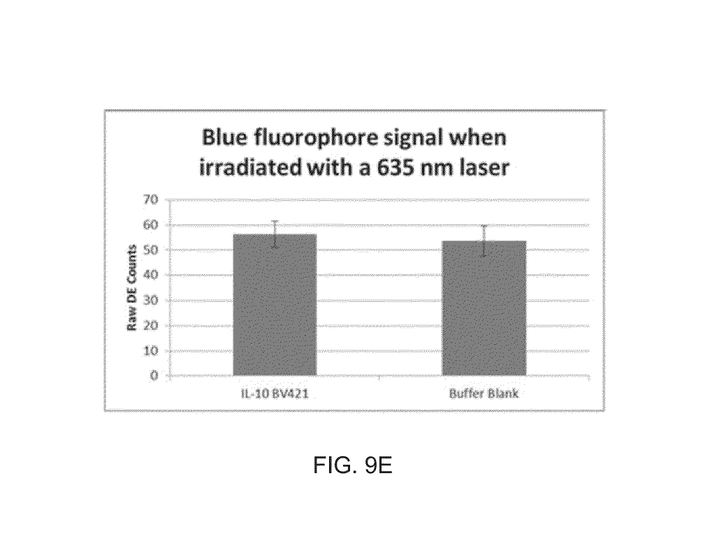

FIGS. 9A-9E show the results of experiments to determine if excitation at a wavelength removed from the maximal excitation point of a fluorophore would produce any detectable fluorescence by a multiplexed analyzer. Fluorophores with peak excitations of 405 nm, 532 nm and 647 nm were excited by each of a 405 nm laser (blue), a 520 nm laser (green) and a 635 nm laser (red). The fluorophores were associated either with target molecules IL-22 or IL-4. The received excitation signal level was compared to a buffer control to determine if there was any bleedthrough of signal. None of the combinations exhibited bleedthrough. Additional photobleaching tests and examples are described below.

Photobleaching

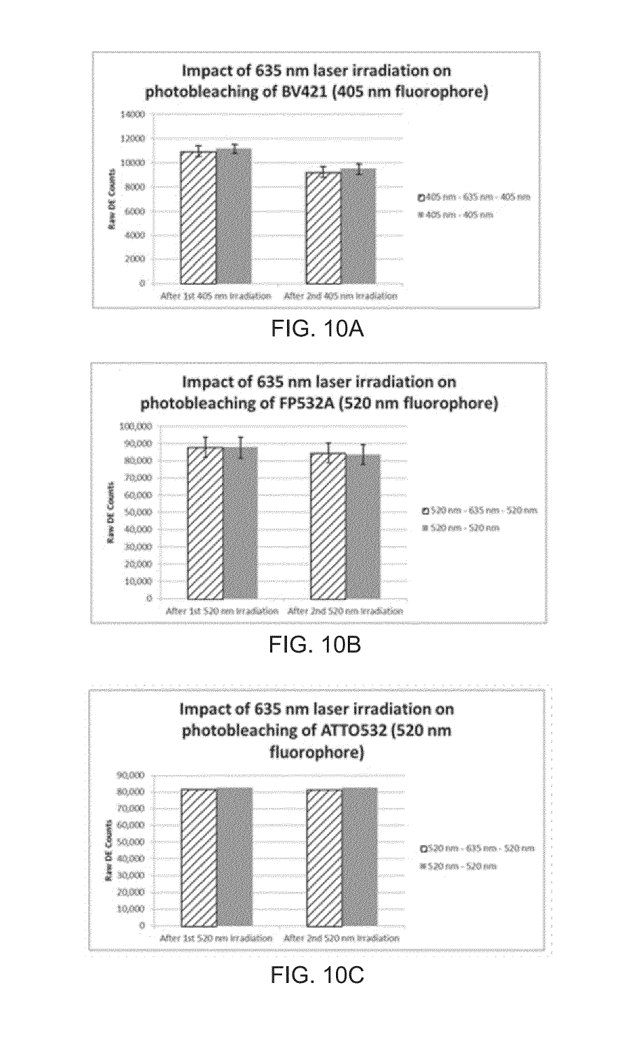

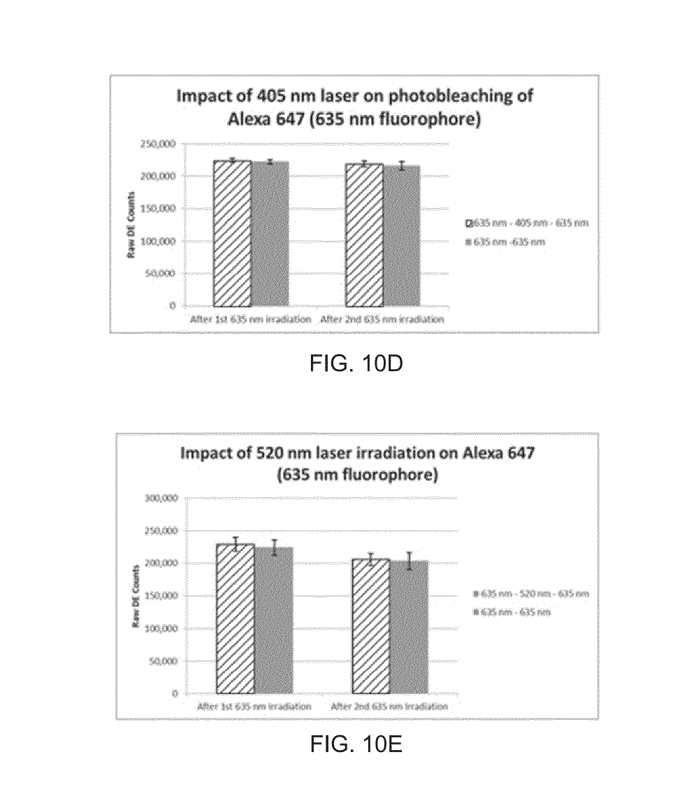

Many chemical fluorophores suffer from some degree of photobleaching, which may result in a loss of emission signal over time. In the multiplexed analyzer system of the disclosure, multiple wavelengths of energy at varying power levels may be directed at the labels in use. The selected fluorophores and electromagnetic radiation sources can be selected and optimized such that photobleaching over what occurs when a label is irradiated with its optimal (or approximately optimal) excitation wavelength is reduced or prevented. For example, where overlap between excitation and emission peaks of the labels is minimized, concurrent and sequential irradiation of tested labels may not cause photobleaching of the samples. FIGS. 10A-10E illustrate the results of experiments in which samples were irradiated with their primary laser, then either not irradiated or irradiated with a test laser. While some level of photobleaching may be expected due to sequential irradiation with the prime laser, the results shown in FIGS. 10A-10E do not exhibit a significant loss of signal when a fluorophore was irradiated by one of the lasers that was not optimal for that fluorophore. Additional photobleaching tests and examples are described below.

Example Multiplexed Single Molecule Analyzer Methods

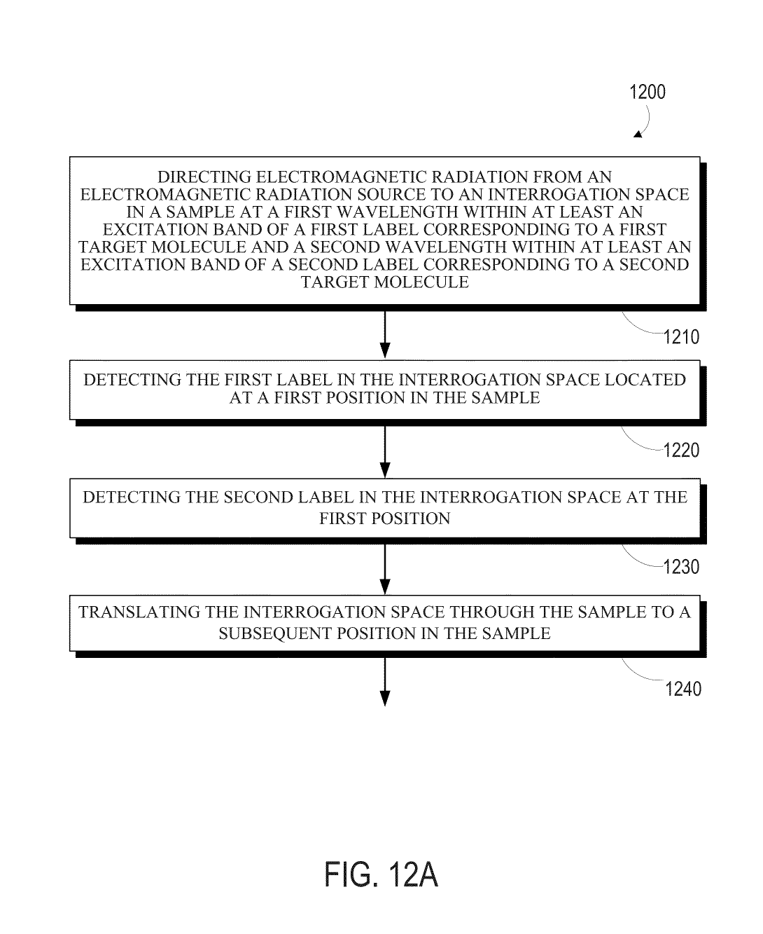

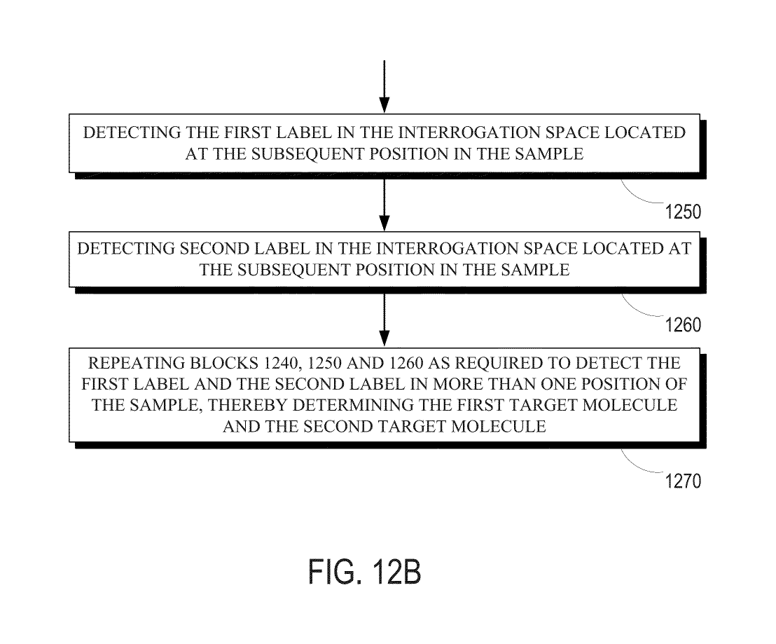

FIG. 12 is a flowchart of an example method 1200 for determining multiple target molecules. The method 1200 may, for example, be carried out with any analyzer capable of detecting multiple target molecule species, including any of the multiplexed analyzers described above. Electromagnetic radiation at a first wavelength and a second wavelength is directed from an electromagnetic radiation source to an interrogation space in a sample at block 1210. The first wavelength is within at least an excitation band of a first label corresponding to a first target molecule. The second wavelength is within at least an excitation band of a second label corresponding to a second target molecule. The first and the second labels are detected in the interrogation space at a first position in the sample at blocks 1220, 1230. The first and second labels may be detected simultaneously, for example, in an analyzer utilizing two detectors, or in series, for example, in an analyzer utilizing a single detector. The interrogation space is then translated through the sample to a subsequent position in the sample (at block 1240) and the first label and the second label are detected in the interrogation space at that subsequent position, either simultaneously or in series (at blocks 1250, 1260). Blocks 1240, 1250 and 1260 are repeated to detect the first label and the second label in more than one position of the sample, thereby determining the first target molecule and the second target molecule at block 1270. The interrogation space may, for example, be translated to multiple positions throughout the entire sample.

The first and second target molecules may be determined by, for example, a processor. The processor determines a first photon count signal from the first detector based, at least in part, on a photon count value for the first label detected in the interrogation space in each bin of a first plurality of bins. Each of the plurality of bins corresponds to the more than one position in the sample. The second photon count signal is determined from the second detector based, at least in part, on a photon count value for the second label detected in the interrogation space in each bin of a second plurality of bins. Each bin of the first plurality of bins that has a photon value for the first label greater than a first threshold value is used to identify instances of the first label in the interrogation space and thereby determine the first target molecule. The first threshold photon value for the first label corresponds to a background signal in the interrogation space at an emission wavelength of the first label in at least one position in the sample. The second target molecule is determined by counting instances of the second label in the interrogation space by identifying each bin of the second plurality of bins having a photon value for the second label greater than a second threshold value. The second threshold photon value for the second label corresponds to a background signal in the interrogation space at an emission wavelength of the second label in at least one position in the sample.

In some embodiments, electromagnetic radiation at the first wavelength and electromagnetic radiation at the second wavelength are directed to the interrogation space in series. The first label is detected when electromagnetic radiation at the first wavelength is directed to the interrogation space and the second label is detected when electromagnetic radiation at the second wavelength is directed to the interrogation space. An analyzer having a single source of broadband electromagnetic radiation or multiple sources, such as a first source that provides electromagnetic radiation at the first wavelength and second source that provides electromagnetic radiation at the second wavelength, may be used. In some cases, a single detector may be used. The first plurality of bins may be different than the second plurality of bins. Additionally, the method may be carried out with an analyzer that includes a first detector for detecting the first label and a second detector for detecting the second label, wherein the first detector does not detect an emission wavelength of the second label and the second detector does not detect an emission wavelength of the first label.

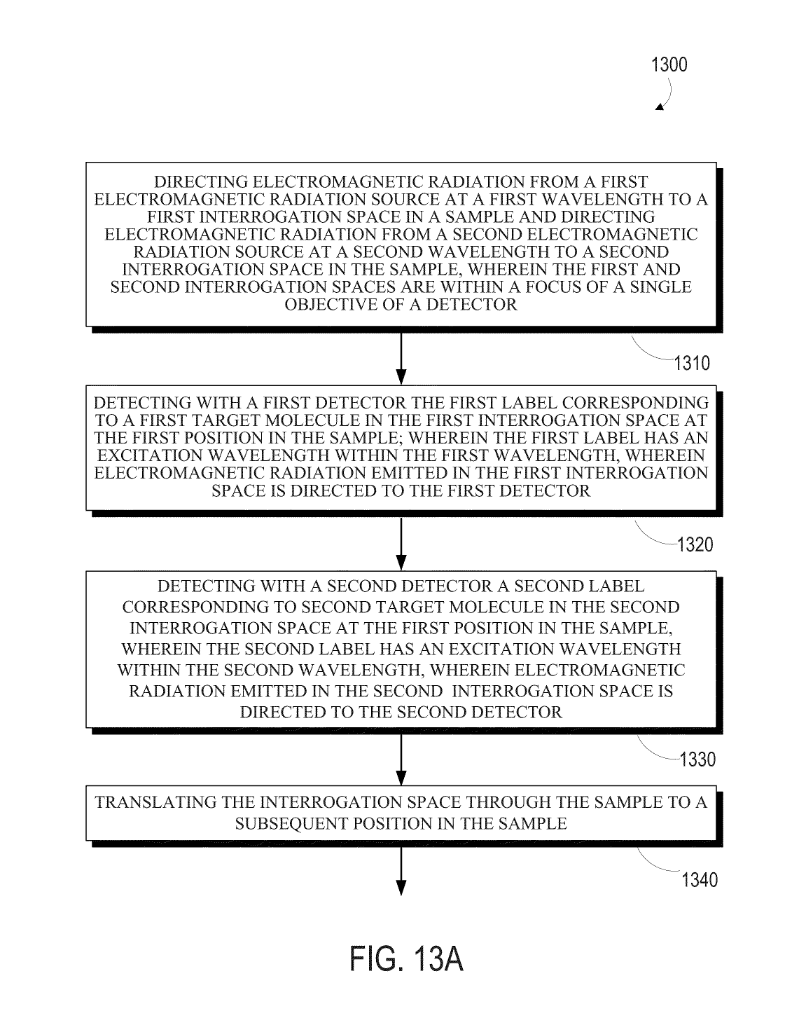

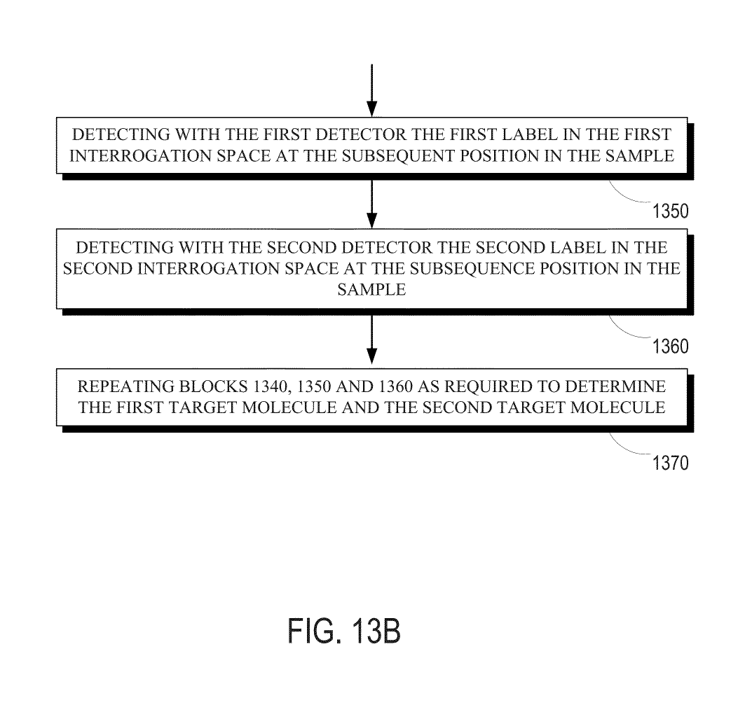

FIG. 13 is a flowchart of an example method 1300 for determining multiple target molecules. The method 1300 may, for example, be carried out with any analyzer capable of detecting multiple target molecule species, including any of the multiplexed analyzers described above. Electromagnetic radiation at a first wavelength emitted from a first electromagnetic radiation source and electromagnetic radiation at a second wavelength emitted from a second electromagnetic radiation source are directed to a first interrogation space and a second interrogation space in the sample, respectively at block 1310. The first and second interrogation spaces are within a focus of a single objective of a detector. A first detector detects the first label having an excitation wavelength within the first wavelength and corresponding to a first target molecule in the first interrogation space at the first position in the sample at block 1320. Electromagnetic radiation emitted in the first interrogation space is directed to the first detector. A second detector detects a second label having an excitation wavelength within the second wavelength and corresponding to second target molecule in the second interrogation space at the first position in the sample at block 1330. Electromagnetic radiation emitted in the second interrogation space is directed to the second detector.

The interrogation space is then translated through the sample to a subsequent position in the sample (at block 1340) where the first label in the first interrogation space and the second label in the second interrogation space are detected with the first and the second detectors, respectively at blocks 1350, 1360. Blocks 1340, 1350 and 1360 are repeated as required to determine the first target molecule and the second target molecule.

A first photon count signal output from the detector comprising a photon count value for the first label detected in the interrogation space is determined, for example, by a processor in each bin of a first plurality of bins. A second photon count signal output from the second detector comprising a photon count value for the second label detected in the interrogation space is also determined in each bin of a second plurality of bins. Each bin of the first and the second plurality of bins correspond to the more than one position in the sample. The first target molecule is determined by counting instances of the first label in the interrogation space by identifying each bin of the first plurality of bins having a photon value for the first label greater than a first threshold value. The first threshold photon value for the first label corresponds to a background signal in the interrogation space at an emission wavelength of the first label in at least one position in the sample. The second target molecule is determined by counting instances of the second label in the interrogation space by identifying each bin of the second plurality of bins having a photon value for the second label greater than the second threshold value. The second threshold photon value for the second label corresponds to a background signal in the interrogation space at an emission wavelength of the second label in at least one position in the sample.

### EXAMPLES

### Example 1

### Molecule Detection and Standard Curve Generation

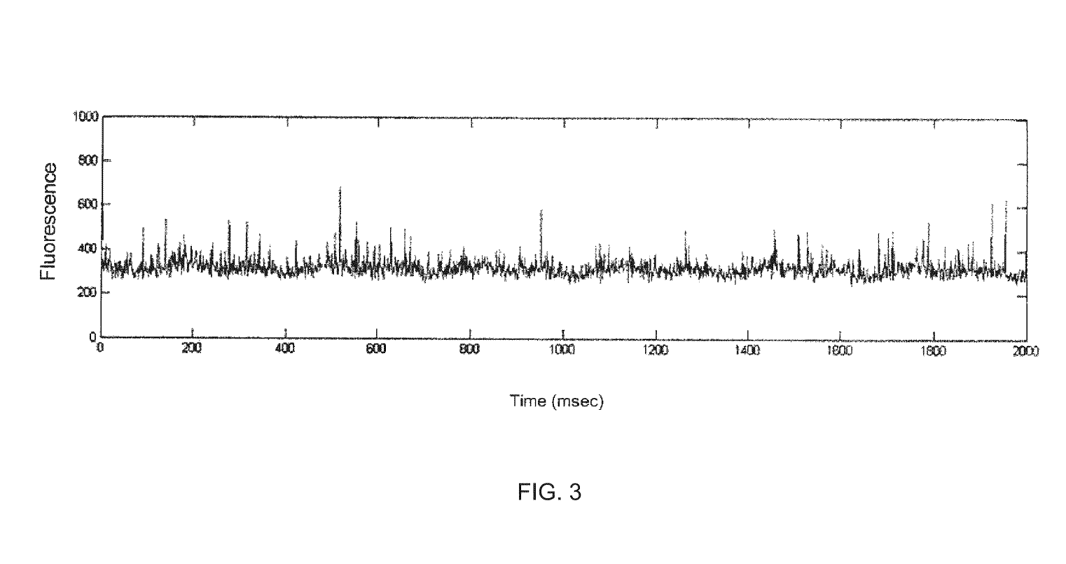

FIG. 3 illustrates the detection of single molecules using a device of the disclosure. The plot shows representative data for fluorescence detected on the vertical axis versus time (msec) on the horizontal axis. The spikes shown in the graph were generated when the scanning single molecule analyzer encountered one or more labeled molecules within the interrogation space. The total fluorescent signal comprises the sum of individual detection events (DE). The count of all the detection events during the recording can be referred to as the “DE value.” As described above, at low concentrations, the DE value corresponds to the number of detected molecules. At higher concentrations wherein two or more molecules can pass through the detection spot at once, the number of molecules detected can be higher than the DE count.

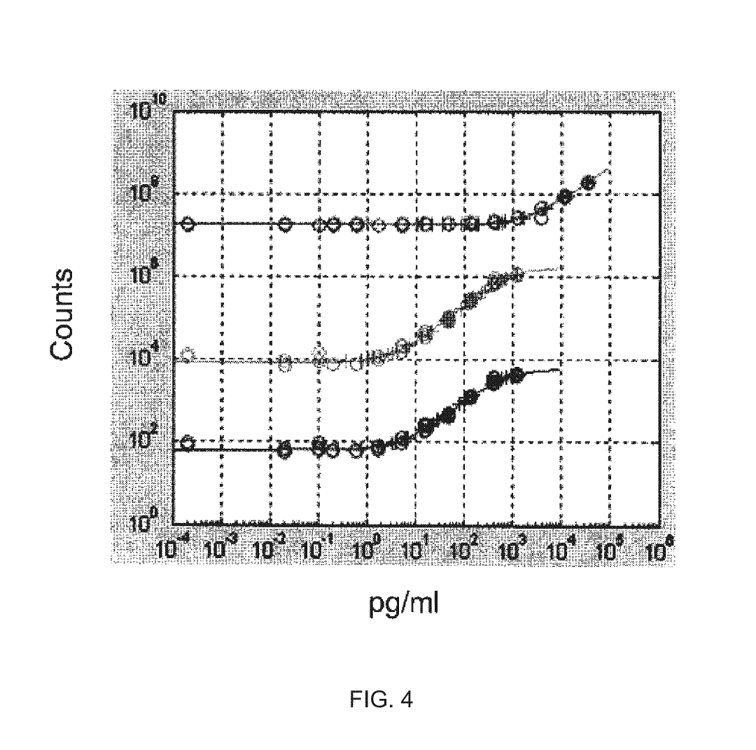

FIG. 4 illustrates a standard curve generated with a scanning single molecule analyzer. To generate the curve, samples were prepared with known concentrations and measured using a device of the disclosure. Three curves are shown in the plot. The upper curve corresponds to the total photons (TP) detected. The middle curve corresponds to the event photons (EP) detected. The lower curve corresponds to detected events (DE). The plot shows the values for each of these measures (“Counts”) on the vertical axis versus the known sample concentration (pg/ml) on the horizontal axis. The plotted circles are the counts plotted at their known concentrations. The solid curve is a least squares fit of the data to a four parameter logistics curve. The “+” symbols are the counts plotted at their interpolated concentrations instead of their known concentrations. The “+” symbols indicate how well the fitted curve passes through the actual data. This data demonstrates that as the concentration of the sample is varied, there is a clear change in the number of molecules detected.

### Example 2

### Sandwich Assays for Biomarkers: Cardiac Troponin I (cTnI)

The Assay: The purpose of this assay is to detect the presence of cardiac Troponin I (cTNI) in human serum. The assay format comprises a two-step sandwich immunoassay using a mouse monoclonal capture antibody and a goat polyclonal detection antibody. Ten microliters of sample are required. The working range of the assay is 0-900 pg/ml with a typical analytical limit of detection of 1 to 3 pg/ml. The assay requires about 4 h of bench time to complete.

Materials: The following materials are used in the procedure described below. The assay plate comprises a clear 384 well NUNC™ Maxisorp, product 464718. The plate is passively coated overnight at room temperature with a monoclonal antibody comprising BiosPacific A34440228P Lot # A0316 (5 μg/ml in 0.05 M sodium carbonate pH 9.6) and blocked with 5% sucrose, 1% BSA in phosphate buffered saline (PBS), and stored at 4° C. For the standard curve, Human cardiac Troponin I (BiosPacific Cat #J34000352) is used. The diluent for the standard concentrations is human serum immuno-depleted of endogenous cTNI, aliquoted and stored at −20° C. Standards are diluted in a 96 well, conical, polypropylene plate (NUNC™ product #249944). The following buffers and solutions are used: (a) assay buffer (borate buffer saline (BBS) with 1% BSA and 0.1% Triton X-100); (b) passive blocking solution (assay buffer containing 2 mg/ml mouse IgG (Equitech Bio), 2 mg/ml goat IgG (Equitech Bio), and 2 mg/ml MAK33 IgG1 Poly (Roche #11 939 661)); (c) detection antibody (goat polyclonal antibody affinity purified to Peptide 3 (BiosPacific G-129-C), labeled with fluorescent dye ALEXA FLUOR® 647, and stored at 4° C.); (d) detection antibody diluent (50% assay buffer, 50% passive blocking solution); (e) wash buffer (borate buffer saline Triton buffer (BBST) (1.0 M borate, 15.0 M sodium chloride, 10% Triton X-100, pH 8.3)); (f) elution buffer (BBS with 4M urea, 0.02% Triton X-100 and 0.001% BSA); and (g) coupling buffer (0.1 M NaHCO₃).

Preparation of ALEXA FLUOR® 647 Labeled Antibodies: The detection antibody G-129-C is prepared by conjugation to ALEXA FLUOR® 647. 100 μg of G-129-C is dissolved in 400 μl of the coupling buffer. The antibody solution is concentrated to 50 μl by transferring the solution into YM-30 filter and subjecting the solution and filter to centrifugation. The YM-30 filter and antibody are washed three times by adding 400 μl of the coupling buffer. The antibody is recovered by adding 50 μl of coupling buffer to the filter, inverting the filter, and centrifuging for 1 min at 5,000×g. The resulting antibody solution has a concentration of about 1-2 μg/μl. ALEXA FLUOR® 647 NHS ester stock solution is made by reconstituted one vial of ALEXA FLUOR® 647 in 20 μl DMSO. This solution can be stored at −20° C. for up to 1 month. 3 μl of ALEXA FLUOR® 647 stock solution is mixed with the antibody solution in the dark for 1 h. Thereafter, 7.5 μl 1 M tris is added to the antibody ALEXA FLUOR® 647 solution and mixed. The solution is ultrafiltered with YM-30 to remove low molecular weight components. The volume of the retentate, which contains the antibody conjugated to ALEXA FLUOR® 647, is adjusted to 200-400 μl by adding PBS. 3 μl 10% NaN₃ is added to the solution. The resulting solution is transferred to an Ultrafree 0.22 centrifugal unit and centrifuged for 2 min at 12,000×g. The filtrate containing the conjugated antibody is collected and used in the assays.

Procedure: Standards are prepared (0-900 pg/ml) by serial dilutions of the stock of cTnI standard into standard diluent to achieve a range of cTnI concentrations of between 1.2 pg/ml-4.3 μg/ml. 10 μl passive blocking solution and 10 μl of either the standard or a sample are added to each well of the appropriate plate. Standards are run in quadruplicate. The plate is sealed, preferably with a low-fluorescence sealing film, centrifuged for 1 min at 3000 RPM, and incubated for 2 h at 25° C. with shaking. The plate is washed five times, and centrifuged until the rotor reaches 3000 RPM in an inverted position over a paper towel. A 1 nM working dilution of detection antibody is prepared, and 20 μl detection antibody are added to each well. The plate is sealed and centrifuged, and the assay is incubated for 1 h at 25° C. with shaking. 30 μl elution buffer are added per well, the plate is sealed and the assay is incubated for ½ h at 25° C. The plate can be analyzed immediately or can be stored for up to 48 h at 4° C. prior to analysis.

For analysis, 20 μl per well are acquired at 40 μl/minute, and 5 μl are analyzed at a 16.7 mm/sec scan rate. The data is analyzed based on a threshold of 4 standard deviations (σ). The raw signal is plotted versus concentration of the standards. A linear fit is performed for the low concentration range, and a non-linear fit is performed for the full standard curve. The limit of detection (LOD) is calculated as LOD=(3×σ of zero samples)/slope of linear fit. The concentrations of the samples are determined from the linear or non-linear equation appropriate for the sample signal.

The sample plate is then loaded into the scanning single molecule analyzer. Individually-labeled antibodies are measured by translating the interrogation space through the sample at a speed such that the emission from only one fluorescent label is detected in a defined space following laser excitation. The total fluorescent signal is a sum of the individual detection events as described above.

### Example 3

### Sandwich Bead-Based Assays for TnI

The assays described above uses a microtiter plate format where the plastic surface is used to immobilize target molecules. The single particle analyzer system is also compatible with assays performed in solution using microparticles or beads to separate bound and unbound entities.

Materials: MyOne Streptavidin C1 microparticles (MPs) are obtained from Dynal (650.01-03, 10 mg/ml stock). Buffers used include: (a) 10× borate buffer saline Triton Buffer (BBST) (1.0 M borate, 15.0 M sodium chloride, 10% Triton X-100, pH 8.3); (b) assay buffer (2 mg/ml normal goat IgG, 2 mg/ml normal mouse IgG, and 0.2 mg/ml MAB-33-IgG-Polymer in 0.1 M Tris (pH 8.1), 0.025 M EDTA, 0.15 M NaCl, 0.1% BSA, 0.1% Triton X-100, and 0.1% NaN₃, stored at 4° C.); and (c) elution buffer (BBS with 4 M urea, 0.02% Triton X-100, and 0.001% BSA, stored at 2-8° C.). Antibodies used in the sandwich bead-based assay include: (a) Bio-Ab (A34650228P (BiosPacific) with 1-2 biotins per IgG); and (b) Det-Ab (G-129-C (BiosPacific) conjugated to ALEXA FLUOR® 647, 2-4 fluors per IgG). The standard is recombinant human cardiac troponin I (BiosPacific, cat 034120352). The calibrator diluent is 30 mg/ml BSA in tris buffered saline (TBS) with EDTA.

Microparticles Coating: 100 μl of the MPs stock solution is placed in an Eppendorf tube. The MPs are washed three times with 100 μl BBST wash buffer by applying a magnet, removing the supernatant, removing the magnet, and resuspending in wash buffer. After washing, the MPs are resuspended in 100 μl of assay buffer and 15 μg of Bio-Ab are added. The mixture is incubated for 1 h at room temperature with constant mixing. The MPs are washed five times with 1 ml wash buffer as described above. After the washes the MPs are resuspended in 15 ml of assay buffer (or 100 μl to store at 4° C.).

Preparation of Standard and Samples: The standard is diluted with calibrator diluent to prepare a proper standard curve, typically ranging from 200 pg/ml to 0.1 pg/ml. Frozen serum and plasma samples are centrifuged 10 min at room temperature at 13,000 rpm. Clarified serum or plasma is removed carefully to avoid pellets or floaters and transferred to fresh tubes. 50 μl of each standard or sample is pipetted into appropriate wells.

Capture Target: After resuspension to 15 ml in assay buffer comprising 400 mM NaCl, 150 μl of the MPs are added to each well. The mixture is incubated on a Boekel Jitterbug Microplate Incubator Shaker at room temperature for 1 h.

Washes and Detection: The plate is placed on a magnet and the supernatant is removed after allowing the magnets to capture the MPs. After removing the plate from the magnet, 250 μl of wash buffer are added. Again, the plate is placed on a magnet and the supernatant is removed after allowing the magnets to capture the MPs. 20 μl Det-Ab are added per well. If necessary, Det-Ab to 500 ng/ml is first diluted in assay buffer comprising 400 mM NaCl. The mixture is incubated on a Boekel Jitterbug Microplate Incubator Shaker at room temperature for 30 min. The plate is washed as described three times with wash buffer. After washing, 250 μl of wash buffer are added and the samples are transferred into a new 96-well plate. The wash step is repeated twice. 20 μl of elution buffer are then added and the mixture is incubated on Boekel Jitterbug Microplate Incubator Shaker at room temperature for 30 min.

Filter MPs and Transfer to 384-well Plate: The standard and samples are transferred into a 384-well filter plate placed on top of a 384-well assay plate. The plate is centrifuged at room temperature at 3000 rpm. The filter plate is removed and the appropriate calibrators are added. The plate is covered and is ready for scanning single molecule detector.

Scanning Single Molecule Detector: A sample in a sample well is scanned using an electromagnetic radiation source. The interrogation space is translated through the sample. The sample is scanned at a speed that is sufficiently slow so that individually-labeled antibodies are measured during the sample scan. This is achieved by setting the interrogation space such that the emission of only one fluorescent molecule, if present, is detected in a defined space following laser excitation. With each signal representing a digital event, this configuration enables extremely high analytical sensitivities. Total fluorescent signal is determined as a sum of the individual digital events. Each molecule counted is a positive data point with hundreds to thousands of detected events/sample. The limit of detection the cTnI assay of the disclosure is determined by the mean plus 3 σ method (see above).

### Example 4

### Electromagnetic Radiation Source, Filter and Fluorophore Combinations

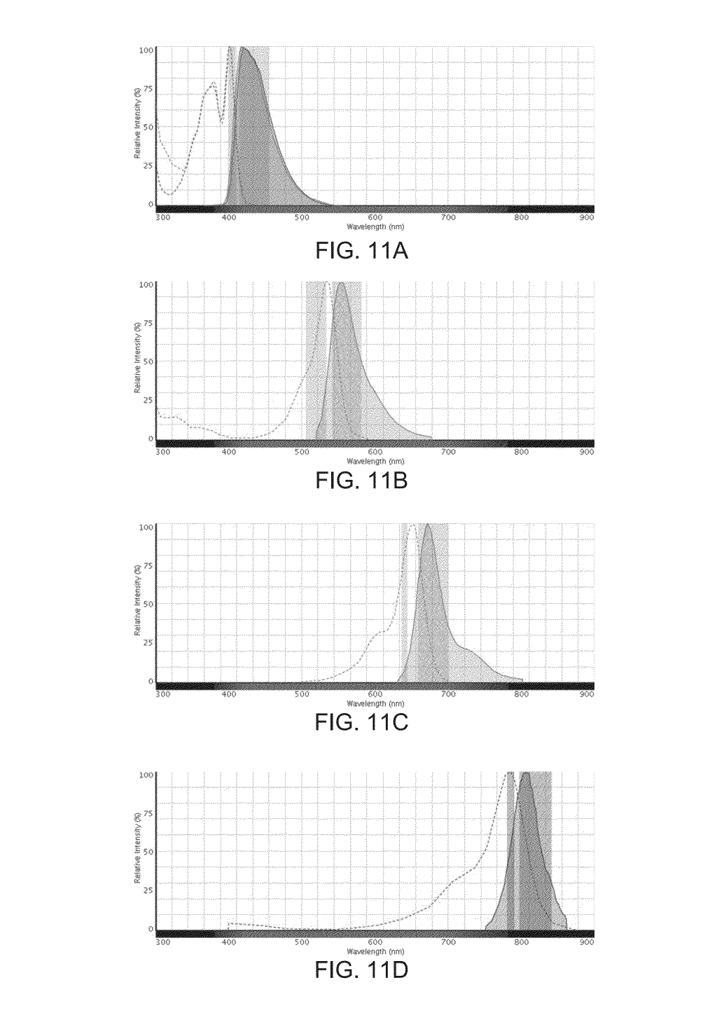

Exemplary excitation and emission filters that can be used with excitation lasers of 405 nm, 520 nm, 635 nm and 785 nm are charted in Table 2. FIGS. 11A-11D illustrate the excitation and emission spectra for five different fluorophores—ALEXA FLUOR® 405 and Cascade Blue (11A), ALEXA FLUOR® 532 (11B), ALEXA FLUOR® 647 (11C) and ALEXA FLUOR® 790 (11D)—when the electromagnetic radiation sources, excitation filters and emission filters of Table 2A are used. The dotted lines indicate the excitation spectra and the solid lines indicate the emission spectra. The respective filter wavelengths and bandwidths are also shown.

TABLE 2
Excitation Lasers, Excitation Filters and Emission Filters

| | Excitation filter | Emission filter |
|---|---|---|
| Excitation Laser | (wavelength, bandwidth) | (wavelength, bandwidth) |
| 405 nm | 405 nm, 10 nm | 435 nm, 40 nm |
| 520 nm | 520 nm, 28 nm | 562 nm, 40 nm |
| 635 nm | 640 nm, 8 nm | 679 nm, 41 nm |
| 785 nm | 785 nm, 10 nm | 819 nm, 44 nm |

The percent of signal bleedthrough of each fluorophore excited by different lasers with the filters listed in Table 1 in place is shown in Table 2. No more than 1% bleedthrough of any signal is predicted under these conditions.

TABLE 2B
Signal Bleedthrough

| Fluorophore/Filter | 435/40 | 562/40 | 679/41 | 819/44 |
|---|---|---|---|---|
| ALEXA FLUOR ® 405 | 62.8% | −0.4% | 0.0% | 0.0% |
| Cascade Blue | 60.7% | 0.2% | 0.0% | 0.0% |
| ALEXA FLUOR ® 532 | 0.0% | 59.4% | 1.0% | 0.0% |
| Alexa Fuor 647 | 0.0% | 0.0% | 60.1% | 0.2% |
| ALEXA FLUOR ® 790 | 0.0% | 0.0% | 0.0% | 61.0% |

The following combinations of fluorophores exhibit minimal excitation overlap: Excitation at 405 nm: Brilliant Violet 421, Alexa 405, Cascade Blue, DyLight 405; Excitation at 520 nm: ATTO532, FluoProbe 532A, DY 530, CF532; Excitation at 635 nm: ALEXA FLUOR® 647, DY647, DY648, ATTO 647; Excitation at 785 nm: ALEXA FLUOR® 790, CF790, DY800, FluoProbe 582. In one embodiment, the combination of Brilliant Violet 421, ATTO532, and ALEXA FLUOR® 647 is used.

### Example 5

### Evaluation of Example Multiplexed Analyzer System

To test the functionality of the multiplexed analyzer system, a model assay system containing IL-4, IL-6 and IL-10 was developed. These three analytes were chosen to test the multiplexed analyzer system's capability to accurately and reproducibly measure concentrations over four orders of magnitude without dilution of complex plasma samples.

The model system included multiple electromagnetic radiation sources and multiple detectors in an arrangement similar to that shown in FIG. 7B. In particular, the electromagnetic radiation sources included three fiber-optically coupled lasers at 405 nm, 520 nm, and 635 nm. The excitation ranges of these three lasers were far enough apart that traditional fluorophores excited by the lasers had minimal emission overlap. By employing separation of emission wavelengths, separate detectors can be used to monitor the same spot, with filters in the optical path to ensure that each detector only receives photons from the corresponding label type (see FIG. 7B). Three lasers were used in this example so that the power levels could be tuned independently for each laser source to optimize fluorescence signal and minimize photobleaching for each fluorophore used. The three lasers could also be uniquely collimated and focused using a confocal objective lens either onto the same spot or onto unique spots within the interrogation space of the sample.

Responsive to the lasers providing electromagnetic radiation to the sample, fluorescence from samples was emitted and emission filters and dichroic mirrors reflected and filtered out any stray excitation light before the signals were transmitted via fiber optic cables to the detectors. Two types of detectors were employed within the example system. For maximum efficiency for the detection of fluorophores excited with either the 520 nm or 635 nm lasers, a single photon counting module from Excelitas™ was employed with between 60-70% photon detection efficiency for the emissions of these fluorophores. For maximum photon detection efficiency for the fluorophores used with the 405 nm laser, a Hamamatsu™ detector was employed with approximately 30-40% photon detection efficiency.

To improve electromagnetic excitation and signal detection, the objective lens included an optical coating that allowed efficient transmission of photons from lasers in the range of 405 nm-635 nm as well as photons emitted from fluorescent samples in the range of 421 nm-647 nm. The collimators used after the lasers and before the detectors were also specific for the wavelengths of the laser to minimize signal loss. Fiber optic connections were used between all laser outputs and the collimators and collimators and detectors.

All lasers, detectors were individually focused and aligned to a single spot for each laser/detector combination within the sample volume (32 μl total within a 384 well plate well) starting with the 405 nm laser, then the 520 nm laser and finally the 635 nm laser. The power level for the 635 nm laser was set to 5 mW. The 520 nm laser was also set at 5 mW which gave the best signal to noise ratio. The 405 nm laser was set at 1.2 mW to balance signal intensity with photobleaching of the 405 nm absorbing fluorophore BV421.

Evaluation of Excitation Cross-Talk and Photobleaching

In one aspect, the impact of each laser on the photostability and the emission signal of each of the fluorophores were evaluated. For purposes of this test, fluorophores of BV421, ATTO532, FP532A, and Alexa 647 were used. These fluorophores were expected to not be significantly excited, and hence have minimal detectable emission, if a laser far from the excitation maximum of the fluorophore was used. A corresponding laser was selected for each fluorophore such that the laser's wavelength was approximately at or near the excitation maximum of the corresponding fluorophore. The corresponding laser used for BV421 excitation was the 405 nm laser, the corresponding laser used for ATTO532 and FP532A was the 520 nm laser, and the corresponding laser used for Alexa 647 was the 635 nm laser.

Assuming that no excitation would occur due to non-corresponding lasers, then no photobleaching was expected to occur either. These assumptions were tested experimentally by comparing the emission of solutions of fluorophores conjugated to proteins at concentrations where single molecules can be counted after irradiation by corresponding and non-corresponding lasers. Tables 3A-3B below show the impact of excitation by each laser on each fluorophore. As shown in Tables 3A-3B, it was found that there was no significant difference in the signal between a buffer blank and a fluorophore covalently linked to a protein when the fluorophores were irradiated by a laser that was far away from its absorption maxima.

It was also desired that irradiation with a laser at the non-excitation peak did not cause another form of energy absorption that could lead to molecular emission loss, photobleaching, or some other form of signal decay. The loss of signal was measured by irradiating fluorophore-labeled proteins first with their corresponding excitation laser, then a subset of replicates were irradiated with a non-peak absorption laser, while another subset of samples were not irradiated and finally all the samples were irradiated again with the corresponding excitation laser. Some photobleaching/signal loss was expected from two irradiation steps with the corresponding excitation laser since none of the fluorophores are completely photostable and a high level of laser energy was being directed at a very small volume of sample. The amount of signal loss due to each laser irradiation step is shown in Tables 3A-3B.

In particular, to conduct the test, 24 μl of 1000 pg/ml IL-4 ATTO532, IL-6 Alexa 647 samples or streptavidin BV421 in a 0.25 M Tris, 0.05M glycine, 0.01% Triton-X-100 solution, pH 8 were aliquoted into the wells of a 384-well Greiner plate. Ten replicates were irradiated by the corresponding laser, followed by a non-corresponding laser, and then by a corresponding laser. Ten replicates were irradiated by only the corresponding laser followed by a second irradiation by the corresponding laser. The average single molecule counts after the first irradiation step by the corresponding laser and the second irradiation step by the corresponding laser were calculated for both sets of samples. Tukey-Kramer group comparisons were used to determine whether the null hypothesis at the 5% significance level could be rejected for all pairs. This analysis determines that the samples all give consistent signals after one irradiation using the corresponding laser, if a significant loss of signal occurs due to irradiation with the corresponding laser twice (standard photobleaching), and if more signal loss occurs if samples are irradiated with a non-corresponding laser along with two irradiation steps by the corresponding laser (other forms of fluorescence loss besides photobleaching). The 405 nm laser was run at 1.2 mW power and the 520 nm and 635 nm lasers were run at 5 mW power for these assays

Table 3A below shows a comparison of fluorophore signal when irradiated with a non-excitation optimal laser versus a buffer blank irradiated with the same laser by Tukey-Kramer analysis at a 95% confidence level resulted in no significant differences between signals at p<0.0001. Table 3B below shows the impact on single molecule counts and photobleaching of fluorophores. As noted above, samples were irradiated multiple times either in a sequence of corresponding laser/non-corresponding laser/corresponding laser or just irradiated twice by the corresponding laser. Differences were detected by Tukey-Kramer group analysis at a 95% confidence level.

TABLE 3A

| | Mean Raw | Mean Raw | Mean Raw |
|---|---|---|---|
| | DE for 1,000 | DE for 1,000 | DE for 1,000 |
| Fluorophore | pg/ml using | pg/ml using | pg/ml using |
| labeled protein | 405 nm laser | 520 nm laser | 635 nm laser |
| IL-4 ATTO532 | 111^{a} | 179,939 | 110^{a} |
| IL-6 Alexa 647 | 111^{a} | 378^{a} | 250,535 |
| Streptavidin | 103,838 | 457^{a} | 132^{a} |
| BV421 | | | |
| Buffer Blank | 115^{a} | 239^{a} | 126^{a} |

^{a}Do not reject the null hypothesis that these samples have the same means at a 5% significance level for samples irradiated using the same laser

TABLE 3B

| | | Average raw DE | Average raw DE | % Loss of |
|---|---|---|---|---|
| | | counts after first | counts after second | raw DE |
| Fluorophore | Laser irradiation cycle | irradiation | irradiation | signal |
| Alexa 647 | 635 nm laser - 635 nm | 240,676 ± 3,220 | 227,780 ± 4,241^{a,b} | 5.4% |
| | laser | | | |
| Alexa 647 | 635 nm laser - 405 nm | 241,735 ± 3,643 | 227,842 ± 3,793^{a,b} | 5.7% |
| | laser - 635 nm laser | | | |
| Alexa 647 | 635 nm laser - 520 nm | 242,018 ± 3,816 | 225,124 ± 4,156^{a,b} | 7.0% |
| | laser - 635 nm laser | | | |
| ATTO532 | 520 nm laser - 520 nm | 173,485 ± 4,361 | 174,273 ± 6,530^{b} | −1.2% |
| | laser | | | |
| ATTO532 | 520 nm laser - 635 nm | 170,462 ± 2,517 | 173,564 ± 2,837^{b} | −0.5% |
| | laser - 520 nm laser | | | |
| ATTO532 | 520 nm laser - 405 nm | 171,152 ± 1,739 | 173,326 ± 1,964^{b} | −0.5% |
| | laser - 520 nm laser | | | |
| BV421 | 405 nm laser - 405 nm | 115,570 ± 2,652 | 100,982 ± 4,112^{a,b} | 12.6% |
| | laser | | | |
| BV421 | 405 nm laser - 520 nm | 115,839 ± 2,589 | 100,677 ± 4,969^{a,b} | 13.1% |
| | laser - 405 nm laser | | | |
| BV421 | 405 nm laser - 635 nm | 115,224 ± 2,688 | 102,921 ± 4,873^{a,b} | 10.7% |
| | laser - 405 nm laser | | | |

^{a}p < 0.001 for first irradiation versus second irradiation
^{b}Do not reject the null hypothesis at the 5% significance level for optimal-optimal samples after second irradiation being different than optimal-non-optimal-optimal samples after second irradiation

TABLE 3C

| | Fluor MW | Protein MW | Total mass for DoL = 3 |
|---|---|---|---|
| IL-4 ATTO532 | 1,250 | 150,000 | 153,750 |
| IL-6 Alexa 647 | 1,000 | 150,000 | 153,000 |
| Streptavidin BV421 | 70,000 | 54,000 | 264,000 |

For the photobleaching studies, it can be seen that multiple irradiations of Alexa 647 with the 635 nm laser caused photobleaching of the fluorophore between with a loss of 5-7% of the single molecule counts. An additional irradiation step with either the 405 nm laser or 532 nm laser did not significantly impact the level of signal loss for Alexa 647 versus multiple irradiations with just the 635 nm laser. The ATTO532 fluorophore was the most stable and showed no significant loss in signal with either multiple irradiation or multiple irradiation plus an irradiation with either the 405 nm or 635 nm lasers. The BV421 fluorophore also showed signal loss of 12.6% with multiple irradiations by the 405 nm laser but this signal loss was not increased by irradiation by either the 520 nm or 635 nm lasers. Therefore, it was concluded that while the fluorophores might experience some degree of photoinstability/signal loss if irradiated multiple times with their corresponding lasers, irradiation with a non-corresponding laser does not significantly impact signal intensity. Typical experiments do not involve multiple irradiation steps with the corresponding laser.

Evaluation of Assay Reagent Cross-Reactivity

For multiplexing to give valid results, it may be beneficial to reduce, limit, or eliminate cross-reactivity between the different antigens and antibodies for non-specific partners. The cross reactivity of the IL-4, IL-6 and IL-10 reagents were tested by comparing the signals for 3-PLEXes (i.e., multiplex assays) run using all three capture antibody, one antigen and all three detection antibodies and excitation at 405 nm, 520 nm, and 635 nm. These results were compared to the values from running the assays as 1-PLEXes (i.e., singleplex assays) with one capture antibody, antigen and detection antibody. The results of the cross-reactivity assays are summarized in Table 4.

TABLE 4
Cross-reactivity of IL-4, IL-6 and IL-10 assays

| | 1-PLEX or 3- | | | Blank Raw DE |
|---|---|---|---|---|
| Fluorophore | PLEX | Laser | Slope | Counts |
| IL-4 Alexa 647 | 3-PLEX | 405 nm | −6 | 522 |
| IL-4 Alexa 647 | 3-PLEX | 520 nm | −87 | 3,523 |
| IL-4 Alexa 647 | 3-PLEX | 635 nm | 7,588 | 627 |
| IL-4 Alexa 647 | 1-PLEX | 405 nm | 2 | 79 |
| IL-4 Alexa 647 | 1-PLEX | 520 nm | −3 | 496 |
| IL-4 Alexa 647 | 1-PLEX | 635 nm | 11,659 | 466 |
| IL-6 ATTO532 | 3-PLEX | 405 nm | 3 | 516 |
| IL-6 ATTO532 | 3-PLEX | 520 nm | 2193 | 3,430 |
| IL-6 ATTO532 | 3-PLEX | 635 nm | 0 | 575 |
| IL-6 ATTO532 | 1-PLEX | 405 nm | 0 | 59 |
| IL-6 ATTO532 | 1-PLEX | 520 nm | 2,755 | 527 |
| IL-6 ATTO532 | 1-PLEX | 635 nm | −1 | 283 |
| IL-10 BV421 | 3-PLEX | 405 nm | 1,750 | 415 |
| IL-10 BV421 | 3-PLEX | 520 nm | 1 | 3,644 |
| IL-10 BV421 | 3-PLEX | 635 nm | 0 | 560 |
| IL-10 BV421 | 1-PLEX | 405 nm | 2,111 | 803 |
| IL-10 BV421 | 1-PLEX | 520 nm | −2 | 679 |
| IL-10 BV421 | 1-PLEX | 635 nm | −1 | 277 |

In Table 4, the slope values show the antigen concentration dependence of the assay. It was expected to be close to zero as long as there is no cross-reactivity between the capture and detection antibodies added that are not specific for the particular antigen added. The blank raw DE counts are a combination of signal due to instrumental and electrical noise as well as non-specific binding of detection antibodies (not dose dependent) to wells, antigens and other antibodies. The lower the value for the blank raw DE counts, the better.

For the emission signals where the fluorophores were excited by the corresponding laser for the assay being tested (405 nm laser for BV421, 520 nm laser for ATTO532 and 635 nm laser for Alexa 647), for both 1-PLEX and 3-PLEX assays there were large slopes indicating antigen dose dependence and specific signal. The signals for the fluorophore labeled detection antibodies not specific for the one antigen added for the 3-PLEX cross-reactivity assays had slopes close to zero and hence did not show any dose dependence in signal so there was no cross-reactivity occurring between detection antibodies and antigens of the IL-4, IL-6 and IL-10 assays. It should be noted that the background signals due to the IL-6 ATTO532 irradiated with the 520 nm laser in the IL-4 and IL-10 assays as well as the specific IL-6 assay did give large baseline values. This is indicative of a larger degree of non-specific binding of the IL-6 detection antibody within the 3-PLEX since this large background is not seen in the 1-PLEX IL-6 ATTO532 assay. This large background can potentially be decreased with more optimization of detection antibody concentrations, levels of surfactant in assay buffers, or number of wash cycles.

Comparison of Multiplexed Assays and Singleplexed Assays

As noted above running multiplexes can save time and save sample volumes; however, it may desirable that such benefits do not come at the expense of reproducibility and sensitivity. It also may be desirable to have flexibility as to which fluorophore is used for detection of which analyte. Each fluorophore has advantages and challenges. For example, BV421 has the highest extinction coefficient but can suffer from steric issues being almost 70× larger than other fluorophores and also is less photostable. Additionally, for example, Alexa 647 has a high extinction coefficient and is generally quite stable but has a lower quantum yield. Further, for example, Green fluorophores such as ATTO532 have the lowest extinction coefficients of the fluorophores tested, but also are extremely stable and have very high quantum efficiencies.

The multiplex analyzer system was tested using three different labeling schemes for the detection antibodies and limit of detection results were comparable for IL-10 and IL-4 but had varying sensitivity depending on fluorophore for IL-6 as shown below in Tables 5A-5B. IL-6 had the highest level of non-specific background binding of detection antibody which was not found to be fluorophore dependent. However, this high level of non-specific binding led to blank readings with higher coefficients of variation which can lead to larger limits of detection as calculated by 2 times the standard deviation of the blank divided by the slope.

To determine whether the 3-PLEXes functioned comparably to the 1-PLEX assays were run using the same fluorophores, 95% confidence intervals were calculated for each of the three different assays—IL-4, IL-6 and IL-10 as 3-PLEXes and it was determined that the 1-PLEX assays fell within this confidence range. Therefore, a significant amount of sensitivity was not lost when the 1-PLEXes were run as 3-PLEXes (see Tables 5A-5B). Overall, it was demonstrated that the multiplexed analyzer systems can run multiplex assays where single molecules can be counted for three cytokines.

TABLE 5A
Limits of detection for IL-4, IL-6, and
IL-10 using different labeling schemes

| | LoD | | LoD | | LOD |
|---|---|---|---|---|---|
| | pg/ml | | pg/ml | | pg/ml |
| IL-4 Alexa 647 | 0.014 | IL-4 StAv | 0.008 | IL-4 Alexa | 0.008 |
| | | BV421 | | 647 | |
| IL-6 ATTO532 | 0.038 | IL-6 ATTO532 | 0.094 | IL-6 BV421 | 0.139 |
| IL-10 StAv | 0.070 | IL-10 Alexa 647 | 0.049 | IL-10 | 0.093 |
| BV421 | | | | FP532A | |

In Table 5B, three different labeling formats were tested as 3-PLEXES and limits of detection calculated as twice the standard deviation of the blank divided by the slope in the linear region of the concentration curve.

TABLE 5B
Limits of detection for IL-4, IL-6, and
IL-10 using different labeling schemes

| Assay | 3-PLEX LoD 95% Confidence interval | | 1-PLEX LoD |
|---|---|---|---|
| IL-4 Alexa 647 | 0.000-0.014 | pg/ml | 0.005 pg/ml |
| IL-10 StAv | 0.029-0.133 | pg/ml | 0.059 pg/ml |
| BV421 | | | |
| IL-6 ATTO532 | −0.041-0.189 | pg/ml | 0.035 pg/ml |

General Protocol for Immunoassay Test Examples

3-PLEX assays were performed using an immunoassay format. 100 μl/well solutions containing 1 μg/ml capture antibody in 1×PBS were plated into stripwell immunoassay wells and allowed to adsorb overnight at 4° C. The capture antibodies were aspirated and then the plates were washed 3× with 250 μl 1×PBS followed by blocking 3× with 250 μl of SuperBlock.

When used, plasma samples were thawed by warming to room temperature. 300 μl aliquots were placed in an AcroPrep Advance 96 well filter plate and vacuum filtered before usage. Each unique plate run contained standards to generate a standard curve for that plate. 100 μl of either standard or plasma sample was added to the wells and the plates were incubated at 25° C. for two hours on a shaker. All standards were run in triplicate and all plasma samples were run in duplicate. Twelve point standard curves were used that were prepared by serial dilution in standard diluent. The maximum concentrations for IL-4, IL-6, and IL-10 were 10 pg/ml, 100 pg/ml and 400 pg/ml respectively.

After two hours of incubation with standards and samples, the wells were washed four times with wash buffer. 100 μl of detection antibody solution containing 0.1 μg/ml detection antibody in Assay Buffer was then added to the wells. The plates were then incubated at 25° C. for 2 hours on a shaker. After incubation, the wells were washed four times with wash buffer. A final incubation step containing 0.1 μg/ml streptavidin BV421 or detection antibody was performed for 30 minutes at 25° C. on a shaker. The wells were then washed four times with wash buffer before 30 μl of elution buffer was added to each well.

Elution was performed for 10 minutes on a shaker at 25° C. The eluates were then combined with 2 μl of neutralization buffer in a 384 Greiner plate. Bubbles were removed from the wells and the plates were sealed with a foil cover, the reading surface of the plate was wiped clean and the plates were read with the appropriate laser(s) with the 405 nm laser set at 1.2 mW and the 520 and 635 nm lasers at 5 mW.

For 1-PLEX assays, only a single capture antibody at 1 μg/ml, antigen (varying concentrations) and detection antibody at 0.1 μg/ml were used. For 3-PLEX assays, all three capture antibodies were plated at once at 1 μg/ml each, the standard curves contained all three antigens, and all three detection antibodies at 0.1 μg/ml each were added at once with each different assay using a different fluorophore for detection. 1-PLEX assays were run using only the laser needed to excite the fluorophore for that assay. 3-PLEX assays were run using first the 405 nm laser at 1.2 mW, then the 520 nm laser at 5 mW and finally the 635 nm laser at 5 mW.

The concentration of the different analytes in the standard curves was calculated from analysis of DE (detected events), EP (event photons) and TP (total photons) for each well. The concentrations of the unknown samples were interpolated off of the standard curve. The limit of detection for each assay was calculated by dividing two times the standard deviation of the blank sample containing no antigen by the slope of the DE counts for the linear portion of the standard curve to convert the DE counts to a concentration term. The lower limit of quantitation was determined by finding the lowest concentration of the standard curve that had both a % CV below 20% and a recovery between 80-120%.

Sensitivity of Cytokine Assays as Singleplexes

The assay format used for testing of the multiplexed single molecule detection system was a plate immunoassay format. Capture antibodies were allowed to adsorb to multiwell plate surfaces, washed off, blocked, and then samples were loaded onto the plates and allowed to bind. Samples were then washed off and detection antibodies were added. After a final wash step, an elution buffer was added to elute the detection antibodies covalently coupled to fluorophores off the plate and then this solution was neutralized and the number of fluorophores for each different analyte was counted on the multiplexed single molecule detection system.

The model 3-PLEX assay system that was used was IL-4, IL-6 and IL-10. These assays were chosen, in part, because their biologically relevant concentrations span several orders of magnitude and it was desirable that the multiplex could detect a wide range of concentrations without requiring a different sample dilution for each analyte tested. Furthermore, in clinical studies literature where IL-4 and IL-10 have been tested, many times the control samples were below limits of detection. IL-6 has published reference ranges and hence the published results for that range could be compared to the results for the 3-PLEX assay.

Assay conditions were optimized for NaCl concentration, pH, heterophilic blockers, and buffer molarity and antibody pairs used were from kits developed and validated by the Sgx Life Sciences team and Singulex Clinical Laboratory. Capture and detection antibody concentrations were optimized for a plate-based immunoassay and using vendor recommendations. Fluorophores were tested that excited maximally at 405 nm, 520 nm, and 647 nm, namely BV421, ATTO532 and FP532A, and Alexa 647, and that also had minimal spectral overlap for emission. The properties of each fluorophore are outlined in Tables 6A-6B.

TABLE 6A
Assay optimization and assay crosstalk testing

| | Extinction | Quantum | | | |
|---|---|---|---|---|---|
| Fluorophore | Coefficient | Yield | MW | | Photostability |
| BV421 | 2,500,000 | 0.65 | 70 | kD | Low |
| FP532A | 115,000 | 0.90 | ~1,000 | Da | High |
| ATTO532 | 115,000 | 0.90 | ~1,000 | Da | High |
| Alexa 647 | 270,000 | 0.33 | ~1,250 | Da | Medium |

TABLE 6B
Assay optimization and assay crosstalk testing

| | | Limit of detection | % CV of |
|---|---|---|---|
| Assay | Fluorophore | (fg/ml) | background |
| IL-4 | BV421 | 23 | 20% |
| IL-4 | ATTO532 | 18 | 11% |
| IL-4 | Alexa 647 | 15 | 18% |
| IL-10 | BV421 | 43 | 9% |
| IL-10 | FP532A | 7 | 2% |
| IL-10 | Alexa 647 | 29 | 7% |
| IL-6 | BV421 | 39 | 10% |
| IL-6 | ATTO532 | 42 | 12% |
| IL-6 | Alexa 647 | 42 | 7% |

In Table 6A, four fluorophores were tested in the multiplexed single molecule detection system. ATTO532, FP532A, and Alexa 647 were coupled to detection antibodies using NHS ester chemistry. BV421 is much larger and had steric issues if used directly coupled to detection antibodies. Therefore, detection antibodies were labeled with biotin using sulfo-NHS ester chemistry and detection utilized streptavidin coupled to BV421. Table 6B provides a comparison of the limits of detection for 1-PLEX assays run using different fluorophores for detection. The limit of detection was calculated as 2×SD(blank)/slope of a 12-point 2× serial dilution of antigen standard curve.

Impact of Incubation Time on Multiplex Sensitivity

Initial optimization and testing of the 3-PLEX assay utilized two hours of antigen capture time, two hours of detection antibody binding time, and 30 minutes of streptavidin-biotin binding time. One of the advantages of multiplexing is to reduce the amount of time it takes to analyze multiple analytes using a smaller volume of sample than if assays were run separately. Binding avidity and kinetics vary for all antibody/antigen systems and hence it may be beneficial to consider these factors for each system developed. Testing was done to determine the impact of reducing the incubation times for this assay on sensitivity. Reducing the antigen capture time and detection antibody binding time to 30 minutes and leaving the biotin binding time at 30 minutes did not greatly impact the sensitivity of the 3-PLEX assay as indicated in Table 7 below. While there was a slight decrease in the maximum number of single events counted when the assay incubation times were decreased, this did not translate into a large impact on the limits of detection for the analytes. The IL-6 did show the largest impact on the limit of detection with a decrease in assay incubation time but the limit of detection is still well lower than what is found in normal healthy populations as shown in the calculation of reference limits for these assays.

TABLE 7
Reduction of incubation times does not decrease assay sensitivity

| | 2 hr capture/2 hr detection/30 min | | | 30 min capture/30 min detection/30 | | |
|---|---|---|---|---|---|---|
| | biotin binding | | | min biotin binding | | |
| | IL-4 | IL-6 | IL-10 | IL-4 | IL-6 | IL-10 |
| Maximum | 97,524 | 172,865 | 283,852 | 94,031 | 148,870 | 208,860 |
| Single Molecule | | | | | | |
| counts for | | | | | | |
| Highest | | | | | | |
| Standard | | | | | | |
| Limit of | 0.004 | 0.065 | 0.048 | 0.005 | 0.193 | 0.026 |
| detection | | | | | | |
| Slope of | | | | | | |
| Linear Range | 11,674 | 3,042 | 1,804 | 11,529 | 2,379 | 1,246 |
| of Standard | | | | | | |
| Curve | | | | | | |

All conditions of the assays were maintained with the only change being the fluorophore coupled to the detection antibody. Incubation times and temperatures, capture antibody concentrations plated and detection antibody concentrations used were consistent within each specific cytokine assay. There were differences in the limits of detection dependent on the fluorophore used for each assay. This is partially due to the maximal signal intensity that can be obtained from each fluorophores (the extinction coefficients range from 115,000 to 2.5 million and the quantum yields range from 0.3 to 0.9) as well as charge and steric considerations. Overall, all assays have attomolar limits of detection and % CV of the blanks was 20% or less. Therefore, the specific fluorophore used for the 1-PLEX assays does not impact the sensitivity of the assay which is more dependent on assay conditions and antibodies used.

Determination of Reference Limits for IL-4, IL-6 and IL-10 from Healthy Human Plasma Samples in 3-PLEX Format

To effectively use biomarker data, it may be desirable to know what the reference range or limit is for a healthy population of individuals. While many studies use appropriate matched control subpopulations of 20-30 individuals for biomarkers tested, these small populations might not be truly indicative of the reference limit for an analyte. Multiplexed single molecule assays can be used to determine levels of different biomarkers in plasma samples to try and determine previously unknown reference ranges for analytes that have been at or below the limits of detection of previously used assays. The IL-4/IL-6/IL-10 multiplex assay developed to test the new multiplexing single molecule detection instrument was used to determine a reference limit of these three cytokines in a pool of 100 healthy human plasma samples without dilution of any of the plasma samples.

It was determined during prescreening of the human plasma samples that the levels of IL-4 were exceedingly low and close to the limits of quantitation of the IL-4 assay while the IL-6 levels tended to be higher and not as close to the limits of detection. For this reason, the 3-PLEX fluorophore combination chosen was Alexa 647 for IL-4, BV421 for IL-10 and ATTO532 for IL-6 since this combination has the highest level of sensitivity of the different combinations of fluorophores tested.

One of the hallmarks of robust assays is low inter and intraplate variability for samples run. Aliquots of a control plasma sample was run in duplicate on six individual plates over six days using all the same reagents in 3-PLEX format testing for IL-4, IL-6, and IL-10 concentrations. Each run was done on a unique plate with a reference curve generated for all three analytes on each plate. Concentration values were interpolated from the reference curve run on the same plate. The results of interplate and intraplate variability are summarized in FIG. 7.

Plasma samples were run in duplicate over multiple plates and days. Standard curves were also run on each plate and cytokine concentrations were calculated from the standard curve run on the same plate. If values were below the limit of detection they were assigned a value of zero for the reference limit analysis. All values of IL-6 and IL-10 could be quantified. Nine plasma samples out of 100 had non-detectable levels of IL-4. The 95% reference limits, number of samples used to generate the values after a Tukey test to remove far outliers, and the 90% confidence interval of the reference limit generated by quantile testing for non-parametric data using Analyse It version 4.10 are listed for the three cytokines tested in Tables 8A-8B.

TABLE 8A

| | Average Intraplate Variability | Interplate Variability |
|---|---|---|
| IL-4 | 11.1% | 9.3% |
| IL-6 | 3.2% | 1.4% |
| IL-10 | 10.7% | 6.0% |

TABLE 8B

| | Samples used in | 95% Reference limit | |
|---|---|---|---|
| Cytokine | calculation | (pg/ml) | 90% CI (pg/ml) |
| IL-4 | 80 | 0.61 | 0.47-0.65 |
| IL-6 | 93 | 6.53 | 5.09-7.19 |
| IL-10 | 95 | 1.08 | 0.84-1.49 |

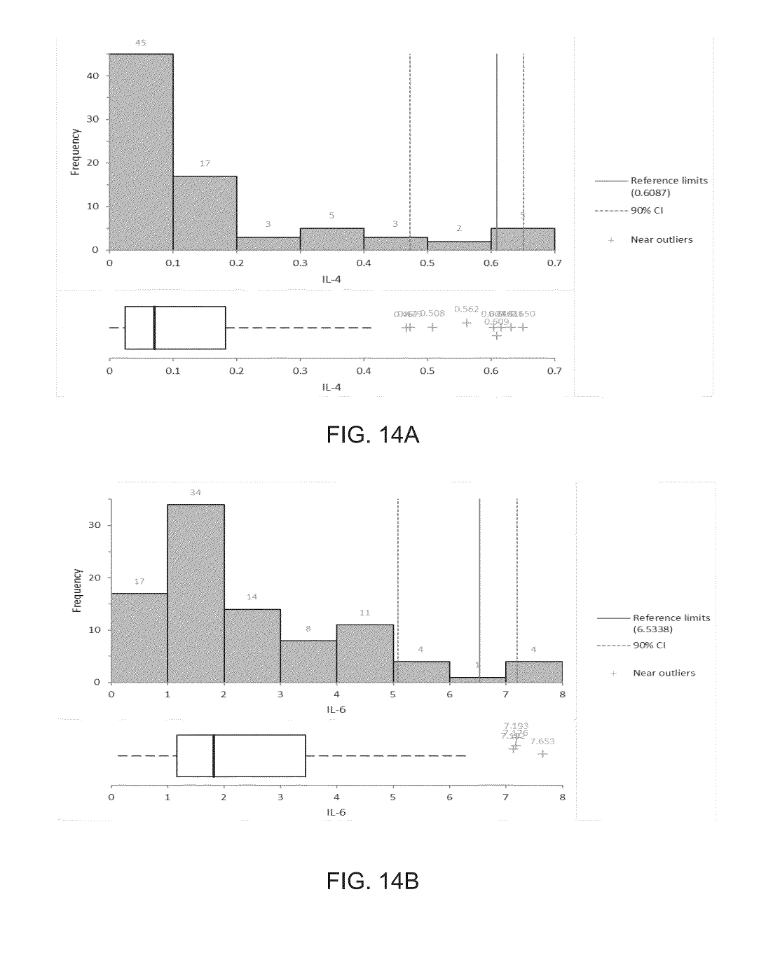

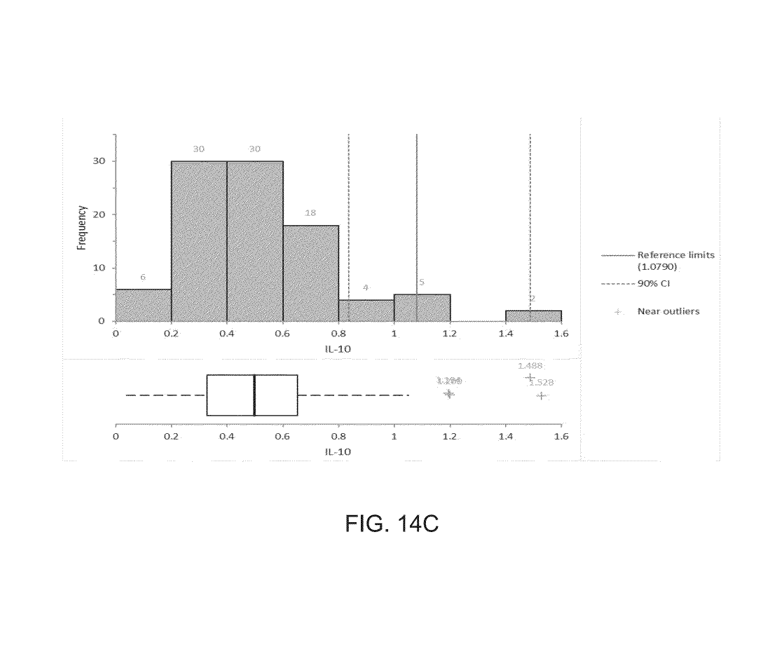

FIGS. 14A-14C show graphical representations of reference limit calculations for 100 self-declared healthy human plasma samples tested for IL-4, IL-6 and IL-10. In particular, FIG. 14A shows a summary of interplate and intraplate variability, FIG. 14B shows a summary of the results for calculation of reference limits, and FIG. 14C shows histograms for IL-4, IL-6 and IL-10 reference limit calculations.

The multiplex single molecule detection system was capable of detecting and quantifying 100% of the plasma samples tested for IL-10 with a 95% reference limit of 1.08 pg/ml. Diagnostic companies such as Viracor-IBT report that the reference limit for IL-10 in plasma is <2 pg/ml and tests this cytokine using the Meso Scale Discovery Sector Imager 2400 (http://www.viracoribt.com/Test-Catalog/Detail/Interleukin-10-IL-10-Plasma-1222)). Two pg/ml is the minimum limit of detection of the assay and hence the healthy normal population all fall below the detectable limit for this assay. Other diagnostic labs such as Quest use an immunoassay format and have a reference range <4.6 pg/ml (http://www.questdiagnostics.com/testcenter/BUOrderInfo.action?tc=27160&labCode=QDV (www.questdiagnostics.com)). Literature values for normal healthy individuals varies considerably for IL-10 when measured by ELISA ranging from approximately 5 pg/ml to 1.6 pg/ml when measured using a different ELISA assay.

The multiplex single molecule assay reference limit for IL-4 was 0.61 pg/ml determined with only nine of 100 samples below the limit of detection. From literature from the diagnostic company Viracor-IBT (http://www.viracoribt.com/Test-Catalog/Detail/Interleukin-4-IL-4-Plasma-1227) using the Meso Scale Discovery Sector Imager 2400 for IL-4 detection results in a reference limit less than 0.4 pg/ml, below the limit of detection for this assay. The IL-4 reference limit calculation resulted in the removal of twenty samples from the calculation after Tukey outlier analysis was deployed. While this seemed like a very large number of far outliers, this can potentially be explained by the large range of IL-4 values found in the literature depending on the ethnic background of the individual tested ranging from close to zero for Western European populations to 5 pg/ml and greater for Asian populations. Because of HIPAA regulations, the ethnicity of the individual donors was not known for the 100 samples tested to generate the 95% reference limit for IL-4 other than the samples were from a self-declared healthy population from Florida. The IL-4 values for the 20 samples that were removed after Tukey analysis included four samples between 9-10.5 pg/ml, six samples between 3-4.5 pg/ml, eight samples between 1.5-2 pg/ml and two samples that were 0.9 pg/ml. These could have been due to ethnic differences or other population effects currently not known, not truly being healthy, or some other cause not recorded or used as an elimination factor in the screening of individuals. Having a robust methodology such as the single molecule detection with multiplexing to accurately and reproducibly be able to measure IL-4 in a range that has not been capable of accurate detection in the past could allow some of these population analyses to be performed. With respect to IL-6, the 3-PLEX assay produced clinically valid values.

Although preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

What is claimed is:

1. An analyzer, comprising:
(a) an electromagnetic radiation source for providing electromagnetic radiation to a sample container for a sample, wherein the electromagnetic radiation source provides electromagnetic radiation at wavelengths within at least an excitation band of a first label corresponding to a first target molecule and within an excitation band of a second label corresponding to a second target molecule;
(b) a system for directing the electromagnetic radiation from the electromagnetic radiation source to an interrogation space in the sample;
(c) a translating system for translating the interrogation space through at least a portion of the sample, thereby forming a moveable interrogation space; and
(d) a detector system comprising a first detector for detecting electromagnetic radiation emitted in the interrogation space by the first label if the first label is present in the interrogation space and a second detector for detecting electromagnetic radiation emitted in the interrogation space by the second label if the second label is present in the interrogation space.

2. The analyzer of claim 1, wherein a single electromagnetic radiation source provides electromagnetic radiation at wavelengths that are within the excitation bands of at least the first label and the second label.

3. The analyzer of claim 1, wherein the electromagnetic radiation source comprises a first source that excites the first label and a second source that excites the second label.

4. The analyzer of claim 1, wherein the excitation bands of the first label and the second a label do not overlap.

5. The analyzer of claim 1, wherein the excitation band of the first label overlaps the excitation band of the second label

6. The analyzer of claim 1, further comprising a processor configured to:
determine a first threshold photon value for the first label corresponding to a background signal in the interrogation space at an emission wavelength of the first label,
determine a second threshold photon value for the second label corresponding to a background signal in the interrogation space at an emission wavelength of the second label,
receive a first photon count signal from the first detector comprising a photon count value for the first label detected in the interrogation space in each bin of a first plurality of bins,
receive a second photon count signal from the second detector comprising a photon count value for the second label detected in the interrogation space in each bin of a second plurality of bins,
determine the first target molecule by determining the first label in the interrogation space by identifying each bin of the first plurality of bins having a photon value for the first label greater than the first threshold value; and
determine the second target molecule by determining the second label in the interrogation space by identifying each bin of the second plurality of bins having a photon value for the second label greater than the second threshold value.

7. The analyzer of claim 6, wherein the processor is further configured to:
determine a concentration of the first target molecule as a function of a sum of the number of bins having a photon value for the first label that is greater than the threshold value; and
determine a concentration of the second target molecule as a function of a sum of the number of bins having a photon value for the second label that is greater than the threshold value.

8. The analyzer of claim 6, wherein the first plurality of bins and the second plurality of bins comprise the same bins.

9. The analyzer of claim 6, wherein the first plurality of bins is different than the second plurality of bins.

10. The analyzer of claim 1, wherein the system for directing the electromagnetic radiation from the electromagnetic radiation source to the interrogation space comprises:
a first filter for directing electromagnetic radiation at a first wavelength to the interrogation space, wherein the first wavelength is within the excitation band of the first label; and
a second filter for directing electromagnetic radiation at a second wavelength to the interrogation space, wherein the second wavelength is within the excitation band of the second label.

11. An analyzer, comprising:
(a) an electromagnetic radiation source for providing electromagnetic radiation to a sample container for a sample, wherein the electromagnetic radiation source provides electromagnetic radiation at wavelengths within at least an excitation band of a first fluorescent moiety and within an excitation band of a second fluorescent moiety;
(b) a system for directing the electromagnetic radiation from the electromagnetic radiation source to an interrogation space in the sample;
(c) a translating system for translating the interrogation space through at least a portion of the sample, thereby forming a moveable interrogation space;
(d) a detector system comprising a first detector for detecting electromagnetic radiation emitted in the interrogation space by the first fluorescent moiety, a second detector for detecting electromagnetic radiation emitted in the interrogation space by the second fluorescent moiety during a plurality of bin times,
(e) a processor configured to:
determine a first threshold photon value corresponding to a background signal in the interrogation space at an emission wavelength of the first label,
determine a second threshold photon value corresponding to a background signal in the interrogation space at an emission wavelength of the second label,
determine a first analyte by determining the first fluorescent moiety corresponding to the first analyte in the interrogation space by identifying each bin of a first plurality of bins having a photon value for the first moiety greater than the first threshold value;
determine a second analyte by determining the second fluorescent moiety corresponding to the second analyte in the interrogation space by identifying each bin of a second plurality of bins having a photon value for the second moiety greater than the second threshold value; and
determine a third analyte by determining a combination of the first fluorescent moiety and the second fluorescent moiety corresponding to the third analyte in the interrogation space by identifying each bin of a third plurality of bins having a photon value for each of the first moiety and the second moiety greater than the first and the second threshold values.

12. The analyzer of claim 11, wherein the processor is further configured to:
determine a concentration of the first analyte as a function of a sum of the number of bins having a photon value for the first moiety that is greater than the threshold value;
determine a concentration of the second analyte as a function of a sum of the number of bins having a photon value for the second moiety that is greater than the threshold value, and
determine a concentration of the third analyte as a function of a sum of the number of bins having a photon values for both of the first moiety and the second moiety that are greater than the first and the second threshold values.

13. An analyzer, comprising:
(a) a first electromagnetic radiation source for providing electromagnetic radiation at an excitation wavelength to a sample container for a sample and a second electromagnetic radiation source for providing electromagnetic radiation at an excitation wavelength to the sample container;
(b) a system for directing the electromagnetic radiation from the first electromagnetic radiation source and the second electromagnetic radiation source to at least one interrogation space in the sample;
(c) a translating system for translating the interrogation space through at least a portion of the sample, thereby forming a moveable interrogation space; and
(d) a detection system for detecting electromagnetic radiation, wherein the detection system is configured to detect electromagnetic radiation from the interrogation space emitted by:
(i) a first label corresponding to a single molecule of the first target molecule if the first label is present in the at least one interrogation space, wherein the first label has an excitation wavelength overlapping the excitation wavelength of the first electromagnetic radiation source; and
(ii) the second label corresponding to a single molecule of the second target molecule if the second label is present in the at least one interrogation space, wherein the second label has an excitation wavelength overlapping the excitation wavelength of the second electromagnetic radiation source.

14. The analyzer of claim 13, wherein the excitation wavelength of the first electromagnetic radiation source does not overlap with the excitation wavelength of the second label, and wherein the excitation wavelength of the second electromagnetic radiation source does not overlap with the excitation wavelength of the first label.

15. The analyzer of claim 13, further comprising a processor configured to:
determine a first threshold photon value corresponding to a background signal in the at least one interrogation space at an emission wavelength of the first label,
determine a second threshold photon value corresponding to a background signal in the at least one interrogation space at an emission wavelength of the second label,
determine the first target molecule by determining the presence of the first label corresponding to the first target molecule in the at least one interrogation space in each bin of a first plurality of bins by identifying bins having a photon value for the first label greater than the first threshold value when the first electromagnetic radiation source provides electromagnetic radiation to the at least one interrogation space; and
determine the second target molecule by determining the presence of the second label corresponding to the second target molecule in the at least one interrogation space in each bin of a second plurality of bin times by identifying bins having a photon value for the second label greater than the threshold value when the second electromagnetic radiation source provides electromagnetic radiation to the at least one interrogation space.

16. The analyzer of claim 15, wherein each bin of the first plurality of bins is different than each bin of the second plurality of bins.

17. The analyzer of claim 15, further comprising a third electromagnetic radiation source for providing electromagnetic radiation at a third excitation wavelength to at least one interrogation space, and wherein the detection system is further configured to detection electromagnetic radiation emitted by a third label corresponding to a third target molecule.

18. The analyzer of claim 17, wherein the processor is further configured to
determine a third threshold photon value corresponding to a background signal in the at least one interrogation space at an emission wavelength of the third label, and
determine the third target molecule by determining the presence of the third label corresponding to the third target molecule in the at least one interrogation space in each bin of a third plurality of bin times by identifying bins having a photon value for the third label greater than the third threshold value when the third electromagnetic radiation source provides electromagnetic radiation to the at least one interrogation space.

19. The analyzer of claim 18, wherein each bin of the first, second and third plurality of bins are different bins.

20. The analyzer of claim 13, wherein the detection system comprises a first detector configured to detect electromagnetic radiation emitted by the first label and a second detector configured to detect electromagnetic radiation emitted by the second label.

21. The analyzer of claim 20, wherein the detection system comprises a first detector configured to detect electromagnetic radiation emitted by the first label, and a second detector configured to detect electromagnetic radiation emitted by the second label, and a third detector configured to detect electromagnetic radiation emitted by the third label.

22. The analyzer of claim 20, wherein the system for directing electromagnetic radiation to the at least one interrogation space in the sample is configured to direct electromagnetic radiation from the first electromagnetic radiation source to a first interrogation space and to direct electromagnetic radiation from the second electromagnetic radiation source to a second interrogation space, wherein the first detector is configured to detect electromagnetic radiation from the first interrogation space and the second detector is configured to detect electromagnetic radiation from the second interrogation space.

23. A method for determining multiple target molecules, the method comprising:
(a) directing electromagnetic radiation from an electromagnetic radiation source to an interrogation space in a sample at a first wavelength within at least an excitation band of a first label corresponding to a first target molecule and a second wavelength within at least an excitation band of a second label corresponding to a second target molecule;
(b) detecting the first label in the interrogation space located at a first position in the sample;
(c) detecting the second label in the interrogation space at the first position;
(d) translating the interrogation space through the sample to a subsequent position in the sample;
(e) detecting the first label in the interrogation space located at the subsequent position in the sample;
(f) detecting second label in the interrogation space located at the subsequent position in the sample; and
(g) repeating steps (d), (e) and (f) as required to detect the first label and the second label in more than one position of the sample, thereby determining the first target molecule and the second target molecule.

24. The method of claim 23, further comprising:
determining a first threshold photon value for the first label corresponding to a background signal in the interrogation space at an emission wavelength of the first label in at least one position in the sample,
determining a second threshold photon value for the second label corresponding to a background signal in the interrogation space at an emission wavelength of the second label in at least one position in the sample,
determining a first photon count signal from the first detector comprising a photon count value for the first label detected in the interrogation space in each bin of a first plurality of bins corresponding to the more than one position in the sample,
determining a second photon count signal from the second detector comprising a photon count value for the second label detected in the interrogation space in each bin of a second plurality of bins corresponding to the more than one position in the sample,
determining the first target molecule by determining the first label in the interrogation space by identifying each bin of the first plurality of bins having a photon value for the first label greater than the first threshold value; and
determining the second target molecule by determining the second label in the interrogation space by identifying each bin of the second plurality of bins having a photon value for the second label greater than the second threshold value.

25. The method of claim 23, wherein the first label is detected when electromagnetic radiation at the first wavelength is directed to the interrogation space and wherein the second label is detected when electromagnetic radiation at the second wavelength is directed to the interrogation space, wherein electromagnetic radiation at the first wavelength and electromagnetic radiation at the second wavelength are directed to the interrogation space in series.

26. The method of claim 25, wherein the first label and the second label are detected by a single detector.

27. The method of claim 25, wherein the first plurality of bins is different than the second plurality of bins.

28. The method of claim 23, wherein the electromagnetic radiation source comprises a first source that provides electromagnetic radiation at the first wavelength and second source that provides electromagnetic radiation at the second wavelength.

29. The method of claim 23 wherein the detecting the first label and the second label comprises detecting the first label with a first detector and detecting the second label with a second detector, wherein the first detector does not detect an emission wavelength of the second label and the second detector does not detect an emission wavelength of the first label.

30. A method for determining a target molecule, comprising:
(a) directing electromagnetic radiation from a first electromagnetic radiation source at a first wavelength to a first interrogation space in a sample and directing electromagnetic radiation from a second electromagnetic radiation source at a second wavelength to a second interrogation space in the sample, wherein the first and second interrogation spaces are within a focus of a single objective of a detector;
(b) detecting with a first detector the first label corresponding to a first target molecule in the first interrogation space at the first position in the sample; wherein the first label has an excitation wavelength within the first wavelength, wherein electromagnetic radiation emitted in the first interrogation space is directed to the first detector;
(c) detecting with a second detector a second label corresponding to second target molecule in the second interrogation space at the first position in the sample, wherein the second label has an excitation wavelength within the second wavelength, wherein electromagnetic radiation emitted in the second interrogation space is directed to the second detector;
(d) translating the interrogation space through the sample to a subsequent position in the sample;
(e) detecting with the first detector the first label in the first interrogation space at the subsequent position in the sample;
(f) detecting with the second detector the second label in the second interrogation space at the subsequence position in the sample; and
(g) repeating steps (d), (e) and (f) as required to determine the first target molecule and the second target molecule.

31. The method of claim 30, further comprising:
determining a first threshold photon value for the first label corresponding to a background signal in the interrogation space at an emission wavelength of the first label in at least one position in the sample,
determining a second threshold photon value for the second label corresponding to a background signal in the interrogation space at an emission wavelength of the second label in at least one position in the sample,
determining a first photon count signal from the first detector comprising a photon count value for the first label detected in the interrogation space in each bin of a first plurality of bins corresponding to the more than one position in the sample,
determining a second photon count signal from the second detector comprising a photon count value for the second label detected in the interrogation space in each bin of a second plurality of bins corresponding to the more than one position in the sample,
determining the first target molecule by determining the first label in the interrogation space by identifying each bin of the first plurality of bins having a photon value for the first label greater than the first threshold value; and
determining the second target molecule by determining the second label in the interrogation space by identifying each bin of the second plurality of bins having a photon value for the second label greater than the second threshold value.
